(19) United States
(12) Patent Application Publication
(10) Pub. No.: US 2025/0236680 A1
(43) Pub. Date: Jul. 24, 2025

(54) ANTI-PSMA ANTIBODIES, VARIANTS, AND USES THEREOF

(71) Applicant: XI AN ORIMAB BIOTECHNOLOGY COMPANY LTD., Xi An, ShaanXi Province (CN)

(72) Inventors: Aizhi Zhao, Wallingford, PA (US); Weihong Wen, Xi'an (CN)

(73) Assignee: Xi An OriMAb Biotechnology Company Ltd., Xi An, ShaanXi (CN)

(21) Appl. No.: 18/852,661

(22) PCT Filed: Mar. 28, 2023
(86) PCT No.: PCT/US2023/065017

§ 371 Date: Sep. 30, 2024

Related U.S. Application Data
(60) Provisional application No. 63/325,330, filed on Mar. 30, 2022.

(51) Int. Cl. C07K 16/30; A61K 39/00; A61K 49/00; A61K 49/16; A61K 51/10; A61P 35/00; C07K 14/725; G01N 33/574

(52) U.S. Cl. CPC C07K 16/3069; A61K 49/0058; A61K 49/16; A61K 51/1072; A61P 35/00; C07K 14/7051; G01N 33/57484; A61K 2039/505; A61K 2123/00; C07K 2317/31; C07K 2317/41; C07K 2317/732; C07K 2317/77; C07K 2317/92; C07K 2319/02; C07K 2319/03

(57) ABSTRACT

PSMA is a prostate cancer marker, and cancers with higher malignancy or metastasis, or resistant to androgen deprivation therapy, usually express much higher PSMA. The present invention relates to the improved anti-PSMA antibodies or antigen-binding fragments thereof, related nucleic acids, vectors, cells, compositions, kits, and the methods of use thereof.

### CROSS REFERENCE TO RELATED APPLICATION

This application claims priority to U.S. Provisional Application No. 63/325,330 filed on Mar. 30, 2022. The content of the application is incorporated herein by reference in its entirety.

### REFERENCE TO A SEQUENCE LISTING

This application incorporates by reference the Sequence Listing submitted in Computer Readable Form as file SeqList_168276.00301, created on Mar. 27, 2023 and containing 81,222 bytes.

### FIELD OF THE INVENTION

This invention relates to improved anti-PSMA antibodies and methods of use thereof.

### BACKGROUND OF THE INVENTION

Prostate specific membrane antigen (PSMA) has been confirmed as a specific marker for prostate epithelial cells, which laid the foundation for development of prostate cancer-targeted precision medicine. Histological studies indicated that almost all prostate cancers express PSMA, and cancers with higher malignancy or metastasis, or resistant to androgen deprivation therapy, usually express much higher PSMA (Ananias, H. J., et al. (2009) Prostate, 69(10): 1101-8; Wright, G. L., Jr., et al. (1995) Urol Oncol, 1(1): 18-28; Wright, G. L., Jr., et al. (1996) Urology, 48(2): 326-34; Sweat, S. D., et al. (1998) Urology, 52(4): 637-40). It is of note that the PSMA expression levels in normal tissues are 100-1000 times lower than in tumor (Sokoloff, R. L., et al. (2000) Prostate, 43(2): 150-7), and these normal tissues are usually not easily accessible to circulating antibodies (Troyer, J. K. et al. (1995) Int J Cancer, 62(5): 552-8), which further assured the safety of PSMA targeted imaging and therapy. In addition to prostate, PSMA is also highly expressed in neovasculature in many solid tumors while is absent in normal vessels (Sokoloff, R. L., et al. (2000) Prostate, 43(2): 150-7). Therefore, PSMA is an ideal marker not only for prostate cancer, but also for neovasculature targeted therapy for other solid tumors.

Antibodies are the most efficient tool for tumor targeting in that they can specifically recognize tumor-related or tumor-specific antigens expressed on tumor cells, which opens avenues for antibody-based precision medicine, including tumor-targeted imaging and therapy, such as optical, PET, SPECT, or MRI imaging for early tumor detection, antibody-drug conjugate, and radiotherapy, bi-specific or multi-specific antibodies, chimeric antigen receptor T cell or NK cell therapies for cancer treatment.

However, the stability of protein drugs such as antibodies is adversely affected by many different factors. Degradation of proteins and subsequent reduction in protein activity is a recurring problem in the pharmaceutical industry. Accordingly, there remains a need for antibodies as therapeutic agents with improved stability.

### SUMMARY OF THE INVENTION

This disclosure address the above need in a number of aspects. In one aspect, this disclosure provides an antibody or antigen-binding fragment thereof or a variant thereof that specifically binds to PSMA, such as human PSMA. In some embodiments, the antibody or antigen-binding fragment thereof comprises: (i) a light chain variable region (LCVR) comprising three light chain complementarity determining regions (LCDRs) (LCDR1, LCDR2, and LCDR3), wherein the LCDR1 comprises the amino acid sequence of SEQ ID NO: 9, the LCDR2 comprises the amino acid sequence of SEQ ID NO: 13 or 41, and the LCDR3 comprises the amino acid sequence of SEQ ID NO: 17; and/or (ii) heavy chain variable region (HCVR) three heavy chain complementarity determining regions (HCDRs) (HCDR1, HCDR2, and HCDR3), comprising wherein the HCDR1 comprises the amino acid sequence of SEQ ID NO: 25 or 45, the HCDR2 comprises the amino acid sequence of SEQ ID NO: 29, and the HCDR3 comprises the amino acid sequence of SEQ ID NO: 33 or 49, wherein the variant comprises one or more conservative amino acid substitutions in the LCDR1, the LCDR2, the LCDR3, the HCDR1, the HCDR2, or the HCDR3.

In some embodiments, the LCDR1 comprises the amino acid sequence of SEQ ID NO: 9, the LCDR2 comprises the amino acid sequence of SEQ ID NO: 13, the LCDR3 comprises the amino acid sequence of SEQ ID NO: 17; the HCDR1 comprises the amino acid sequence of SEQ ID NO: 25, the HCDR2 comprises the amino acid sequence of SEQ ID NO: 29, and the HCDR3 comprises the amino acid sequence of SEQ ID NO: 33.

In some embodiments, the LCDR1 comprises the amino acid sequence of SEQ ID NO: 9, the LCDR2 comprises the amino acid sequence of SEQ ID NO: 13, the LCDR3 comprises the amino acid sequence of SEQ ID NO: 17; the HCDR1 comprises the amino acid sequence of SEQ ID NO: 45, the HCDR2 comprises the amino acid sequence of SEQ ID NO: 29, and the HCDR3 comprises the amino acid sequence of SEQ ID NO: 33.

In some embodiments, the LCDR1 comprises the amino acid sequence of SEQ ID NO: 9, the LCDR2 comprises the amino acid sequence of SEQ ID NO: 41, the LCDR3 comprises the amino acid sequence of SEQ ID NO: 17; the HCDR1 comprises the amino acid sequence of SEQ ID NO: 45, the HCDR2 comprises the amino acid sequence of SEQ ID NO: 29, and the HCDR3 comprises the amino acid sequence of SEQ ID NO: 33.

In some embodiments, the LCDR1 comprises the amino acid sequence of SEQ ID NO: 9, the LCDR2 comprises the amino acid sequence of SEQ ID NO: 13, the LCDR3 comprises the amino acid sequence of SEQ ID NO: 17; the HCDR1 comprises the amino acid sequence of SEQ ID NO: 45, the HCDR2 comprises the amino acid sequence of SEQ ID NO: 29, and the HCDR3 comprises the amino acid sequence of SEQ ID NO: 49.

In some embodiments, the light chain variable region comprises one or more of the amino acid sequences of SEQ ID NOs: 5, 7, 11, 15, 19, 39, 43, 61, 63, 65, 67, and 69.

In some embodiments, the heavy chain variable region comprises one or more of the amino acid sequences of SEQ ID NOs: 21, 23, 27, 31, 35, 47, 51, 53, 55, 57, 59, and 68.

In some embodiments, the light chain variable region comprises an amino acid sequence with at least 75% identity to the amino acid sequence of SEQ ID NO: 5, or comprises the amino acid sequence of SEQ ID NO: 5; and/or the heavy chain variable region comprises an amino acid sequence with at least 75% identity to the amino acid sequence of SEQ ID NO: 21, or comprises the amino acid sequence of SEQ ID NO: 21.

In some embodiments, the light chain variable region comprises an amino acid sequence with at least 75% identity to the amino acid sequence of SEQ ID NO: 70, or comprises the amino acid sequence of SEQ ID NO: 70; and/or the heavy chain variable region comprises an amino acid sequence with at least 75% identity to the amino acid sequence of SEQ ID NO: 72, or comprises the amino acid sequence of SEQ ID NO: 72.

In some embodiments, the light chain variable region comprises an amino acid sequence with at least 75% identity to the amino acid sequence of SEQ ID NO: 71, or comprises the amino acid sequence of SEQ ID NO: 71; and/or the heavy chain variable region comprises an amino acid sequence with at least 75% identity to the amino acid sequence of SEQ ID NO: 73, or comprises the amino acid sequence of SEQ ID NO: 73.

In some embodiments, the light chain variable region comprises an amino acid sequence with at least 75% identity to the amino acid sequence of SEQ ID NO: 5, or comprises the amino acid sequence of SEQ ID NO: 5; and/or the heavy chain variable region comprises an amino acid sequence with at least 75% identity to the amino acid sequence of SEQ ID NO: 74, or comprises the amino acid sequence of SEQ ID NO: 74.

In some embodiments, the light chain variable region and the heavy chain variable region comprise a LCVR and HCVR amino acid sequence pair of SEQ ID NOs: 5 and 21, SEQ ID NOs: 70 and 72, SEQ ID NOs: 71 and 73, or SEQ ID NOs: 5 and 74.

In some embodiments, the antibody or antigen-binding fragment thereof or variant thereof comprises a heavy chain/light chain sequence pair of SEQ ID NOs: 75 and 77, SEQ ID NOs: 76 and 78, SEQ ID NOs: 69 and 68, or SEQ ID NOs: 75 and 79.

In some embodiments, the antibody or antigen-binding fragment thereof or variant thereof comprises at least one of the amino acid sequences of SEQ ID NOs: 3 and 37.

In some embodiments, the antibody or antigen-binding fragment thereof is selected from IgG1, IgG2, IgG3, IgG4, IgM, IgA1, IgA2, IgAsec, IgD, and IgE; or has a wild type or afucosylated immunoglobulin constant and/or variable domain of IgG1, IgG2, IgG3, IgG4, IgM, IgA1, IgA2, IgAsec, IgA or IgE.

In some embodiments, the antibody or antigen-binding fragment thereof is a recombinant antibody, a monoclonal antibody, a polyclonal antibody, a mixture of monoclonal and/or polyclonal antibodies, a human antibody, a humanized antibody, or a chimeric antibody.

In some embodiments, the antibody or antigen-binding fragment thereof or variant thereof is conjugated to a cytotoxic agent, a drug, a growth inhibitory agent, a toxin, an enzyme, a tag, a label, a radioactive isotope or a nanoparticle.

In another aspect, this disclosure also provides a bispecific or multiple-specific antibody or antigen-binding fragment thereof that binds two or more different epitopes on the same or different antigens. In some embodiments, one of the epitopes is on human prostate specific membrane antigen (PSMA), and the bispecific or multiple-specific antibody comprises the light chain variable region and/or the heavy chain variable region of the antibody or antigen-binding fragment thereof or variant thereof, as described herein.

In another aspect, this disclosure further provides an isolated chimeric antigen receptor (CAR). In some embodiments, the CAR comprises the antibody or antigen-binding fragment thereof or variant thereof described herein, a transmembrane domain, and an intracellular signaling domain comprising one or more stimulatory domains.

Also within the scope of this disclosure are (a) a nucleic acid molecule encoding a polypeptide chain of the antibody or antigen-binding fragment thereof or variant thereof, the bispecific or multiple-specific antibody or antigen-binding fragment thereof, or the CAR of claim, as described herein; (b) a vector comprising the nucleic acid molecule described herein; (c) a cell comprising the nucleic acid molecule or the vector, as described herein.

In another aspect, this disclosure provides a method of preparing an antibody or antigen-binding fragment thereof. In some embodiments, the method comprises (i) obtaining the cell described herein; (ii) culturing the cell in a medium under conditions permitting expression of a polypeptide encoded by the nucleic acid molecule and assembling of an antibody or fragment thereof; and (iii) purifying the antibody or fragment from the cultured cell or the medium of the cell.

In another aspect, this disclosure additionally provides a composition comprising the antibody or antigen-binding fragment thereof or variant thereof of, the bispecific or multiple-specific antibody or antigen-binding fragment thereof, the CAR, the nucleic acid molecule, the vector, or the cell, as described herein, and optionally a pharmaceutically acceptable carrier.

In another aspect, this disclosure additionally provides a kit comprising the antibody or antigen-binding fragment thereof or variant thereof, the bispecific or multiple-specific antibody or antigen-binding fragment thereof, the CAR, the nucleic acid molecule, the vector, the cell, or the composition, as described herein.

In another aspect, this disclosure also provides use of the composition described herein for the manufacture of a medicament for diagnosis, prophylaxis, treatment, or combination thereof of a condition resulting from a proliferative disease associated with expression of PSMA in a subject.

In another aspect, this disclosure further provides a method of preventing or treating a proliferative disease associated with expression of PSMA in a subject. In some embodiments, the method comprises administering to the subject an effective amount of the antibody or antigen-binding fragment thereof or variant thereof, the bispecific or multiple-specific antibody or antigen-binding fragment thereof, the CAR, the nucleic acid molecule, the vector, the cell, or the composition, as described herein.

In some embodiments, the method further comprises administering to the subject a second agent or therapy. In some embodiments, the second agent or therapy comprises an anti-cancer agent. In some embodiments, the second agent or therapy is administered to the subject before, after, or concurrently with the antibody or antigen-binding fragment thereof or variant thereof, the bispecific or multiple-specific antibody or antigen-binding fragment thereof, the CAR, the nucleic acid molecule, the vector, the cell, or the composition, as described herein.

In another aspect, this disclosure also provides a method of identifying a subject having a disease or condition associated with the expression of PSMA. In some embodiments, the method comprises: (a) providing a tissue sample from the subject; (b) contacting the sample with the antibody or antigen-binding fragment thereof or variant thereof; and (c) determining binding of the antibody or antigen-binding fragment thereof or variant thereof to the tissue sample, wherein the binding of the antibody or antigen-binding fragment thereof or variant thereof to the tissue sample is indicative of the presence of a disease condition associated with the expression of PSMA in the subject.

In yet another aspect, this disclosure also provides a method of imaging that targets PSMA. The method comprises administering to a subject in need thereof the antibody or antigen-binding fragment thereof or variant thereof or the composition, wherein the antibody or antigen-binding fragment thereof or variant is linked to a reagent. In some embodiments, the reagent comprises a photoactivatable agent, a fluorophore, a radioisotope, a bioluminescent protein, a bioluminescent peptide, a fluorescent tag, a fluorescent protein, a fluorescent peptide, an imaging contrast, an enzyme, a nuclear magnetic resonance active reagent, or a nanoparticle.

In some embodiments, the disease comprises a cancer. In some embodiments, the cancer comprises a prostate cancer.

In some embodiments, the subject is a mammal, e.g., a human.

The foregoing summary is not intended to define every aspect of the disclosure, and additional aspects are described in other sections, such as the following detailed description.

The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts the results of experiments measuring affinity for PSMAb and PSMAbLm by Capture ELISA. Data showed that the affinity of PSMAbLm is about 2.3-fold higher than that of PSMAb (Kd=0.0479 nM (PSMAbLm) vs. Kd=0.1088 nM (PSMAb)).

FIG. 2 depicts the ADCC activity of PSMAb-WT, PSMAb-AF, and PSMAbLm-AF on target cells. ADCC was investigated using a CD16-Jurkat reporter cells with PSMA+CHO cell as a target. Abbreviations: PSMAb-WT: wild type PSMAb with normal glycosylation; PSMAb-AF: afucosylated PSMAb; PSMAbLm-AF: afucosylated PSMAb with N=>S mutation in light chain CDR3 region.

FIGS. 3A and 3B depict the binding of PSMAb and PSMAbLm on LnCap cells.

FIG. 3A shows the cell populations and FIG. 3B shows the fluorescence mean intensity of each antibody concentration.

FIGS. 4A, 4B, 4C, and 4D depict the internalization of PSMAb and PSMAbLm into LnCap cells. FIGS. 4A to 4C show the cell population with internalized antibodies at the antibody concentrations of 0.001-0.009 nM, 0.027-0.247 nM, and 0.74-6.67 nM respectively. FIG. 4D shows the statistic fluorescence mean intensity of the cells with internalized antibody at different concentrations.

### DETAILED DESCRIPTION

This disclosure relates to compositions and methods for treating and/or preventing cancer. It is based in part upon the discovery of optimized antibodies or antigen-binding fragments thereof that specifically bind to an epitope in the extracellular domain of PSMA (e.g., human PSMA).

In one aspect, this disclosure provides g an antibody or antigen-binding fragment thereof that binds to PSMA. The disclosed anti-PSMA antibodies or antigen-binding fragments thereof have superior stability and effectiveness compared to the parent antibody. Also within the scope of this disclosure are a bispecific antibody, an antibody-drug conjugate, a PSMA-targeting imaging agent, a chimeric antigen receptor, a cell expressing a chimeric antigen receptor, and the like.

In some embodiments, this disclosure provides a composition comprising an antibody or antigen-binding fragment thereof that specifically binds to the extracellular domain of PSMA. The antibody or antigen-binding fragment thereof can be formulated as therapeutic and diagnostic compositions that target PSMA present in prostate cancer and/or in the neovasculature of other solid tumors. In one embodiment, the composition may include an antibody-drug conjugate (ADC), wherein the antibody or antibody or antigen-binding fragment thereof targets the drug to the tumor location. In one embodiment, the composition comprises a bispecific antibody. In some embodiments, the bispecific antibody comprises a first antigen-binding arm that specifically binds to PSMA and a second antigen-binding arm that binds to a T-cell antigen (e.g., CD3). In one embodiment, the composition is a chimeric antigen receptor that comprises the antibody or antigen-binding fragment thereof that specifically binds to PSMA.

In another aspect, this disclosure also provides an isolated nucleic acid encoding an antibody or antigen-binding fragment thereof, bispecific antibody, or chimeric antigen receptor, as disclosed herein. Also within the scope of this disclosure are a cell that is modified to express an antibody or antigen-binding fragment thereof, bispecific antibody, or chimeric antigen receptor, as disclosed herein, and a cell culture comprising the cell.

This disclosure additionally provides a method for treating or preventing cancer, including but not limited to prostate cancer. In some embodiments, the method comprises administering to a subject an effective amount of a composition comprising an antibody or antigen-binding fragment thereof that specifically binds to PSMA. In some embodiments, the method comprises administering to the subject an antibody or antigen-binding fragment thereof, bispecific antibody, or chimeric antigen receptor. In one embodiment, the method comprises administering to a subject an effective amount of a composition comprising an isolated nucleic acid encoding an antibody or antigen-binding fragment thereof, bispecific antibody, or chimeric antigen receptor. In one embodiment, the method comprises administering to a subject a cell modified to express an antibody or antigen-binding fragment thereof, bispecific antibody, or chimeric antigen receptor.

This disclosure further provides a method for detecting cancer in a subject. In some embodiments, the method comprises administering to the subject a targeted imaging agent comprising an antibody or antigen-binding fragment thereof that specifically binds to PSMA. In some embodiments, the antibody or antigen-binding fragment thereof can be conjugated to any imaging agent to provide for a targeted imaging agent used in various imaging modalities, including but not limited to PET, SPECT, MRI, or optical imaging.

### Antibodies

In one aspect, this disclosure provides an antibody or antigen-binding fragment thereof that specifically binds to an epitope in the extracellular portion of PSMA. In some embodiments, the extracellular portion of PSMA comprises the amino acid sequence of SEQ ID NO: 1.

The disclosed antibody or antigen-binding fragment thereof is based, in part, upon the optimization of anti-PSMA antibodies described in WO2017180713A1. The optimization comprises the mutation of a “NG” sequence into “SG” in CDR3 of a light chain. Importantly, such optimization abrogates the potential deamidation of asparagine (Asn or N). It also confers the disclosed antibody or antigen-binding fragment thereof with several superior properties, including improved stability and effectiveness, without a negative impact on antigen-binding affinity. For example, the affinity of the optimized full antibody, as represented by PSMAbLm, demonstrates the same or even slightly higher affinity than its parent antibody PSMAb (e.g., Kd=0.049 nM of PSMAbLm compared to Kd=0.1088 nM of the antibody of WO2017180713A1).

The disclosed antibody or antigen-binding fragment is not limited to the mutation of “NG” sequence into “SG” in CDR3 of a light chain. The disclosed antibody or antigen-binding fragment also comprises any possible mutations that contain one or combined mutations listed below: any mutation of Asn in CDR3 of the light chain, including but not limited to the mutation to Ala, Arg, Asp, Cys, Glu, Gln, Gly, His, Ile, Leu, Lys, Met, Phe, Pro, Ser, Thr Trp, Tyr, or Val; and/or any mutation of Gly in CDR3 of the light chain, including but not limited to the mutation to Ala, Arg, Asn, Asp, Cys, Glu, Gln, His, Ile, Leu, Lys, Met, Phe, Pro, Ser, Thr, Trp, Tyr, or Val.

The disclosed antibody or antigen-binding fragment also comprises any possible mutations that contain one or combined mutations listed below to remove the potential deamidation site of “NS” in the FR3 of the heavy chain: any mutation of Asn in FR3 of the heavy chain, including but not limited to the mutation to Ala, Arg, Asp, Cys, Glu, Gln, Gly, His, Ile, Leu, Lys, Met, Phe, Pro, Ser, Thr, Trp, Tyr, or Val; and/or any mutation of Ser in FR3 of the heavy chain, including but not limited to the mutation to Ala, Arg, Asn, Asp, Cys, Glu, Gln, Gly, His, Ile, Leu, Lys, Met, Phe, Pro, Thr, Trp, Tyr, or Val.

The disclosed antibody or antigen-binding fragment also comprises any possible mutations that contain one or combined mutations listed below to remove the potential deamidation site of “NT”: any mutation of Asn in CDR2 of the light chain, including but not limited to the mutation to Ala, Arg, Asp, Cys, Glu, Gln, Gly, His, Ile, Leu, Lys, Met, Phe, Pro, Ser, Thr Trp, Tyr, or Val; and/or any mutation of Thr in CDR2 of the light chain, including but not limited to the mutation to Ala, Arg, Asn, Asp, Cys, Glu, Gln, Gly, His, Ile, Leu, Lys, Met, Phe, Pro, Ser, Trp, Tyr, or Val.

The disclosed antibody or antigen-binding fragment also comprises any possible mutations that contain one or combined mutations listed below to remove the potential deamidation site of “NT” in the FR3 of the heavy chain: any mutation of Asn in FR3 of the heavy chain, including but not limited to the mutation to Ala, Arg, Asp, Cys, Glu, Gln, Gly, His, Ile, Leu, Lys, Met, Phe, Pro, Ser, Thr, Trp, Tyr, or Val; and/or any mutation of Thr in FR3 of the heavy chain, including but not limited to the mutation to Ala, Arg, Asn, Asp, Cys, Glu, Gln, Gly, His, Ile, Leu, Lys, Met, Phe, Pro, Ser, Trp, Tyr, or Val.

The disclosed antibody or antigen-binding fragment also comprises any possible mutations that contain one or combined mutations listed below to remove the potential isomerization motif of “DG” in the CDR2 of the heavy chain: any mutation of Asp in CDR2 of the heavy chain, including but not limited to the mutation to Ala, Arg, Asn, Cys, Glu, Gln, Gly, His, Ile, Leu, Lys, Met, Phe, Pro, Ser, Thr, Trp, Tyr, or Val; and/or any mutation of Gly in CDR2 of the heavy chain, including but not limited to the mutation to Ala, Arg, Asn, Asp, Cys, Glu, Gln, His, Ile, Leu, Lys, Met, Phe, Pro, Ser, Thr, Trp, Tyr, or Val.

The disclosed antibody or antigen-binding fragment also comprises any possible mutations that contain one or combined mutations listed below to remove the potential isomerization motif of “DS” in the CDR3 of the light chain: any mutation of Asp in CDR3 of the light chain, including but not limited to the mutation to Ala, Arg, Asn, Cys, Glu, Gln, Gly, His, Ile, Leu, Lys, Met, Phe, Pro, Ser, Thr, Trp, Tyr, or Val; and/or any mutation of Ser in CDR3 of the light chain, including but not limited to the mutation to Ala, Arg, Asn, Asp, Cys, Glu, Gln, Gly, His, Ile, Leu, Lys, Met, Phe, Pro, Thr, Trp, Tyr, or Val.

The disclosed antibody or antigen-binding fragment also comprises any possible mutations that contain one or combined mutations listed below to remove the potential isomerization motif of “DS” in FR3 of the heavy chain: any mutation of Asp FR3 of the heavy chain, including but not limited to the mutation to Ala, Arg, Asn, Cys, Glu, Gln, Gly, His, Ile, Leu, Lys, Met, Phe, Pro, Ser, Thr, Trp, Tyr, or Val; and/or any mutation of Ser in FR3 of the heavy chain, including but not limited to the mutation to Ala, Arg, Asn, Asp, Cys, Glu, Gln, Gly, His, Ile, Leu, Lys, Met, Phe, Pro, Thr, Trp, Tyr, or Val.

Also provided in this disclosure is an antibody-drug conjugate (ADC). ADCs are a platform strategy to arm antibodies for antigen-specific toxicity delivery. The rationale is to conjugate antibodies, usually tumor-targeting antibodies, with super-toxic drugs to selectively kill tumor cells in a targeted manner while normal tissue is spared. Drugs used in ADC mainly fall into two types; one is microtubulin inhibitor, such as auristatin (MMAE, MMAF) (Stephan, J. P., et al. (2008) Bioconjug Chem, 19(8): 1673-83; Younes, A., et al. (2010) N Engl J Med, 363(19): 1812-21; Okeley, N. M., et al. (2010) Clin Cancer Res, 16(3): 888-970) and maytansinoid (DM1 and DM4) (Lambert, J. M. et al. (2014). J Med Chem, 57(16): 6949-64; Bender, B., et al. (2014) AAPS J, 16(5): 994-1008; Raufi, A., A.S. Ebrahim et al. (2013) Cancer Manag Res, 5: 225-33); the other type is a double-strand DNA cleavage agent, such as calicheamicin. Due to the supertoxicity, certain drugs could not be used alone as chemotherapy agents; instead, they have to be conjugated with antibodies to reduce the side effects and improve the therapeutic efficacy.

Also within the scope of this disclosure is a bispecific or multi-specific antibody. Bispecific monoclonal antibody (BsMAb, BsAb) is another strategy to make powerful anti-tumor weapons using novel antibodies. BsAb is an artificial protein that is composed of fragments of two different monoclonal antibodies and consequently binds to two different types of antigen. The most widely used application of this approach is in cancer immunotherapy, where BsMAbs are engineered that simultaneously bind to a cytotoxic cell (using a receptor-like CD3) and a target like a tumor cell to be destroyed (Müller D et al. (2010) BioDrugs, 24(2):89-98; Chames P1 et al. (2009) MAbs, 1(6):539-47). Bispecific T-cell engagers (BiTEs) and Dual-Affinity Re-Targeting (DART) are examples of small fragment BsAb. Various bigger BsAbs were also developed, such as knob-in-hole IgG, CrossMab, TrioMab, DVD Ig (Kontermann R E et al. (2015) Drug Discov Today, 20(7):838-47). In BsAbs, one arm could be a T or NK cell activation antibody, such as an anti-CD3, or anti-CD16 antibody, and the other arm could be a tumor-targeting antibody; or both arms target different tumor markers for synergetic inhibition of tumor growth.

This disclosure further provides a composition comprising a chimeric antigen receptor (CAR). For example, in some embodiments, the composition is a cell genetically modified to express a CAR. For example, in some embodiments, T or NK cells are genetically engineered to produce CARs on their surface that allow the T or NK cells to recognize a specific protein (antigen) on tumor cells. scFv is the most commonly used receptor for such engineering and have been successfully used in the clinic for cancer treatment(Grupp S A et al. (2013) N Engl J Med, 368(16):1509-18; Porter D L et al. (2011) N Engl J Med, 365(8):725-33). The scFv is fused via a hinge and a transmembrane domain to an intracellular signaling domain. Such molecules result in activation of the T- or NK-cell in response to recognition by the scFv of its target. When T or NK cells express such a CAR, they recognize and kill target cells that express the target antigen. Several CARs have been developed against tumor-associated antigens, and adoptive transfer approaches using such CAR-expressing T cells are currently in a clinical trial for the treatment of various cancers.

In one embodiment, the antibody or antigen-binding fragment thereof comprises high affinity anti-PSMA scFv Mut-gy1, which comprises the amino acid sequence of SEQ ID NO: 3.

In one embodiment, the antibody or antigen-binding fragment thereof comprises a light chain comprising an amino acid sequence of SEQ ID NO: 5. In one embodiment, the antibody or antigen-binding fragment thereof comprises VL FR1 comprising the amino acid sequence of SEQ ID NO: 7. In one embodiment, the antibody or antigen-binding fragment thereof comprises a VL CDR1 comprising the amino acid sequence of SEQ ID NO: 9. In one embodiment, the antibody or antigen-binding fragment thereof comprises a VL FR2 comprising the amino acid sequence of SEQ ID NO: 11. In one embodiment, the antibody or antigen-binding fragment thereof comprises a VL CDR2 comprising the amino acid sequence of SEQ ID NO: 13. In one embodiment, the antibody or antigen-binding fragment thereof comprises a VL FR3 comprises the amino acid sequence of SEQ ID NO: 15. In one embodiment, the antibody or antigen-binding fragment thereof comprises a VL CDR3 that comprises the amino acid sequence of SEQ ID NO: 17. In one embodiment, the antibody or antigen-binding fragment thereof comprises a VL FR4 comprising the amino acid sequence of SEQ ID NO: 19.

In one embodiment, the antibody or antigen-binding fragment thereof comprises a heavy chain comprising the amino acid sequence of SEQ ID NO: 21. In one embodiment, the antibody or antigen-binding fragment thereof comprises a VH FR1 comprising the amino acid sequence of SEQ ID NO: 23. In one embodiment, the antibody or antigen-binding fragment thereof comprises a VH CDR1 comprising the amino acid sequence of SEQ ID NO: 25. In one embodiment, the antibody or antigen-binding fragment thereof comprises a VH FR2 comprising the amino acid sequence of SEQ ID NO: 27. In one embodiment, the antibody or antigen-binding fragment thereof comprises a VH CDR2 comprising the amino acid sequence of SEQ ID NO: 29. In one embodiment, the antibody or antigen-binding fragment thereof comprises a VH FR3 comprising the amino acid sequence of SEQ ID NO: 31. In one embodiment, the antibody or antigen-binding fragment thereof comprises a VH CDR3 comprising the amino acid sequence of SEQ ID NO: 33. In one embodiment, the antibody or antigen-binding fragment thereof comprises a VH FR4 comprising the amino acid sequence of SEQ ID NO: 35. In one embodiment, the antibody or antigen-binding fragment thereof comprises a scFv linker comprising the amino acid sequence of SEQ ID NO: 37.

For example, in one embodiment, the antibody or antigen-binding fragment thereof comprises the scFv denoted herein as Mut-gy1. In one embodiment, the antibody or antigen-binding fragment thereof comprises the amino acid sequence of SEQ ID NO: 3. In one embodiment, Mut-gy1 comprises a light chain comprising the amino acid sequence of SEQ ID NO: 5 and a heavy chain comprising the amino acid sequence of SEQ ID NO: 21. In one embodiment, Mut-gy1 comprises a VL FR1 comprising the amino acid sequence of SEQ ID NO: 7; a VL CDR1 comprising the amino acid sequence of SEQ ID NO: 9; a VL FR2 comprising the amino acid sequence of SEQ ID NO: 11; a VL CDR2 comprising the amino acid sequence of SEQ ID NO: 13; a VL FR3 comprising the amino acid sequence of SEQ ID NO: 15; a VL CDR3 comprising the amino acid sequence of SEQ ID NO: 17; a VL FR4 comprising the amino acid sequence of SEQ ID NO: 19; a VH FR1 comprising the amino acid sequence of SEQ ID NO: 23; a VH CDR1 comprising the amino acid sequence of SEQ ID NO: 25; a VH FR2 comprising the amino acid sequence of SEQ ID NO: 27; a VH CDR2 comprising the amino acid sequence of SEQ ID NO: 29; a VH FR3 comprising the amino acid sequence of SEQ ID NO: 31; a VH CDR3 comprising the amino acid sequence of SEQ ID NO: 33; and/or a VH FR4 comprising the amino acid sequence of SEQ ID NO: 35. In some embodiments, the antibody or antigen-binding fragment thereof comprises a scFv linker comprising the amino acid sequence of SEQ ID NO: 37.

In one embodiment, the antibody or antigen-binding fragment thereof comprises one or more mutations. For example, in one embodiment, the antibody or antigen-binding fragment thereof comprises a VL FR2 comprising the amino acid sequence of SEQ ID NO: 39, where SEQ ID NO: 39 comprises a V→A point mutation with respect to SEQ ID NO: 11. In one embodiment, the antibody or antigen-binding fragment thereof comprises a VL CDR2 comprising the amino acid sequence of SEQ ID NO: 41, where SEQ ID NO: 41 comprises a G→E point mutation with respect to SEQ ID NO: 13. In one embodiment, the antibody or antigen-binding fragment thereof comprises a VL FR4 comprising the amino acid sequence of SEQ ID NO: 43, where SEQ ID NO: 43 comprises a V→A point mutation with respect to SEQ ID NO: 19. In one embodiment, the antibody or antigen-binding fragment thereof comprises a VH CDR1 comprising the amino acid sequence of SEQ ID NO: 45, where SEQ ID NO: 45 comprises a S→F point mutation with respect to SEQ ID NO: 25. In one embodiment, the antibody or antigen-binding fragment thereof comprises a VH FR3 comprising the amino acid sequence of SEQ ID NO: 47, where SEQ ID NO: 47 comprises an I→V point mutation with respect to SEQ ID NO: 31. In one embodiment, the antibody or antigen-binding fragment thereof comprises a VH CDR3 comprising the amino acid sequence of SEQ ID NO: 49, where SEQ ID NO: 49 comprises a D→G point mutation with respect to SEQ ID NO: 33. In one embodiment, the antibody or antigen-binding fragment thereof comprises a VH FR4 comprising the amino acid sequence of SEQ ID NO: 51, where SEQ ID NO: 51 comprises a G→E point mutation with respect to SEQ ID NO: 35.

In one embodiment, the antibody or antigen-binding fragment thereof comprises a scFv denoted herein as Mut-gy 1-st. In one embodiment, Mut-gy1-st comprises a VL FR2 comprising the amino acid sequence of SEQ ID NO: 39; a VH CDR1 comprising the amino acid sequence of SEQ ID NO: 45, and a VH FR4 comprising the amino acid sequence of SEQ ID NO: 51. In one embodiment, Mut-gy 1-st comprises a VL FR1 comprising the amino acid sequence of SEQ ID NO: 7; a VL CDR1 comprising the amino acid sequence of SEQ ID NO: 9; a VL FR2 comprising the amino acid sequence of SEQ ID NO: 39; a VL CDR2 comprising the amino acid sequence of SEQ ID NO: 13; a VL FR3 comprising the amino acid sequence of SEQ ID NO: 15; a VL CDR3 comprising the amino acid sequence of SEQ ID NO: 17; a VL FR4 comprising the amino acid sequence of SEQ ID NO: 19; a VH FR1 comprising the amino acid sequence of SEQ ID NO: 23; a VH CDR1 comprising the amino acid sequence of SEQ ID NO: 45; a VH FR2 comprising the amino acid sequence of SEQ ID NO: 27; a VH CDR2 comprising the amino acid sequence of SEQ ID NO: 29; a VH FR3 comprising the amino acid sequence of SEQ ID NO: 31; a VH CDR3 comprising the amino acid sequence of SEQ ID NO: 33; and/or a VH FR4 comprising the amino acid sequence of SEQ ID NO: 51. In some embodiments, the antibody or antigen-binding fragment thereof comprises a scFv linker comprising the amino acid sequence of SEQ ID NO: 37.

In one embodiment, the composition comprises an antibody fragment comprising a scFv denoted herein as Mut-gy1-2. In one embodiment, Mut-gy1-2 comprises a VL CDR2 comprising the amino acid sequence of SEQ ID NO: 41; a VL FR4 comprising the amino acid sequence of SEQ ID NO: 43; a VH CDR1 comprising the amino acid sequence of SEQ ID NO: 45, and a VH FR3 comprising the amino acid sequence of SEQ ID NO: 47. In one embodiment, Mut-gy1-2 comprises a VL FR1 comprising the amino acid sequence of SEQ ID NO: 7; a VL CDR1 comprising the amino acid sequence of SEQ ID NO: 9; a VL FR2 comprising the amino acid sequence of SEQ ID NO: 11; a VL CDR2 comprising the amino acid sequence of SEQ ID NO: 41; a VL FR3 comprising the amino acid sequence of SEQ ID NO: 15; a VL CDR3 comprising the amino acid sequence of SEQ ID NO: 17; a VL FR4 comprising the amino acid sequence of SEQ ID NO: 43; a VH FR1 comprising the amino acid sequence of SEQ ID NO: 23 a VH CDR1 comprising the amino acid sequence of SEQ ID NO: 45; a VH FR2 comprising the amino acid sequence of SEQ ID NO: 27; a VH CDR2 comprising the amino acid sequence of SEQ ID NO: 29; a VH FR3 comprising the amino acid sequence of SEQ ID NO: 47; a VH CDR3 comprising the amino acid sequence of SEQ ID NO: 33; and/or a VH FR4 comprising the amino acid sequence of SEQ ID NO: 35. In some embodiments, the antibody or antigen-binding fragment thereof comprises a scFv linker comprising the amino acid sequence of SEQ ID NO: 37.

In one embodiment, the composition comprises an antibody fragment comprising a scFv denoted herein as Mut-gy1-3. In one embodiment, Mut-gy1-3 comprises a VH CDR1 comprising the amino acid sequence of SEQ ID NO: 45, and a VH CDR3 comprising the amino acid sequence of SEQ ID NO: 49. In one embodiment, Mut-gy1-3 comprises a VL FR1 comprising the amino acid sequence of SEQ ID NO: 7; a VL CDR1 comprising the amino acid sequence of SEQ ID NO: 9; a VL FR2 comprising the amino acid sequence of SEQ ID NO: 11; a VL CDR2 comprising the amino acid sequence of SEQ ID NO: 13; a VL FR3 comprising the amino acid sequence of SEQ ID NO: 15; a VL CDR3 comprising the amino acid sequence of SEQ ID NO: 17; a VL FR4 comprising the amino acid sequence of SEQ ID NO: 19; a VH FR1 comprising the amino acid sequence of SEQ ID NO: 23; a VH CDR1 comprising the amino acid sequence of SEQ ID NO: 45; a VH FR2 comprising the amino acid sequence of SEQ ID NO: 27; a VH CDR2 comprising the amino acid sequence of SEQ ID NO: 29; a VH FR3 comprising the amino acid sequence of SEQ ID NO: 31; a VH CDR3 comprising the amino acid sequence of SEQ ID NO: 49; and/or a VH FR4 comprising the amino acid sequence of SEQ ID NO: 35. In some embodiments, the antibody or antigen-binding fragment thereof comprises a scFv linker comprising the amino acid sequence of SEQ ID NO: 37.

In one embodiment, an scFv described herein can be engineered into another fragment antibody or full-length antibody, wherein fragment antibody refers to a Fab, Fab′, (Fab′)₂, Fv, scFv-Fc, scFv-CH2, scFv-CH3, or a full antibody.

In some embodiments, the antibody or antigen-binding fragment thereof comprises Mut-gy1-2 scFv, which is denoted herein as PSMAbLm. In one embodiment, the antibody or antigen-binding fragment thereof comprises a heavy chain comprising the amino acid sequence of SEQ ID NO: 68. In one embodiment, the antibody or antigen-binding fragment thereof comprises a heavy chain having a signal peptide. In some embodiments, the signal peptide comprises the amino acid sequence of SEQ ID NO: 53 or 55.

In one embodiment, the antibody or antigen-binding fragment thereof comprises a heavy chain variable region comprising the amino acid sequence of SEQ ID NO: 57. In one embodiment, the antibody comprises a heavy chain constant region of SEQ ID NO: 59.

In one embodiment, the antibody or antigen-binding fragment thereof comprises a light chain comprising the amino acid sequence of SEQ ID NO: 69. In one embodiment, the antibody or antigen-binding fragment thereof comprises a light chain having a signal peptide. In some embodiments, the signal peptide comprises the amino acid sequence of SEQ ID NO: 61 or 63.

In one embodiment, the antibody or antigen-binding fragment thereof y comprises a light chain variable region comprising the amino acid sequence of SEQ ID NO: 65. In one embodiment, the antibody or antigen-binding fragment thereof comprises a light chain constant region of SEQ ID NO: 67.

In one aspect, this disclosure provides an antibody or antigen-binding fragment thereof or a variant thereof that specifically binds to PSMA, such as human PSMA. In some embodiments, the antibody or antigen-binding fragment thereof comprises: (i) a light chain variable region (LCVR) comprising three light chain complementarity determining regions (LCDRs) (LCDR1, LCDR2, and LCDR3), wherein the LCDR1 comprises the amino acid sequence of SEQ ID NO: 9, the LCDR2 comprises the amino acid sequence of SEQ ID NO: 13 or 41, and the LCDR3 comprises the amino acid sequence of SEQ ID NO: 17; and/or (ii) heavy chain variable region (HCVR) three heavy chain complementarity determining regions (HCDRs) (HCDR1, HCDR2, and HCDR3), comprising wherein the HCDR1 comprises the amino acid sequence of SEQ ID NO: 25 or 45, the HCDR2 comprises the amino acid sequence of SEQ ID NO: 29, and the HCDR3 comprises the amino acid sequence of SEQ ID NO: 33 or 49, wherein the variant comprises one or more conservative amino acid substitutions in the LCDR1, the LCDR2, the LCDR3, the HCDR1, the HCDR2, or the HCDR3.

In some embodiments, the LCDR1 comprises the amino acid sequence of SEQ ID NO: 9, the LCDR2 comprises the amino acid sequence of SEQ ID NO: 13, the LCDR3 comprises the amino acid sequence of SEQ ID NO: 17; the HCDR1 comprises the amino acid sequence of SEQ ID NO: 25, the HCDR2 comprises the amino acid sequence of SEQ ID NO: 29, and the HCDR3 comprises the amino acid sequence of SEQ ID NO: 33.

In some embodiments, the LCDR1 comprises the amino acid sequence of SEQ ID NO: 9, the LCDR2 comprises the amino acid sequence of SEQ ID NO: 13, the LCDR3 comprises the amino acid sequence of SEQ ID NO: 17; the HCDR1 comprises the amino acid sequence of SEQ ID NO: 45, the HCDR2 comprises the amino acid sequence of SEQ ID NO: 29, and the HCDR3 comprises the amino acid sequence of SEQ ID NO: 33.

In some embodiments, the LCDR1 comprises the amino acid sequence of SEQ ID NO: 9, the LCDR2 comprises the amino acid sequence of SEQ ID NO: 41, the LCDR3 comprises the amino acid sequence of SEQ ID NO: 17; the HCDR1 comprises the amino acid sequence of SEQ ID NO: 45, the HCDR2 comprises the amino acid sequence of SEQ ID NO: 29, and the HCDR3 comprises the amino acid sequence of SEQ ID NO: 33.

In some embodiments, the LCDR1 comprises the amino acid sequence of SEQ ID NO: 9, the LCDR2 comprises the amino acid sequence of SEQ ID NO: 13, the LCDR3 comprises the amino acid sequence of SEQ ID NO: 17; the HCDR1 comprises the amino acid sequence of SEQ ID NO: 45, the HCDR2 comprises the amino acid sequence of SEQ ID NO: 29, and the HCDR3 comprises the amino acid sequence of SEQ ID NO: 49.

In some embodiments, the light chain variable region comprises one or more of the amino acid sequences of SEQ ID NOs: 5, 7, 11, 15, 19, 39, 43, 61, 63, 65, 67, and 69.

In some embodiments, the heavy chain variable region comprises one or more of the amino acid sequences of SEQ ID NOs: 21, 23, 27, 31, 35, 47, 51, 53, 55, 57, 59, and 68.

In some embodiments, the light chain variable region comprises an amino acid sequence with at least 75% (e.g., 75%, 80%, 85%, 90%, 91%, 92%, 93%, 94%, 95%, 96%, 97%, 98%, 99%) identity to the amino acid sequence of SEQ ID NO: 5, or comprises the amino acid sequence of SEQ ID NO: 5; and/or the heavy chain variable region comprises an amino acid sequence with at least 75% (e.g., 75%, 80%, 85%, 90%, 91%, 92%, 93%, 94%, 95%, 96%, 97%, 98%, 99%) identity to the amino acid sequence of SEQ ID NO: 21, or comprises the amino acid sequence of SEQ ID NO: 21.

In some embodiments, the light chain variable region comprises an amino acid sequence with at least 75% (e.g., 75%, 80%, 85%, 90%, 91%, 92%, 93%, 94%, 95%, 96%, 97%, 98%, 99%) identity to the amino acid sequence of SEQ ID NO: 70, or comprises the amino acid sequence of SEQ ID NO: 70; and/or the heavy chain variable region comprises an amino acid sequence with at least 75% (e.g., 75%, 80%, 85%, 90%, 91%, 92%, 93%, 94%, 95%, 96%, 97%, 98%, 99%) identity to the amino acid sequence of SEQ ID NO: 72, or comprises the amino acid sequence of SEQ ID NO: 72.

In some embodiments, the light chain variable region comprises an amino acid sequence with at least 75% (e.g., 75%, 80%, 85%, 90%, 91%, 92%, 93%, 94%, 95%, 96%, 97%, 98%, 99%) identity to the amino acid sequence of SEQ ID NO: 71, or comprises the amino acid sequence of SEQ ID NO: 71; and/or the heavy chain variable region comprises an amino acid sequence with at least 75% (e.g., 75%, 80%, 85%, 90%, 91%, 92%, 93%, 94%, 95%, 96%, 97%, 98%, 99%) identity to the amino acid sequence of SEQ ID NO: 73, or comprises the amino acid sequence of SEQ ID NO: 73.

In some embodiments, the light chain variable region comprises an amino acid sequence with at least 75% (e.g., 75%, 80%, 85%, 90%, 91%, 92%, 93%, 94%, 95%, 96%, 97%, 98%, 99%) identity to the amino acid sequence of SEQ ID NO: 5, or comprises the amino acid sequence of SEQ ID NO: 5; and/or the heavy chain variable region comprises an amino acid sequence with at least 75% (e.g., 75%, 80%, 85%, 90%, 91%, 92%, 93%, 94%, 95%, 96%, 97%, 98%, 99%) identity to the amino acid sequence of SEQ ID NO: 74, or comprises the amino acid sequence of SEQ ID NO: 74.

In some embodiments, the light chain variable region and the heavy chain variable region comprise a LCVR and HCVR amino acid sequence pair of SEQ ID NOs: 5 and 21, SEQ ID NOs: 70 and 72, SEQ ID NOs: 71 and 73, or SEQ ID NOs: 5 and 74.

In some embodiments, the antibody or antigen-binding fragment thereof or variant thereof comprises a heavy chain/light chain sequence pair of SEQ ID NOs: 75 and 77, SEQ ID NOs: 76 and 78, SEQ ID NOs: 69 and 68, or SEQ ID NOs: 75 and 79.

In some embodiments, the antibody or antigen-binding fragment thereof or variant thereof comprises at least one of the amino acid sequences of SEQ ID NOs: 3 and 37.

### Representative Sequences

| antibody | | | VL | | | | | | | VH | | | | | | |
|---|---|---|---|---|---|---|---|---|---|---|---|---|---|---|---|---|
| variants | VL | VH | CDR1 | CDR2 | CDR3 | FR1 | FR2 | FR3 | FR4 | CDR1 | CDR2 | CDR3 | FR1 | FR2 | FR3 | FR4 |
| Mut-gy1 | 5 | 21 | 9 | 13 | 17 | 7 | 11 | 15 | 19 | 25 | 29 | 33 | 23 | 27 | 31 | 35 |
| Mut-gy1-st | 70 | 72 | 9 | 13 | 17 | 7 | 39 | 15 | 19 | 45 | 29 | 33 | 23 | 27 | 31 | 51 |
| Mut-gy1-2 | 71 | 73 | 9 | 41 | 17 | 7 | 11 | 15 | 43 | 45 | 29 | 33 | 23 | 27 | 47 | 35 |
| Mut-gy1-3 | 5 | 74 | 9 | 13 | 17 | 7 | 11 | 15 | 19 | 45 | 29 | 49 | 23 | 27 | 31 | 35 |

| | | Light chain | | Heavy chain | |
|---|---|---|---|---|---|
| | Associated | signal | light | signal | heavy |
| Full Ab name | scFv | peptide | chain | peptide | chain |
| PSMAb1Lm | Mut-gy1 | 63 | 75 | 55 | 77 |
| PSMAb1stLm | Mut-gy1-st | 63 | 76 | 55 | 78 |
| PSMAbLm | Mut-gy1-2 | 63 | 69 | 55 | 68 |
| PSMAb3Lm | Mut-gy1-3 | 63 | 75 | 55 | 79 |

PSMA extracellular domain:
SEQ ID NO: 1
KSSNEATNITPKHNMKAFLDELKAENIKKFLYNFTQIPHLAGTEQNFQLAKQIQSQW
KEFGLDSVELAHYDVLLSYPNKTHPNYISIINEDGNEIENTSLFEPPPPGYENVSDIVPPES
AFSPQGMPEGDLVYVNYARTEDFFKLERDMKINCSGKIVIARYGKVERGNKVKNAQLAGAKG
VILYSDPADYFAPGVKSYPDGWNLPGGGVQRGNILNLNGAGDPLTPGYPANEYAYRRGIAEA
VGLPSIPVHPIGYYDAQKLLEKMGGSAPPDSSWRGSLKVPYNVGPGFTGNESTQKVKMHIHS
TNEVTRIYNVIGTLRGAVEPDRYVILGGHRDSWVEGGIDPQSGAAVVHEIVRSFGTLKKEGW
RPRRTILFASWDAEEFGLLGSTEWAEENSRLLQERGVAYINADSSIEGNYTLRVDCTPLMYS
LVHNLTKELKSPDEGFEGKSLYESWTKKSPSPEFSGMPRISKLGSGNDFEVFFQRLGIASGR
ARYTKNWETNKFSGYPLYHSVYETYELVEKFYDPMFKYHLTVAQVRGGMVFELANSIVLPED
CRDYAVVLRKYADKIYSISMKHPQEMKTYSVSFDSLFSAVKNFTEIASKFSERLQDEDKSNP
IVLRMMNDQLMFLERAFIDPLGLPDRPFYRHVIYAPSSHNKYAGESFPGIYDALFDIESKVD
PSKAWGEVKRQIYVAAFTVQAAAETLSEVA
Mut-gy1 scFv nucleotide sequence:
SEQ ID NO: 2
CAGTCTGTGCTGACTCAGCCGCCCTCAGTGTCTGGGGCCCCAGGGCAGAGTGTCATT
ATCTCCTGCACTGGGAGCAGCTCCAACATCGGGGCAGGTTCTCATGTACACTGGTACCAGCA
GGTTCCAGGAACAGCCCCCAAACTCCTCATCTATGGAAACACCAATCGGCCCTCAGGGGTCC
CTGACCGATTCTCTGGCTCCAAGTCTGGCACCTCAGGTTCCCTGGCCATCACTGGACTCCAG
CCTGAGGATGAGGCTGATTATTATTGTGCAACATGGGATGACAGTCTGAGTGGTGTAATATT
CGGCGGAGGGACCAAGGTCACCGTCCTAGGCGGATCCTCTAGGTCAAGTTCCAGCGGCGGCG
GTGGCAGCGGAGGCGGCGGTGAGGTGCAGCTGGTGGAGTCTGGGGGAGCCCTGGCCAAGCCT
GGGGGGTCCCTGAGACTCTCCTGTGCAGCCTCTGGATCCACCCTCAGTGGCTATGCTATGCA
CTGGGTCCGCCAGGCTCCAGGCAAGGGGCTGGAGTGGGTGGCAGTTATATCATATGATGGAA
GCAATAAATACTACGCAGACTCCGTGAAGGGCCGATTCACCATCTCCAGAGACAATTCCAAG
AACACGCTGTTTCTGCAAATGAACAGCCTGAGACCTGAGGACACGGCTGTGTACTATTGTGC
TAAAGGCCTTACTTGGGGACTCGGTGACAATGATGCTCTCGATATCTGGGGCCCCGGGACCA
CGGTCACCGTCTCCTCA
Mut-gy1 scFv amino acid sequence:
SEQ ID NO: 3
QSVLTQPPSVSGAPGQSVIISCTGSSSNIGAGSHVHWYQQVPGTAPKLLIYGNTNRP
SGVPDRESGSKSGTSGSLAITGLQPEDEADYYCATWDDSLSGVIFGGGTKVTVLGGSSRSSS
SGGGGSGGGGEVQLVESGGALAKPGGSLRLSCAASGSTLSGYAMHWVRQAPGKGLEWVAVIS
YDGSNKYYADSVKGRFTISRDNSKNTLFLQMNSLRPEDTAVYYCAKGLTWGLGDNDALDIWG
PGTTVTVSS
Mut-gy1 VL nucleotide sequence:
SEQ ID NO: 4
CAGTCTGTGCTGACTCAGCCGCCCTCAGTGTCTGGGGCCCCAGGGCAGAGTGTCATT
ATCTCCTGCACTGGGAGCAGCTCCAACATCGGGGCAGGTTCTCATGTACACTGGTACCAGCA
GGTTCCAGGAACAGCCCCCAAACTCCTCATCTATGGAAACACCAATCGGCCCTCAGGGGTCC
CTGACCGATTCTCTGGCTCCAAGTCTGGCACCTCAGGTTCCCTGGCCATCACTGGACTCCAG
CCTGAGGATGAGGCTGATTATTATTGTGCAACATGGGATGACAGTCTGAGTGGTGTAATATT
CGGCGGAGGGACCAAGGTCACCGTCCTA
Mut-gy1 VL amino acid sequence:
SEQ ID NO: 5
QSVLTQPPSVSGAPGQSVIISCTGSSSNIGAGSHVHWYQQVPGTAPKLLIYGNTNRP
SGVPDRFSGSKSGTSGSLAITGLQPEDEADYYCATWDDSLSGVIFGGGTKVTVL
Mut-gy1 VL FR1 nucleotide sequence:
SEQ ID NO: 6
CAGTCTGTGCTGACTCAGCCGCCCTCAGTGTCTGGGGCCCCAGGGCAGAGTGTCATT
ATCTCCTGCACTGGGAGC
Mut-gy1 VL FR1 amino acid sequence:
SEQ ID NO: 7
QSVLTQPPSVSGAPGQSVIISCTGS
Mut-gy1 VL CDR1 nucleotide sequence:
SEQ ID NO: 8
AGCTCCAACATCGGGGCAGGTTCTCAT
Mut-gy1 VL CDR1 amino acid sequence:
SEQ ID NO: 9
SSNIGAGSH
Mut-gy1 VL FR2 nucleotide sequence:
SEQ ID NO: 10
GTACACTGGTACCAGCAGGTTCCAGGAACAGCCCCCAAACTCCTCATCTAT
Mut-gy1 VL FR2 amino acid sequence:
SEQ ID NO: 11
VHWYQQVPGTAPKLLIY
Mut-gy1 VL CDR2 nucleotide sequence:
SEQ ID NO: 12
GGAAACACC
Mut-gy1 VL CDR2 amino acid sequence:
SEQ ID NO: 13
GNT
Mut-gy1 VL FR3 nucleotide sequence:
SEQ ID NO: 14
AATCGGCCCTCAGGGGTCCCTGACCGATTCTCTGGCTCCAAGTCTGGCACCTCAGGT
TCCCTGGCCATCACTGGACTCCAGCCTGAGGATGAGGCTGATTATTATTGT
Mut-gy1 VL FR3 amino acid sequence:
SEQ ID NO: 15
NRPSGVPDRESGSKSGTSGSLAITGLQPEDEADYYC
Mut-gy1 VL CDR3 nucleotide sequence:
SEQ ID NO: 16
GCAACATGGGATGACAGTCTGAGTGGTGTAATA
Mut-gy1 VL CDR3 amino acid sequence
SEQ ID NO: 17
ATWDDSLSGVI
(Asn to Ser substitution is bolded)
SEQ ID NO: 18: Mut-gy1 VL FR4 nucleotide sequence
TTCGGCGGAGGGACCAAGGTCACCGTCCTA
Mut-gy1 VL FR4 amino acid sequence:
SEQ ID NO: 19
FGGGTKVTVL
Mut-gy1 VH nucleotide sequence:
SEQ ID NO: 20
GAGGTGCAGCTGGTGGAGTCTGGGGGAGCCCTGGCCAAGCCTGGGGGGTCCCTGAGA
CTCTCCTGTGCAGCCTCTGGATCCACCCTCAGTGGCTATGCTATGCACTGGGTCCGCCAGGC
TCCAGGCAAGGGGCTGGAGTGGGTGGCAGTTATATCATATGATGGAAGCAATAAATACTACG
CAGACTCCGTGAAGGGCCGATTCACCATCTCCAGAGACAATTCCAAGAACACGCTGTTTCTG
CAAATGAACAGCCTGAGACCTGAGGACACGGCTGTGTACTATTGTGCTAAAGGCCTTACTTG
GGGACTCGGTGACAATGATGCTCTCGATATCTGGGGCCCCGGGACCACGGTCACCGTCTCCT
CA
Mut-gy1 VH amino acid sequence:
SEQ ID NO: 21
EVQLVESGGALAKPGGSLRLSCAASGSTLSGYAMHWVRQAPGKGLEWVAVISYDGSN
KYYADSVKGRFTISRDNSKNTLFLQMNSLRPEDTAVYYCAKGLTWGLGDNDALDIWGPGTTV
TVSS
Mut-gy1 VH FR1 nucleotide sequence:
SEQ ID NO: 22
GAGGTGCAGCTGGTGGAGTCTGGGGGAGCCCTGGCCAAGCCTGGGGGGTCCCTGAGA
CTCTCCTGTGCAGCCTCT
Mut-gy1 VH FR1 amino acid sequence:
SEQ ID NO: 23
EVQLVESGGALAKPGGSLRLSCAAS
Mut-gy1 VH CDR1 nucleotide sequence:
SEQ ID NO: 24
GGATCCACCCTCAGTGGCTATGCT
Mut-gy1 VH CDR1 amino acid sequence:
SEQ ID NO: 25
GSTLSGYA
Mut-gy1 VH FR2 nucleotide sequence:
SEQ ID NO: 26
ATGCACTGGGTCCGCCAGGCTCCAGGCAAGGGGCTGGAGTGGGTGGCAGTT
Mut-gy1 VH FR2 amino acid sequence:
SEQ ID NO: 27
MHWVRQAPGKGLEWVAV
Mut-gy1 VH CDR2 nucleotide sequence:
SEQ ID NO: 28
ATATCATATGATGGAAGCAATAAA
Mut-gy1 VH CDR2 amino acid sequence:
SEQ ID NO: 29
ISYDGSNK
Mut-gy1 VH FR3 nucleotide sequence:
SEQ ID NO: 30
TACTACGCAGACTCCGTGAAGGGCCGATTCACCATCTCCAGAGACAATTCCAAGAAC
ACGCTGTTTCTGCAAATGAACAGCCTGAGACCTGAGGACACGGCTGTGTACTATTGT
Mut-gy1 VH FR3 amino acid sequence:
SEQ ID NO: 31
YYADSVKGRFTISRDNSKNTLFLQMNSLRPEDTAVYYC
Mut-gy1 VH CDR3 nucleotide sequence:
SEQ ID NO 32
GCTAAAGGCCTTACTTGGGGACTCGGTGACAATGATGCTCTCGATATC
Mut-gy1 VH CDR3 amino acid sequence:
SEQ ID NO: 33
AKGLTWGLGDNDALDI
Mut-gy1 VH FR4 nucleotide sequence:
SEQ ID NO: 34
TGGGGCCCCGGGACCACGGTCACCGTCTCCTCA
Mut-gy1 VH FR4 amino acid sequence:
SEQ ID NO: 35
WGPGTTVTVSS
Mut-gy1 scFv linker nucleotide sequence:
SEQ ID NO: 36
GGCGGATCCTCTAGGTCAAGTTCCAGCGGCGGCGGTGGCAGCGGAGGCGGCGGT
Mut-gy1 scFv linker amino acid sequence:
SEQ ID NO: 37
GGSSRSSSSGGGGSGGGG
Mut-gy1 VL FR2 nucleotide sequence (variant):
SEQ ID NO: 38
GTACACTGGTACCAGCAGGCTCCAGGAACAGCCCCCAAACTCCTCATCTAT
Mut-gy1 VL FR2 amino acid sequence (variant):
SEQ ID NO: 39
VHWYQQAPGTAPKLLIY (V = >A)
Mut-gy1 VL CDR2 nucleotide sequence (variant):
SEQ ID NO: 40
GAAAACACC
Mut-gy1 VL CDR2 amino acid sequence (variant):
SEQ ID NO: 41
ENT (G = >E)
Mut-gy1 VL FR4 nucleotide sequence (variant)
SEQ ID NO: 42
TTCGGCGGAGGGACCAAGGCCACCGTCCTA
Mut-gy1 VL FR4 amino acid sequence (variant):
SEQ ID NO: 43
FGGGTKATVL (V = >A)
Mut-gy1 VH CDR1 nucleotide sequence (variant):
SEQ ID NO: 44
GGATTCACCCTCAGTGGCTATGCT
Mut-gy1 VH CDR1 amino acid sequence (variant):
SEQ ID NO: 45
GFTLSGYA (S = >F)
SEQ ID NO: 46: Mut-gy1 VH FR3 nucleotide sequence (variant)
TACTACGCAGACTCCGTGAAGGGCCGATTCACCGTCTCCAGAGACAATTCCAAGAAC
ACGCTGTTTCTGCAAATGAACAGCCTGAGACCTGAGGACACGGCTGTGTACTATTGT
Mut-gy1 VH FR3 amino acid sequence (variant):
SEQ ID NO: 47
YYADSVKGRFTVSRDNSKNTLFLQMNSLRPEDTAVYYC (I = >V)
Mut-gy1 VH CDR3 nucleotide sequence (variant):
SEQ ID NO: 48
GCTAAAGGCCTTACTTGGGGACTCGGTGACAATGATGCTCTCGGTATC
Mut-gy1 VH CDR3 amino acid sequence (variant):
SEQ ID NO: 49
AKGLTWGLGDNDALGI (D = >G)
SEQ ID NO: 50: Mut-gy1 VH FR4 nucleotide sequence (variant):
TGGGGCCCCGAGACCACGGTCACCGTCTCCTCA
Mut-gy1 VH FR4 amino acid sequence (variant):
SEQ ID NO: 51
WGPETTVTVSS (G = >E)
PSMAbLm heavy chain nucleotide sequence:
SEQ ID NO: 52
ATGGAGTTTGGGCTGAGCTGGGTTTTCCTCGTTGCTCTTTTAAGAGGTGTCCAGTGT
GAGGTGCAGCTGGTGGAGTCTGGGGGAGCCCTGGCCAAGCCTGGGGGGTCCCTGAGACTCTC
CTGTGCAGCCTCTGGATTCACCCTCAGTGGCTATGCTATGCACTGGGTCCGCCAGGCTCCAG
GCAAGGGGCTGGAGTGGGTGGCAGTTATATCATATGATGGAAGCAATAAATACTACGCAGAC
TCCGTGAAGGGCCGATTCACCGTCTCCAGAGACAATTCCAAGAACACGCTGTTTCTGCAAAT
GAACAGCCTGAGACCTGAGGACACGGCTGTGTACTATTGTGCTAAAGGCCTTACCTGGGGAC
TCGGTGACAATGATGCTCTCGATATCTGGGGCCCCGGGACCACGGTCACCGTCTCCTCAGCC
TCCACCAAGGGCCCATCGGTCTTCCCCCTGGCACCCTCCTCCAAGAGCACCTCTGGGGGCAC
AGCGGCCCTGGGCTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAA
CGTGAATCACAAGCCCAGCAACACCAAGGTGGACAAGAAAGTTGAGCCCAAATCTTGTGACA
AAACTCACACATGCCCACCGTGCCCAGCACCTGAACTCCTGGGGGGACCGTCAGTCTTCCTC
TTCCCCCCAAAACCCAAGGACACCCTCATGATCTCCCGGACCCCTGAGGTCACATGCGTGGT
GGTGGACGTGAGCCACGAAGACCCTGAGGTCAAGTTCAACTGGTACGTGGACGGCGTGGAGG
TGCATAATGCCAAGACAAAGCCGCGGGAGGAGCAGTACAACAGCACGTACCGGGTGGTCAGC
GTCCTCACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCGAGAAC
CACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCC
GGAGAACAACTACAAGACCACGCCTCCCGTGCTGGACTCCGACGGCTCCTTCTTCCTCTACA
GCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGTAAATGA
PSMAbLm amino acid sequence:
SEQ ID NO: 53
MEFGLSWVFLVALLRGVQCEVQLVESGGALAKPGGSLRLSCAASGFTLSGYAMHWVR
QAPGKGLEWVAVISYDGSNKYYADSVKGRFTVSRDNSKNTLFLQMNSLRPEDTAVYYCAKGL
TWGLGDNDALDIWGPGTTVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKENWYVD
GVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
PSMAbLm heavy chain signal peptide nucleotide sequence:
SEQ ID NO: 54
ATGGAGTTTGGGCTGAGCTGGGTTTTCCTCGTTGCTCTTTTAAGAGGTGTCCAGTGT
PSMAbLm heavy chain signal peptide amino acid sequence:
SEQ ID NO: 55
MEFGLSWVELVALLRGVQC
PSMAbLm heavy chain variable region nucleotide sequence:
SEQ ID NO: 56
GAGGTGCAGCTGGTGGAGTCTGGGGGAGCCCTGGCCAAGCCTGGGGGGTCCCTGAGA
CTCTCCTGTGCAGCCTCTGGATTCACCCTCAGTGGCTATGCTATGCACTGGGTCCGCCAGGC
TCCAGGCAAGGGGCTGGAGTGGGTGGCAGTTATATCATATGATGGAAGCAATAAATACTACG
CAGACTCCGTGAAGGGCCGATTCACCGTCTCCAGAGACAATTCCAAGAACACGCTGTTTCTG
CAAATGAACAGCCTGAGACCTGAGGACACGGCTGTGTACTATTGTGCTAAAGGCCTTACCTG
GGGACTCGGTGACAATGATGCTCTCGATATCTGGGGCCCCGGGACCACGGTCACCGTCTCCT
CA
PSMAbLm heavy chain variable region amino acid sequence:
SEQ ID NO: 57
EVQLVESGGALAKPGGSLRLSCAASGFTLSGYAMHWVRQAPGKGLEWVAVISYDGSN
KYYADSVKGRFTVSRDNSKNTLFLQMNSLRPEDTAVYYCAKGLTWGLGDNDALDIWGPGTTV
TVSS
PSMAbLm heavy chain constant region nucleotide sequence:
SEQ ID NO: 58
GCCTCCACCAAGGGCCCATCGGTCTTCCCCCTGGCACCCTCCTCCAAGAGCACCTCT
GGGGGCACAGCGGCCCTGGGCTGCCTGGTCAAGGACTACTTCCCCGAACCGGTGACGGTGTC
GTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAATC
TTGTGACAAAACTCACACATGCCCACCGTGCCCAGCACCTGAACTCCTGGGGGGACCGTCAG
TCTTCCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGGG
TGGTCAGCGTCCTCACCGTCCTGCACCAGGACTGGCTGAATGGCAAGGAGTACAAGTGCAAG
GTCTCCAACAAAGCCCTCCCAGCCCCCATCGAGAAAACCATCTCCAAAGCCAAAGGGCAGCC
CCGAGAACCACAGGTGTACACCCTGCCCCCATCCCGGGATGAGCTGACCAAGAACCAGGTCA
GCCTGACCTGCCTGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGACGGCTCCTTCTT
CCTCTACAGCAAGCTCACCGTGGACAAGAGCAGGTGGCAGCAGGGGAACGTCTTCTCATGCT
CCGTGATGCATGAGGCTCTGCACAACCACTACACGCAGAAGAGCCTCTCCCTGTCTCCGGGT
AAA
PSMAbLm heavy chain constant region amino acid sequence:
SEQ ID NO: 59
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPELLG
GPSVFLFPPKPKDTLMISRTPEVTCVVVDVSHEDPEVKENWYVDGVEVHNAKTKPREEQYNS
TYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNV
FSCSVMHEALHNHYTQKSLSLSPGK
PSMAbLm light chain nucleotide sequence:
SEQ ID NO: 60
ATGGCCTGGTCTCCTCTCCTCCTCACTCTCCTCGCTCACTGCACAGGGTCCTGGGCC
CAGTCTGTGCTGACTCAGCCGCCCTCAGTGTCTGGGGCCCCAGGGCAGAGTGTCATTATCTC
CTGCACTGGGAGCAGCTCCAACATCGGGGCAGGTTCTCATGTACACTGGTACCAGCAGGTTC
CAGGAACAGCCCCCAAACTCCTCATCTATGAAAACACCAATCGGCCCTCAGGGGTCCCTGAC
CGATTCTCTGGCTCCAAGTCTGGCACCTCAGGTTCCCTGGCCATCACTGGACTCCAGCCTGA
GGATGAGGCTGATTATTATTGTGCAACATGGGATGACAGTCTGAGTGGTGTAATATTCGGCG
GAGGGACCAAGGCCACCGTCCTAGGTCAGCCCAAGGCTGCCCCCTCGGTCACTCTGTTCCCG
CCCTCCTCTGAGGAGCTTCAAGCCAACAAGGCCACACTGGTGTGTCTCATAAGTGACTTCTA
CCCGGGAGCCGTGACAGTGGCCTGGAAGGCAGATAGCAGCCCCGTCAAGGCGGGAGTGGAGA
CCACCACACCCTCCAAACAAAGCAACAACAAGTACGCGGCCAGCAGCTATCTGAGCCTGACG
CCTGAGCAGTGGAAGTCCCACAGAAGCTACAGCTGCCAGGTCACGCATGAAGGGAGCACCGT
GGAGAAGACAGTGGCCCCTACAGAATGTTCATGA
PSMAbLm light chain amino acid sequence:
SEQ ID NO: 61
MAWSPLLLTLLAHCTGSWAQSVLTQPPSVSGAPGQSVIISCTGSSSNIGAGSHVHWY
QQVPGTAPKLLIYENTNRPSGVPDRESGSKSGTSGSLAITGLQPEDEADYYCATWDDSLSGV
IFGGGTKATVLGQPKAAPSVTLEPPSSEELQANKATLVCLISDFYPGAVTVAWKADSSPVKA
GVETTTPSKQSNNKYAASSYLSLTPEQWKSHRSYSCQVTHEGSTVEKTVAPTECS
PSMAbLm light chain signal peptide nucleotide sequence:
SEQ ID NO: 62
ATGGCCTGGTCTCCTCTCCTCCTCACTCTCCTCGCTCACTGCACAGGGTCCTGGGCC
PSMAbLm light chain signal peptide amino acid sequence:
SEQ ID NO: 63
MAWSPLLLTLLAHCTGSWA
PSMAbLm light chain variable region nucleotide sequence:
SEQ ID NO: 64
CAGTCTGTGCTGACTCAGCCGCCCTCAGTGTCTGGGGCCCCAGGGCAGAGTGTCATT
ATCTCCTGCACTGGGAGCAGCTCCAACATCGGGGCAGGTTCTCATGTACACTGGTACCAGCA
GGTTCCAGGAACAGCCCCCAAACTCCTCATCTATGAAAACACCAATCGGCCCTCAGGGGTCC
CTGACCGATTCTCTGGCTCCAAGTCTGGCACCTCAGGTTCCCTGGCCATCACTGGACTCCAG
CCTGAGGATGAGGCTGATTATTATTGTGCAACATGGGATGACAGTCTGAGTGGTGTAATATT
CGGCGGAGGGACCAAGGCCACCGTCCTA
PSMAbLm light chain variable region amino acid sequence:
SEQ ID NO: 65
QSVLTQPPSVSGAPGQSVIISCTGSSSNIGAGSHVHWYQQVPGTAPKLLIYENTNRP
SGVPDRFSGSKSGTSGSLAITGLQPEDEADYYCATWDDSLSGVIFGGGTKATVL
PSMAbLm light chain constant region nucleotide sequence:
SEQ ID NO: 66
GGTCAGCCCAAGGCTGCCCCCTCGGTCACTCTGTTCCCGCCCTCCTCTGAGGAGCTT
CAAGCCAACAAGGCCACACTGGTGTGTCTCATAAGTGACTTCTACCCGGGAGCCGTGACAGT
GGCCTGGAAGGCAGATAGCAGCCCCGTCAAGGCGGGAGTGGAGACCACCACACCCTCCAAAC
AAAGCAACAACAAGTACGCGGCCAGCAGCTATCTGAGCCTGACGCCTGAGCAGTGGAAGTCC
CACAGAAGCTACAGCTGCCAGGTCACGCATGAAGGGAGCACCGTGGAGAAGACAGTGGCCCC
TACAGAATGTTCA
PSMAbLm light chain constant region amino acid sequence:
SEQ ID NO: 67
GQPKAAPSVTLEPPSSEELQANKATLVCLISDFYPGAVTVAWKADSSPVKAGVETTT
PSKQSNNKYAASSYLSLTPEQWKSHRSYSCQVTHEGSTVEKTVAPTECS
PSMAbLm heavy chain amino acid sequence without signal peptide:
SEQ ID NO: 68
EVQLVESGGALAKPGGSLRLSCAASGFTLSGYAMHWVRQAPGKGLEWVAVISYDGSN
KYYADSVKGRFTVSRDNSKNTLFLQMNSLRPEDTAVYYCAKGLTWGLGDNDALDIWGPGTTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKENWYVDGVEVHNAKTKPREEQYNST
YRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVE
SCSVMHEALHNHYTQKSLSLSPGK
PSMAbLm light chain amino acid sequence without signal peptide:
SEQ ID NO: 69
QSVLTQPPSVSGAPGQSVIISCTGSSSNIGAGSHVHWYQQVPGTAPKLLIYENTNRP
SGVPDRFSGSKSGTSGSLAITGLQPEDEADYYCATWDDSLSGVIFGGGTKATVLGQPKAAPS
VTLFPPSSEELQANKATLVCLISDFYPGAVTVAWKADSSPVKAGVETTTPSKQSNNKYAASS
YLSLTPEQWKSHRSYSCQVTHEGSTVEKTVAPTECS
Mut-gy1-st VL amino acid sequence:
SEQ ID NO: 70
QSVLTQPPSVSGAPGQSVIISCTGSSSNIGAGSHVHWYQQAPGTAPKLLIYGNTNRP
SGVPDRFSGSKSGTSGSLAITGLQPEDEADYYCATWDDSLSGVIFGGGTKVTVL
Mut-gy1-2 VL amino acid sequence:
SEQ ID NO: 71
QSVLTQPPSVSGAPGQSVIISCTGSSSNIGAGSHVHWYQQVPGTAPKLLIYENTNRP
SGVPDRESGSKSGTSGSLAITGLQPEDEADYYCATWDDSLSGVIFGGGTKATVL
Mut-gy1-st VH amino acid sequence:
SEQ ID NO: 72
EVQLVESGGALAKPGGSLRLSCAASGFTLSGYAMHWVRQAPGKGLEWVAVISYDGSN
KYYADSVKGRFTISRDNSKNTLFLQMNSLRPEDTAVYYCAKGLTWGLGDNDALDIWGPETTV
TVSS
Mut-gy1-2 VH amino acid sequence:
SEQ ID NO: 73
EVQLVESGGALAKPGGSLRLSCAASGFTLSGYAMHWVRQAPGKGLEWVAVISYDGSN
KYYADSVKGRFTVSRDNSKNTLFLQMNSLRPEDTAVYYCAKGLTWGLGDNDALDIWGPGTTV
TVSS
Mut-gy1-3 VH amino acid sequence:
SEQ ID NO: 74
EVQLVESGGALAKPGGSLRLSCAASGFTLSGYAMHWVRQAPGKGLEWVAVISYDGSN
KYYADSVKGRFTISRDNSKNTLFLQMNSLRPEDTAVYYCAKGLTWGLGDNDALGIWGPGTTV
TVSS
PSMAb1Lm light chain amino acid sequence without signal peptide
(from Mut-gy1):
SEQ ID NO: 75
QSVLTQPPSVSGAPGQSVIISCTGSSSNIGAGSHVHWYQQVPGTAPKLLIYGNTNRP
SGVPDRFSGSKSGTSGSLAITGLQPEDEADYYCATWDDSLSGVIFGGGTKVTVLGQPKAAPS
VTLFPPSSEELQANKATLVCLISDFYPGAVTVAWKADSSPVKAGVETTTPSKQSNNKYAASS
YLSLTPEQWKSHRSYSCQVTHEGSTVEKTVAPTECS
PSMAb1stLm light chain amino acid sequence without signal peptide
(from Mut-gy1-st):
SEQ ID NO: 76
QSVLTQPPSVSGAPGQSVIISCTGSSSNIGAGSHVHWYQQAPGTAPKLLIYGNTNRP
SGVPDRFSGSKSGTSGSLAITGLQPEDEADYYCATWDDSLSGVIFGGGTKVTVLGQPKAAPS
VTLFPPSSEELQANKATLVCLISDFYPGAVTVAWKADSSPVKAGVETTTPSKQSNNKYAASS
YLSLTPEQWKSHRSYSCQVTHEGSTVEKTVAPTECS
PSMAb1Lm heavy chain amino acid sequence without signal peptide
(from Mut-gy1):
SEQ ID NO: 77
EVQLVESGGALAKPGGSLRLSCAASGSTLSGYAMHWVRQAPGKGLEWVAVISYDGSN
KYYADSVKGRFTISRDNSKNTLFLQMNSLRPEDTAVYYCAKGLTWGLGDNDALDIWGPGTTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKENWYVDGVEVHNAKTKPREEQYNST
YRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVE
SCSVMHEALHNHYTQKSLSLSPGK
PSMAb1stLm heavy chain amino acid sequence without signal peptide
(from Mut-gy1-st):
SEQ ID NO: 78
EVQLVESGGALAKPGGSLRLSCAASGFTLSGYAMHWVRQAPGKGLEWVAVISYDGSN
KYYADSVKGRFTISRDNSKNTLFLQMNSLRPEDTAVYYCAKGLTWGLGDNDALDIWGPETTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKENWYVDGVEVHNAKTKPREEQYNST
YRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVF
SCSVMHEALHNHYTQKSLSLSPGK
PSMAb3Lm light chain amino acid sequence without signal peptide
(from Mut-gy1-3):
SEQ ID NO: 79
EVQLVESGGALAKPGGSLRLSCAASGFTLSGYAMHWVRQAPGKGLEWVAVISYDGSN
KYYADSVKGRFTISRDNSKNTLFLQMNSLRPEDTAVYYCAKGLTWGLGDNDALGIWGPGTTV
TVSSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVE
SCSVMHEALHNHYTQKSLSLSPGK

In some embodiments, the term “epitope” refers to a region of the antigen that binds to the antibody. It is the region of an antigen recognized by a first antibody wherein the binding of the first antibody to the region prevents binding of a second antibody or other bivalent molecule to the region. The region encompasses a particular core sequence or sequences selectively recognized by a class of antibodies. In general, epitopes are composed of local surface structures that can be formed by contiguous or noncontiguous amino acid sequences.

In another embodiment, the term “selectively recognizes,” “selectively binds,” or “selectively recognizes” means that binding of the antibody or other bivalent molecule to an epitope is at least 2-fold greater, 2-5 fold greater, or more than 5-fold greater than the binding of the bivalent molecule to an unrelated epitope or than the binding of an unrelated bivalent molecule to the epitope, as determined by techniques known in the art and described herein, such as, for example, ELISA and cold displacement assays.

In some embodiments, the term “antibody” refers to the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains VL and CL, and each heavy chain comprising immunoglobulin domains VH, Cγ1, Cγ2, Cγ₃, and Cγ4. In each pair, the light and heavy chain variable regions (VL and VH) are together responsible for binding to an antigen, and the constant regions (CL, Cγ1, Cγ2, Cγ3, and Cγ4, particularly Cγ1, Cγ2, and Cγ3) are responsible for antibody effector functions. In some mammals, for example, in camels and llamas, full-length antibodies may consist of only two heavy chains, each heavy chain comprising immunoglobulin domains VH, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains, including but not limited to VH, Cγ1, Cγ2, Cγ3, Cγ4, VL, and CL.

Depending on the amino acid sequence of the constant domain of their heavy chains, intact antibodies can be assigned to different “classes.” There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

In some embodiments, the term “antibody” or “antigen-binding fragment” respectively refer to intact molecules as well as functional fragments thereof, such as Fab, a scFv-Fc bivalent molecule, F(ab′)2, and Fv that are capable of specifically interacting with a desired target. In some embodiments, the antigen-binding fragments comprise:
(1) Fab, the fragment which contains a monovalent antigen-binding fragment of an antibody molecule, which can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)2 is a dimer of two Fab′ fragments held together by two disulfide bonds;
4) Fv, a genetically engineered fragment containing the variable region of the light chain and the variable region of the heavy chain expressed as two chains; and
(5) Single chain antibody (SCA or scFv), a genetically engineered molecule containing the variable region of the light chain and the variable region of the heavy chain, linked by a suitable polypeptide linker as a genetically fused single chain molecule.
(6) scFv-Fc, is created by fusing single-chain Fv (scFv) with a hinge region from an immunoglobulin (Ig), such as an IgG and Fc regions.

In one embodiment, the antibody provided herein is a monoclonal antibody. In another embodiment, the antigen-binding fragment provided herein is a single chain Fv (scFv), a diabody, a tandem scFv, a scFv-Fc bivalent molecule, an Fab, Fab′, Fv, or F(ab′)2.

In one embodiment, the term “bivalent molecule” or “BV” refers to a molecule capable of binding to two separate targets at the same time. The bivalent molecule is not limited to having two and only two binding domains and can be a polyvalent molecule or a molecule comprised of linked monovalent molecules. The binding domains of the bivalent molecule can selectively recognize the same epitope or different epitopes located on the same target or located on a target that originates from different species. The binding domains can be linked in any of a number of ways, including, but not limited to, disulfide bonds, peptide bridging, amide bonds, and other natural or synthetic linkages known in the art (Spatola et al., “Chemistry and Biochemistry of Amino Acids, Peptides and Proteins,” B. Weinstein, eds., Marcel Dekker, New York, p. 267 (1983) (general review); Morley, J. S., “Trends Pharm Sci” (1980) pp. 463-468 (general review); Hudson et al., Int J Pept Prot Res (1979) 14, 177-185; Spatola et al., Life Sci (1986) 38, 1243-1249; Hann, M. M., J Chem Soc Perkin Trans I (1982) 307-314; Almquist et al., J Med Chem (1980) 23, 1392-1398; Jennings-White et al., Tetrahedron Lett (1982) 23, 2533; Szelke et al., European Application EP 45665; Chemical Abstracts 97, 39405 (1982); Holladay, et al., Tetrahedron Lett (1983) 24, 4401-4404; and Hruby, V. J., Life Sci (1982) 31, 189-199).

Sequence alignment methods that can be used to achieve the desired sequence alignment include but are not solely restricted to pair-wise alignment methods or multiple-sequence alignment methods, as will be understood by a skilled artisan. Sequence alignments can be stored in a wide variety of text-based file formats. In one embodiment, this is achieved by converting in some embodiments, any format, for example, a FASTA or GenBank, SwissProt, Entrez, and EMBL format, using conversion programs and programming packages, such as READSEQ, EMBOSS, and BioPerl, BioRuby. It is to be understood that a skilled artisan can convert, modify, score, update and/or store the sequences as necessary using any program or storage media, as will be appreciated by the skilled artisan.

In some embodiments, the term “sequence alignment” includes use of any program or method, as understood by a skilled artisan, that is used to perform nucleic acid or amino acid sequence alignments to yield results that can be readily probed, assessed, and subjected to mathematical and statistical calculations. In one embodiment, methods for sequence or structure alignment are well known in the art, and include alignments based on sequence and structural homology, as will be understood by a skilled artisan.

In one embodiment, the term “homology,” “homolog” or “homologous” refer to sequence identity, or to structural identity, or functional identity. By using the term “homology” and the other like forms, it is to be understood that any molecule, whether nucleic acid or peptide, that functions similarly, and/or contains sequence identity, and/or is conserved structurally so that it approximates the reference sequence, is to be considered as part of this invention. In another embodiment, the terms “homology,” “homolog,” or “homologous,” in any instance, indicate that the sequence referred to, whether an amino acid sequence or a nucleic acid sequence, exhibits at least 86% correspondence with the indicated sequence. In another embodiment, the amino acid sequence or nucleic acid sequence exhibits at least 90% correspondence with the indicated sequence. In another embodiment, the amino acid sequence or nucleic acid sequence exhibits at least 92% correspondence with the indicated sequence. In another embodiment, the amino acid sequence or nucleic acid sequence exhibits at least 95% correspondence with the indicated sequence. In another embodiment, the amino acid sequence or nucleic acid sequence exhibits at least 95% or more correspondence with the indicated sequence. In another embodiment, the amino acid sequence or nucleic acid sequence exhibits at least 97% or more correspondence with the indicated sequence. In another embodiment, the amino acid sequence or nucleic acid sequence exhibits 97%-100% correspondence to the indicated sequence. In another embodiment, the amino acid sequence or nucleic acid sequence exhibits 100% correspondence to the indicated sequence. Similarly, in one embodiment, the reference to a correspondence to a particular sequence includes both direct correspondence, as well as homology to that sequence as herein defined.

Accordingly, in one embodiment, the term “non-homologous” refers to an amino acid sequence or nucleic acid sequence that exhibits no more than 85% correspondence with the indicated sequence. In another embodiment, the amino acid sequence or nucleic acid sequence exhibits no more than 75% correspondence with the indicated sequence. In another embodiment, the amino acid sequence or nucleic acid sequence exhibits no more than 65-74% correspondence with the indicated sequence. In another embodiment, the amino acid sequence or nucleic acid sequence exhibits no more than 55-64% correspondence with the indicated sequence. In another embodiment, the amino acid sequence or nucleic acid sequence exhibits no more than 45-54% correspondence with the indicated sequence. In another embodiment, the amino acid sequence or nucleic acid sequence exhibits no more than 35-44% correspondence with the indicated sequence. In another embodiment, the amino acid sequence or nucleic acid sequence exhibits no more than 35-44% correspondence with the indicated sequence. In another embodiment, the amino acid sequence or nucleic acid sequence exhibits no more than 15-34% correspondence with the indicated sequence. In another embodiment, the amino acid sequence or nucleic acid sequence exhibits no more than 5-14% correspondence with the indicated sequence. In another embodiment, the amino acid sequence or nucleic acid sequence exhibits no more than 0.1-4% correspondence with the indicated sequence. In another embodiment, the term “non-homologous can be used interchangeably with the term “low sequence similarity.”

In one embodiment, the light chain contains CDR1, CDR2 and CDR3 sequences that are listed above or have homology more than 70%, for example, more than 75%, 80%, 85%, 90%, 91%, 92%, 93%, 94%, 95%, 96%, 97%, 98%, or 99%; for example with 10, 9, 8, 7, 6, 5, 4, 3, 2, or 1 mutation(s), deletion or insertion of amino acids.

In one embodiment, the light chain contains FR1, FR2 and FR3 sequences that are listed above or have homology more than 70%, for example, more than 75%, 80%, 85%, 90%, 91%, 92%, 93%, 94%, 95%, 96%, 97%, 98%, or 99%; for example with 10, 9, 8, 7, 6, 5, 4, 3, 2, or 1 mutation(s), deletion or insertion of amino acids.

In one embodiment, the heavy chain contains CDR1, CDR2 and CDR3 sequences that are listed above or have homology more than 70%, for example, more than 75%, 80%, 85%, 90%, 91%, 92%, 93%, 94%, 95%, 96%, 97%, 98%, or 99%; for example with 10, 9, 8, 7, 6, 5, 4, 3, 2, or 1 mutation(s), deletion or insertion of amino acids.

In one embodiment, the heavy chain contains FR1, FR2 and FR3 sequences that are listed above or have homology more than 70%, for example, more than 75%, 80%, 85%, 90%, 91%, 92%, 93%, 94%, 95%, 96%, 97%, 98%, or 99%; for example with 10, 9, 8, 7, 6, 5, 4, 3, 2, or 1 mutation(s), deletion or insertion of amino acids.

In one embodiment, a full antibody is one of the types of IgG1, IgG2, IgG3 or IgG4. In another embodiment, Mut-gy1 scFv was engineered into an IgG1 full antibody.

In one embodiment, a full antibody has a constant region of lambda, kappa or a mutated one from them. In another embodiment, Mut-gy1 scFv was engineered to a full antibody using a CL2 constant region.

In one embodiment, the heavy chain comprises a constant region of SEQ ID NO: 59, or a sequence having homology of more than 70%, for example, more than 75%, 80%, 85%, 90%, 91%, 92%, 93%, 94%, 95%, 96%, 97%, 98%, or 99%; for example with 10, 9, 8, 7, 6, 5, 4, 3, 2, or 1 mutation(s), deletion or insertion of amino acids.

In one embodiment, the light chain comprises a constant region of SEQ ID NO: 67, or a sequence having homology of more than 70%, for example, more than 75%, 80%, 85%, 90%, 91%, 92%, 93%, 94%, 95%, 96%, 97%, 98%, or 99%; for example with 10, 9, 8, 7, 6, 5, 4, 3, 2, or 1 mutation(s), deletion or insertion of amino acids.

In one embodiment, there may or may not be linker peptide(s) between signal peptide and variable region, between variable region and constant region of heavy and/or light chain. One example of such linker peptides is encoded by restriction enzyme sites.

In one embodiment, the antibody or antigen-binding fragment thereof is displayed on yeast cell surface. In another embodiment, the antibody or antigen-binding fragment thereof is coated on nanoparticle surface. In another embodiment, the antibody or antigen-binding fragment thereof is displayed on mammalian cell surface, such as T cells, NK cells or other human or other mammalian cells. In yet another embodiment, the antibody or antigen-binding fragment thereof is produced as secretory protein by yeast, E. coli or mammalian cells.

In one embodiment, the term “binds,” “binding,” or grammatical equivalents, refers to the compositions having affinity for each other. “Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In some embodiments, the antibody or antigen-binding fragment thereof has modifications. The modification is one as further defined herein below. In some embodiments, the modification is a N-terminus modification. In another embodiment, the modification is a C-terminal modification. In another embodiment, the modification is in the middle of the protein. In one embodiment, the secretable form of the antibody or antigen-binding fragment comprises an N-terminal modification that allows binding to an Immunoglobulin (Ig) hinge region. In another embodiment, the Ig hinge region is from but is not limited to, an immunoglobulin hinge region. In some embodiments, the modification is a direct modification on antibody or antigen-binding fragment thereof. In other embodiments, the modification is indirect modification bridged by one or more other peptides, proteins, chemicals, carbohydrate or even secondary antibodies.

Additional post-translational modifications encompassed by the invention include, for example, e.g., N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of procaryotic host cell expression.

In one embodiment, the term “polypeptide” generally refers to the antibody, antigen-binding fragments, or variants of the present invention.

In one embodiment, the polypeptide of this invention comprises an amino acid substitution. In one embodiment, the amino acid substitution is conservative. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In another embodiment, the amino acid substitution is not a conservative one that results in enhanced activity of the mutated polypeptide compared to the native polypeptide.

The antibodies or antigen-binding fragments of this invention can be produced by any synthetic or recombinant process, such as those well known in the art. The antibodies or antigen-binding fragments of the invention can further be modified to alter biophysical or biological properties by means of techniques known in the art. For example, the polypeptide can be modified to increase its stability against proteases, or to modify its lipophilicity, solubility, or binding affinity to its native receptor.

In some embodiments, antibody fragments may be prepared by proteolytic hydrolysis of the antibody or by expression in E. coli or mammalian cells (e.g., Chinese hamster ovary cell culture or other protein expression systems) of DNA encoding the fragment. Antibody fragments can, in some embodiments, be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

A “variant” of a polypeptide, antibody, or protein of the present invention, in one embodiment, refers to an amino acid sequence that is altered with respect to the referenced polypeptide, antibody, or protein by one or more amino acids. In the present invention, a variant of a polypeptide retains the antibody-binding property of the referenced protein. In another embodiment, a “variant” refers to the antigen-binding fragment of the present invention. In yet another embodiment, the variant is a variant of the antigen-binding fragment that retains specificity for a target or marker. The variant may have “conservative” changes, wherein a substituted amino acid has similar structural or chemical properties (e.g., replacement of leucine with isoleucine). In another embodiment, the variants have conservative amino acid substitutions at one or more predicted non-essential amino acid residues. In another embodiment, a “conservative amino acid substitution” is one in which the amino acid residue is replaced with an amino acid residue having a side chain with a similar charge, where, in other embodiments, the opposite is the case for “non-conservative substitutions.” Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein may routinely be expressed, and the functional and/or biological activity of the encoded protein can be determined using techniques described herein or by routinely modifying techniques known in the art. Analogous minor variations may also include amino acid deletions or insertions, or both. Guidance in determining which amino acid residues may be substituted, inserted, or deleted without abolishing immunological reactivity may be found using computer programs well known in the art, for example, DNASTAR software.

In one embodiment, the term “framework region” or “FR” are those variable domain residues other than the hypervariable region residues. The framework regions have been precisely defined. See, e.g., Kabat, E. A. et al., Sequences of Proteins of Immunological Interest, US Dept. Health and Human Services, National Institutes of Health, USA (5th ed. 1991). Each variable domain typically has four FRs identified as FR1, FR2, FR3, and FR4. In some embodiments, “FR” also refers to an antibody variable region comprising amino acid residues abutting or proximal to, but outside of the CDR regions, i.e., regions which directly interact with the antigen, acting as the recognition element of the antibody molecule within the variable region of an antibody. In one embodiment, the term “framework region” is intended to mean each domain of the framework that is separated by the CDRs. In some embodiments, the sequences of the framework regions of different light or heavy chains are relatively conserved within a species. The combined heavy and light chain framework regions of an antibody serve to position and align the CDRs for proper binding to the antigen.

In one embodiment, the term “CDR” or “complementarity determining region” refers to amino acid residues comprising non-contiguous antigen combining sites found within the variable region of both heavy and light chain polypeptides. In other embodiments, the term “CDR” will comprise regions as described by Kabat et al., J. Biol. Chem. 252, 6609-6616 (1977) and Kabat et al., Sequences of protein of immunological interest. (1991), and Chothia and Lesk, J. Mol. Biol. 196:901-917 (1987) and MacCallum et al., J. Mol. Biol. 262:732-745 (1996). The amino acids of the CDRs of the variable domains were initially defined by Kabat, based on sequence variability, to consist of amino acid residues 31-35B (H1), 50-65 (H2), and 95-102 (H3) in the human heavy chain variable domain (VH) and amino acid residues 24-34 (L1), 50-56 (L2), and 89-97 (L3) in the human light chain variable domain (VL), using Kabat's numbering system for amino acid residues of an antibody. See Kabat et al., sequences of proteins of immunological interest, US Dept. Health and Human Services, NIH, USA (5th ed. 1991). Chothia and Lesk, J. Mol. Biol. 196:901-917 (1987) presented another definition of the CDRs based on residues that included in the three-dimensional structural loops of the variable domain regions, which were found to be important in antigen binding activity. Chothia et al. defined the CDRs as consisting of amino acid residues 26-32 (H1), 52-56 (H2), and 95-102 (H3) in the human heavy chain variable domain (VH), and amino acid residues 24-34 (L 1), 50-56 (L2), and 89-97 (L3) in the human light chain variable domain (VL). Combining the CDR definitions of Kabat and Chothia, the CDRs consist of amino acid residues 26-35B (H1), 50-65 (H2), and 95-102 (H3) in human VH and amino acid residues 24-34 (L1), 50-56 (L2), and 89-97 (L3) in human VL, based on Kabat's numbering system.

In some embodiments, a “variable region,” when used in reference to an antibody or a heavy or light chain thereof, is intended to mean the amino terminal portion of an antibody which confers antigen binding onto the molecule and which is not the constant region. The term is intended to include functional fragments thereof, which maintain some of all of the binding function of the whole variable region. Therefore, the term “heteromeric variable region binding fragments” is intended to mean at least one heavy chain variable region and at least one light chain variable region or functional fragment thereof assembled into a heteromeric complex. Heteromeric variable region binding fragments include, for example, functional fragments such as Fab, F(ab)2, Fv, single chain Fv (scfv), and the like. Such functional fragments are well known to those skilled in the art. Accordingly, the use of these terms in describing functional fragments of a heteromeric variable region is intended to correspond to the definitions well known to those skilled in the art. Such terms are described in, for example, Harlow and Lane, Antibodies: A Laboratory Manual, Cold Spring Harbor Laboratory, New York (1989); Molec. Biology and Biotechnology: A Comprehensive Desk Reference (Myers, R. A. (ed.), New York: VCH Publisher, Inc.); Huston et al., Cell Biophysics, 22:189-224 (1993); PlGckthun and Skerra, Meth. Enzymol., 178:497-515 (1989) and in Day, E. D., Advanced Immunochemistry, Second Ed., Wiley-Liss, Inc., New York, N.Y. (1990).

In one embodiment, the polypeptide of this invention is an isoform of the isolated polypeptide. In one embodiment, “isoform” refers to a version of a molecule, for example, a protein or polypeptide of the present invention, with only slight differences to another isoform of the same protein or polypeptide. In one embodiment, isoforms are produced from different but related genes, or in another embodiment, they arise from the same gene by alternative splicing. In another embodiment, isoforms are caused by single nucleic acid polymorphisms.

In one embodiment, the isolated polypeptide of this invention is a fragment of the native protein. In one embodiment, “fragment” refers to a protein or polypeptide that is shorter or comprises fewer amino acids than the full length protein or polypeptide. In another embodiment, fragment refers to a nucleic acid that is shorter or comprises fewer nucleic acids than the full length nucleic acid. In another embodiment, the fragment is an N-terminal fragment. In another embodiment, the fragment is a C-terminal fragment. In one embodiment, the fragment of this invention is an intrasequential section of the protein, peptide, or nucleic acid. In another embodiment, the fragment is a functional intrasequential section of the protein, peptide or nucleic acid. In another embodiment, the fragment is a functional intrasequential section within the protein, peptide or nucleic acid. In another embodiment, the fragment is an N-terminal functional fragment. In one embodiment, the fragment is a C-terminal functional fragment.

In one embodiment, the term “functional fragment” refers to a fragment that maintains a certain degree of biological activity as compared to the wild type despite it being a modified version of the native or wild type antibody or polypeptide. This degree of activity could range from moderate to high as compared to the wild type, where the “activity” refers to its natural biophysical or biochemical characteristics, e.g., binding ability, affinity, half-life, etc.

In one embodiment, an isolated polypeptide of this invention comprises a derivate of a polypeptide of this invention. “Derivative” is to be understood as referring, in some embodiments, to less than the full-length portion of the native sequence of the protein in question. In some embodiments, a “derivative” may further comprise (at its termini and/or within the sequence itself) non-native sequences, i.e., sequences which do not form part of the native protein in question. The term “derivative” also includes within its scope molecular species produced by conjugating chemical groups to the amino residue side chains of the native proteins or fragments thereof, wherein the chemical groups do not form part of the naturally-occurring amino acid residues present in the native proteins.

Methods of making antibodies and antibody fragments are known in the art. (See, for example, Harlow and Lane, Antibodies: A Laboratory Manual, Cold Spring Harbor Laboratory, New York, 1988, incorporated herein by reference).

Antibodies can be produced by the immunization of various animals, including mice, rats, rabbits, goats, primates, humans, and chickens, with a target antigen such as PSMA or peptide fragments of PSMA containing the anti-PSMA epitope of the present invention. In one embodiment, the antibody or antigen-binding fragment is purified prior to immunization of the animal. In one embodiment, the antibody or antigen-binding fragment of the present invention can be purified by methods known in the art, for example, gel filtration, ion exchange, affinity chromatography, etc. Affinity chromatography or any of a number of other techniques known in the art can be used to isolate polyclonal or monoclonal antibodies from serum, ascites fluid, or hybridoma supernatants.

“Purified” means that the monoclonal antibody is separated from at least some of the proteins normally associated with the monoclonal antibody and preferably separated from all cellular materials other than proteins.

Also provided are chemically modified derivatives of antibodies of the invention which may provide additional advantages such as increased solubility, stability, in vivo or in vitro circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol, and the like. The antibodies may be modified at random positions within the molecule, or at predetermined positions within the molecule and may include one, two, three or more attached chemical moieties.

The polymer may be of any molecular weight, and may be branched or unbranched. For polyethylene glycol, the molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2560, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleic acids 18:2745-2750 (1999); and Caliceti et al., Bioconjug. Chem. 10:638-646 (1999), the disclosures of each of which are incorporated herein by reference.

The polyethylene glycol molecules (or other chemical moieties) should be attached to the antibody with consideration of effects on functional or antigenic domains of the antibody. There are a number of attachment methods available to those skilled in the art, e.g., EP 0 401 384, herein incorporated by reference (coupling PEG to G-CSF); see also Malik et al., Exp. Hematol. 20:1028-1035 (1992) (reporting pegylation of GM-CSF using tresyl chloride). For example, polyethylene glycol may be covalently bound through amino acid residues via a reactive group, such as a free amino or carboxyl group. Reactive groups are those to which an activated polyethylene glycol molecule may be bound. The amino acid residues having a free amino group may include, for example, lysine residues and the N-terminal amino acid residues; those having a free carboxyl group may include aspartic acid residues, glutamic acid residues, and the C-terminal amino acid residue. Sulfhydryl groups may also be used as a reactive group for attaching the polyethylene glycol molecules. in some embodiments, attachment is at an amino group, such as attachment at the N-terminus or lysine group.

As suggested above, polyethylene glycol may be attached to proteins, e.g., antibodies, via linkage to any of a number of amino acid residues. For example, polyethylene glycol can be linked to a protein via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

### Glycosylation Variants

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites are created or removed.

For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Glycosylation of the constant region on N297 may be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyltransferase, such that antibodies expressed in such a cell line exhibit hypofucosylation. PCT Publication WO 03/035835 by Presta describes a variant Chinese Hamster Ovary cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyltransferases (e.g., beta(1,4)-N-acetylglucosaminyltransferase III (GnTIII)) such that antibodies expressed in the engineered cell lines exhibit increased bisecting GlcNac structures which result in increased ADCC activity of the antibodies (see also Umana et al. (1999) Nat. Biotech. 17: 176-180).

Fc Region Variants The variable regions of the antibody described herein can be linked (e.g., covalently linked or fused) to an Fc, e.g., an IgG1, IgG2, IgG3 or IgG4 Fc, which may be of any allotype or isoallotype, e.g., for IgG1: Glm, Glm1(a), Glm2(x), Glm3(f), Glm17(z); for IgG2: G2m, G2m23(n); for IgG3: G3m, G3m21(g1), G3m28(g5), G3m11(b0), G3m5(b1), G3ml3(b3), G3m14(b4), G3m10(b5), G3ml5(s), G3m16(t), G3m6(c3), G3m24(c5), G3m26(u), G3m27(v); and for K: Km, Km1, Km2, Km3 (see, e.g., Jefferies et al. (2009) mAbs 1: 1). In some embodiments, the antibodies variable regions described herein are linked to an Fc that binds to one or more activating Fc receptors (FcγI, Fcγlla, or FcγIIIa), and thereby stimulate ADCC and may cause T cell depletion. In some embodiments, the antibody variable regions described herein are linked to an Fc that causes depletion.

In some embodiments, the antibody variable regions described herein may be linked to an Fc comprising one or more modifications, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, to alter one or more functional properties of the antibody.

The numbering of residues in the Fc region is that of the EU index of Kabat.

The Fc region encompasses domains derived from the constant region of an immunoglobulin, preferably a human immunoglobulin, including a fragment, analog, variant, mutant, or derivative of the constant region. Suitable immunoglobulins include IgG1, IgG2, IgG3, IgG4, and other classes such as IgA, IgD, IgE, and IgM. The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination. In some embodiments, an antibody of this invention has an Fc region other than that of a wild type IgA1. The antibody can have an Fc region from that of IgG (e.g., IgG1, IgG2, IgG3, and IgG4) or other classes such as IgA2, IgD, IgE, and IgM. The Fc can be a mutant form of IgA1.

The constant region of an immunoglobulin is responsible for many important antibody functions, including Fc receptor (FcR) binding and complement fixation. There are five major classes of heavy chain constant region, classified as IgA, IgG, IgD, IgE, IgM, each with characteristic effector functions designated by isotype. For example, IgG is separated into four subclasses known as IgG1, IgG2, IgG3, and IgG4.

Ig molecules interact with multiple classes of cellular receptors. For example, IgG molecules interact with three classes of Fcγ receptors (FcγR) specific for the IgG class of antibody, namely FcγRI, FcγRII, and FcγRIIL. The important sequences for the binding of IgG to the FcγR receptors have been reported to be located in the CH2 and CH3 domains. The serum half-life of an antibody is influenced by the ability of that antibody to bind to an FcR.

In some embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, one may make modifications in the Fc region in order to generate an Fc variant that (a) has increased or decreased ADCC, (b) increased or decreased CDC, (c) has increased or decreased affinity for Clq and/or (d) has increased or decreased affinity for an Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc., substitutions therein, e.g., of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli, such as proline iminopeptidase. In other embodiments, one or more glycosylation sites within the Fec domain may be removed. Residues that are typically glycosylated (e.g., asparagine) may confer cytolytic response. Such residues may be deleted or substituted with unglycosylated residues (e.g., alanine). In other embodiments, sites involved in interaction with complement, such as the Clq binding site, may be removed from the Fc region. For example, one may delete or substitute the EKK sequence of human IgG1. In some embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgG1. Specific examples of variant Fc domains are disclosed, for example, in WO 97/34631 and WO 96/32478.

In one embodiment, the hinge region of Fc is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320, and 322 can be replaced with a different amino acid residue such that the antibody has an altered affinity for an effector ligand but retains the antigen-binding ability of the parent antibody. The effector ligand to which affinity is altered can be, for example, an Fc receptor or the CI component of complement. This approach is described in further detail in U.S. Pat. Nos. 5,624,821 and 5,648,260, both by Winter et al.

In another example, one or more amino acids selected from amino acid residues 329, 331, and 322 can be replaced with a different amino acid residue such that the antibody has altered Clq binding and/or reduced or abolished CDC. This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region may be modified to increase ADCC and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in abat (WO00/42072).

Other Fc modifications that can be made to Fes are those for reducing or ablating binding to FcγR and/or complement proteins, thereby reducing or ablating Fc-mediated effector functions such as ADCC, antibody-dependent cellular phagocytosis (ADCP), and CDC. Exemplary modifications include but are not limited to substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcγR and complement interactions include substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; WO00/42072; WO01/58957; WO02/06919; WO04/016750; WO04/029207; WO04/035752; WO04/074455; WO04/099249; WO04/063351; WO05/070963; WO05/040217, WO05/092925 and WO06/020114).

Fc variants that enhance affinity for an inhibitory receptor FcγRIIb may also be used. Such variants may provide an Fc fusion protein with immune-modulatory activities related to FcγRIIb cells, including, for example, B cells and monocytes. In one embodiment, the Fc variants provide selectively enhanced affinity to FcγRIIb relative to one or more activating receptors. Modifications for altering binding to FcγRIIb include one or more modifications at a position selected from 234, 235, 236, 237, 239, 266, 267, 268, 325, 326, 327, 328, and 332, according to the EU index. Exemplary substitutions for enhancing FcγRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical or immunological based assays) known in the art, including but not limited to, equilibrium methods (e.g., ELISA, or radioimmunoassay), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods, including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In some embodiments, the antibody is modified to increase its biological half-life. Various approaches are possible. For example, this may be done by increasing the binding affinity of the Fc region for FcRn. For example, one or more of the following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fe region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 434H, 434F, 434Y, and 434M. Other variants that increase Fc binding to FcRn include: 250E, 250Q, 428L, 428F, 250Q/428L (Hinton et al, 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In some embodiments, hybrid IgG isotypes with particular biological characteristics may be used. For example, an IgG1/IgG3 hybrid variant may be constructed by substituting IgG 1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 4221, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Thus a hybrid variant IgG antibody may be constructed chat comprises one or more substitutions, e.g., one or more of the following amino acid substitutions: 233E, 234L, 235L, 236G (referring to an insertion of a glycine at position 236), and 321H.

Moreover, the binding sites on human IgG1 for FcγRI, FcγRII, FcγRIII, and FcRn have been mapped, and variants with improved binding have been described (see Shields, R. L. et al. (2001) J. Biol. Chem. 276:6591-6604). Specific mutations at positions 256, 290, 298, 333, 334, and 339 were shown to improve binding to FcγRIII. Additionally, the following combination mutants were shown to improve FcγRIII binding: T256A/S298A, S298A/E333A, S298A/K224A, and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/1332E and S239D/1332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L, and P396L mutations, which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include: S298A/E333A/L334A, S239D/1332E, S239D/1332E/A330L, L235V/F243L/R292P/Y300L/P396L, and M428L/N434S.

In some embodiments, an Fc is chosen that has reduced binding to FcγRs. An exemplary Fc, e.g., IgG1 Fc, with reduced FcγR binding, comprises the following three amino acid substitutions: L234A, L235E, and G237A.

In some embodiments, an Fc is chosen that has reduced complement fixation. An exemplary Fc, e.g., IgG1 Fc, with reduced complement fixation, has the following two amino acid substitutions: A330S and P331S.

In some embodiments, an Fc is chosen that has essentially no effector function, i.e., it has reduced binding to FcγRs and reduced complement fixation. An exemplary Fc, e.g., IgG1 Fc, that is effectorless, comprises the following five mutations: L234A, L235E, G237A, A330S, and P331S.

When using an IgG4 constant domain, it is usually preferable to include the substitution S228P, which mimics the hinge sequence in IgG1 and thereby stabilizes IgG4 molecules.

Fc Mutations that Increase Serum Half-Life

In some embodiments, the Fc region or Fc domain of the anti-HIV gp120 directed antibody comprises amino acid modifications that promote an increased serum half-life of the antigen-binding molecule. Mutations that increase the half-life of an antibody have been described. In one embodiment, the Fc region or Fc domain of one or both of the CD3-targeting heavy chain and the HIV antigen-targeting heavy chain comprise a methionine to tyrosine substitution at position 252 (EU numbering), a serine to threonine substitution at position 254 (EU numbering), and a threonine to glutamic acid substitution at position 256 (EU numbering). See, e.g., U.S. Pat. No. 7,658,921. This type of mutant, designated as a “YTE mutant” exhibits a four-fold increased half-life relative to wild-type versions of the same antibody (DalFAcqua, et al., J Biol Chem, 281: 23514-24 (2006); Robbie, et aL., Antimicrob Agents Chemotherap., 57(12):6147-6153 (2013)). In certain embodiments, the Fc region or Fc domain of one or both of the CD3-targeting heavy chain and the HIV antigen-targeting heavy chain comprise an IgG constant domain comprising one, two, three, or more amino acid substitutions of amino acid residues at positions 251-257, 285-290, 308-314, 385-389, and 428-436 (EU numbering). Alternatively, M428L and N434S (“LS”) substitutions can increase the pharmacokinetic half-life of the multi-specific antigen-binding molecule. In other embodiments, the Fc region or Fec domain of one or both of the CD3-targeting heavy chain and the HIV antigen-targeting heavy chain comprise a M428L and N434S substitution (EU numbering). In other embodiments, the Fc region or Fc domain of one or both of the CD3-targeting heavy chain and the HIV antigen-targeting heavy chain comprise T250Q and M428L (EU numbering) mutations. In other embodiments, the Fc region or Fc domain of one or both of the CD3-targeting heavy chain and the HIV antigen-targeting heavy chain comprise H433K and N434F (EU numbering) mutations.

Fc Mutations that Enhance Effector Activity

In some embodiments, the Fc region or Fc domain of the anti-HIV gpl20 directed antibody comprises post-translational and/or amino acid modifications that increase effector activity, e.g., have improved FcγIIIa binding and increased antibody-dependent cellular cytotoxicity (ADCC). In some embodiments, the Fc region or Fc domain of the anti-HIV gpl20 directed antibody comprises DE modifications (i.e., S239D and 1332E by EU numbering) in the Fc region. In some embodiments, the Fc region or Fc domain of the anti-HIV gpl20 directed antibody comprises DEL modifications (i.e., S239D, 1332E, and A330L by EU numbering) in the Fc region. In some embodiments, the Fc region or Fc domain of the anti-HIV gpl20 directed antibody comprises DEA modifications (i.e., S239D, 1332E, and G236A by EU numbering) in the Fc region. In some embodiments, the Fc region or Fec domain of the anti-HIV gp120 directed antibody comprises DEAL modifications (i.e., S239D, 1332E, G236A, and A330L by EU numbering) in the Fc region. See, e.g., U.S. Pat. Nos. 7,317,091; 7,662,925; 8,039,592; 8,093,357; 8,093,359; 8,383,109; 8,388,955; 8,735,545; 8,858,937; 8,937,158; 9,040,041; 9,353,187; 10,184,000; and 10,584,176. Additional amino acid modifications that increase effector activity, e.g., have improved Fcyllla binding and increased antibody-dependent cellular cytotoxicity (ADCC), include without limitation (EU numbering) F243L/R292P/Y300L/V305I/P396L; S298A/E333A/K334A; or L234Y/L235Q/G236W/S239M/H268D/D270E/S298A on a first Fc domain and D270E/K326D/A330M/K334E on a second Fc domain. Amino acid mutations that increase Clq binding and complement-dependent cytotoxicity (CDC) include without limitation (EU numbering) S267E/H268F/S324T or K326W/E333 S. Fc region mutations that enhance effector activity are reviewed in, e.g., Wang, et al., Protein Cell (2018) 9(1): 63-73; and Saunders, Front Immunol. (2019) 10: 1296. In other embodiments, the anti-HIV gp120 directed antibody or antigen-binding fragment thereof has modified glycosylation, which, e.g., may be introduced post-translationally or through genetic engineering. In some embodiments, the anti-HIV gp120 directed antibody or antigen-binding fragment thereof is afucosylated, e.g., at a glycosylation site present in the antibody or antigen-binding fragment thereof. Most approved monoclonal antibodies are of the IgG1 isotype, where two N-linked biantennary complex-type oligosaccharides are bound to the Fc region. The Fc region exercises the effector function of ADCC through its interaction with leukocyte receptors of the FcγR family. Afucosylated monoclonal antibodies are monoclonal antibodies engineered so that the oligosaccharides in the Fc region of the antibody do not have any fucose sugar units.

### Multivalent Antibodies

In one embodiment, the antibodies of this disclosure may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0130105. In each case, at least one of the binding sites will comprise an epitope, motif, or domain associated with a DLL3 isoform.

In one embodiment, the antibodies are bispecific antibodies in which the two chains have different specificities, as described in Millstein et al., 1983, Nature, 305:537-539. Other embodiments include antibodies with additional specificities, such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

As stated above, multivalent antibodies may immunospecifically bind to different epitopes of the desired target molecule or may immunospecifically bind to both the target molecule as well as a heterologous epitope, such as a heterologous polypeptide or solid support material. In some embodiments, the multivalent antibodies may include bispecific antibodies or trispecific antibodies. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980) and for treatment of HIV infection (WO 91/00360, WO 92/200373, and EP 03089). Heteroconjugate antibodies may be made using any convenient cross-linking methods. Suitable cross-linking agents are well known in the art and are disclosed in U.S. Pat. No. 4,676,980, along with a number of cross-linking techniques.

In some embodiments, antibody variable domains with the desired binding specificities (antibody-antigen combining sites) are fused to immunoglobulin constant domain sequences, such as an immunoglobulin heavy chain constant domain comprising at least part of the hinge, CH2, and/or CH3 regions, using methods well known to those of ordinary skill in the art.

In some embodiments, the isolated anti-HIV antibody is a bispecific antibody comprising a first antigen-binding arm binding to a first antigen and a second antigen-binding arm binding to a second antigen, wherein the first antigen and the second antigen are different, and wherein the first antigen-binding arm comprising the heavy chain amino acid sequence and the light chain amino acid sequence as specified above.

In some embodiments, the second antigen is selected from CD3, FcγRI (CD64), FcγRII (CD32), FcγRIII (CD 16); CD89, CCR5, CD4, CD8, CD28, CD 137, CTLA-4, gp41, killer cell immunoglobulin like receptor, three Ig domains and long cytoplasic tail 1 (KIR3DL1), killer cell immunoglobulin like receptor, three Ig domains and long cytoplasmic tail 1 (KIR3DL1), killer cell immunoglobulin like receptor, two Ig domains and long cytoplasmic tail 1 (KIR2DL1), killer cell immunoglobulin like receptor, two Ig domains and long cytoplasmic tail 2 (KIR2DL2), killer cell immunoglobulin like receptor, two Ig domains and long cytoplasmic tail 3 (KIR2DL3), killer cell lectin like receptor C1 (KLRC1), killer cell lectin like receptor C2 (KLRC2), killer cell lectin like receptor C3 (KLRC3), killer cell lectin like receptor C4 (KLRC4), killer cell lectin like receptor D1 (KLRD1), killer cell lectin like receptor K1 (KLRK1), natural cytotoxicity triggering receptor 3 (NCR3 or NKp30), natural cytotoxicity triggering receptor 2 (NCR2 or NK-p44), natural cytotoxicity triggering receptor 1 (NCR1 or NK-p46), CD226 (DNAM-1), cytotoxic and regulatory T cell molecule (CRTAM or CD355), signaling lymphocytic activation molecule family member 1 (SLAMF1), CD48 (SLAMF2), lymphocyte antigen 9 (LY9 or SLAMF3), CD244 (2B4 or SLAMF4), CD84 (SLAMF5), SLAM family member 6 (SLAMF6 or NTB-A), SLAM family member 7 (SLAMF7 or CRACC), CD27 (TNFRSF7), semaphorin 4D (SEMA4D or CD 100), CD 160 (NK1), an immune checkpoint protein (e.g., PD-1, PD-L1, CTLA-4), and a second epitope of gpl20.

### Antibody Derivatives

An antibody provided herein may be further modified to contain additional nonproteinaceous moieties that are known in the art and readily available. The moieties suitable for derivatization of the antibody include but are not limited to water-soluble polymers.

Non-limiting examples of water-soluble polymers include, but are not limited to, PEG, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Another modification of the antibodies described herein is pegylation. An antibody can be pegylated to, for example, increase the biological (e.g., serum) half-life of the antibody. To pegylate an antibody, the antibody, or fragment thereof, typically is reacted with PEG, such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI—CIO) alkoxy- or aryloxy-polyethylene glycol or polyethylene glycol-maleimide. In some embodiments, the antibody to be pegylated is an aglycosylated antibody. Methods for pegylating proteins are known in the art and can be applied to the antibodies described herein. See, for example, EP 0 154 316 by Nishimura et al. and EP0401384 by Ishikawa et al.

This disclosure also encompasses a human monoclonal antibody described herein conjugated to a therapeutic agent, a polymer, a detectable label, or enzyme. In one embodiment, the therapeutic agent is a cytotoxic agent. In one embodiment, the polymer is PEG.

### Nucleic Acids

In one embodiment, this disclosure also provides polynucleic acids comprising, or alternatively consisting of, a nucleic acid sequence encoding a polypeptide chain of the disclosed antibody or antigen-binding fragment thereof or variant thereof. This disclosure also encompasses polynucleic acids that hybridize under high stringency, or alternatively, under intermediate or lower stringency hybridization conditions, e.g., as defined supra, to polynucleic acids complementary to nucleic acids having a polynucleic acid sequence that encodes a polypeptide chain of the disclosed antibody or antigen-binding fragment thereof or variant thereof.

In another embodiment, the polynucleic acids are obtained, and the nucleic acid sequence of the polynucleic acids can be determined by any method known in the art. Alternatively, a polynucleic acid encoding an antibody (including molecules comprising, or alternatively consisting of, antibody fragments or variants thereof) are generated from nucleic acid from a suitable source. If a clone containing a nucleic acid encoding a particular antibody is not available, but the sequence of the antibody molecule is known, a nucleic acid encoding the immunoglobulin may be chemically synthesized in native or optimized codons for specific species or obtained from a suitable source (e.g., an antibody cDNA library, or a cDNA library generated from, or nucleic acid, preferably poly A+RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleic acid probe specific for the particular gene sequence to identify, e.g., a cDNA clone from a cDNA library that encodes the antibody. Amplified nucleic acids generated by PCR may then be cloned into replicable cloning vectors using any method well known in the art.

In some embodiments, the term “nucleic acid” refers to polynucleic acid or to oligonucleic acids such as deoxyribonucleic acid (DNA) and, where appropriate, ribonucleic acid (RNA) or mimetic thereof. The term should also be understood to include, as equivalents, analogs of either RNA or DNA made from nucleic acid analogs and, as applicable to the embodiment being described, single (sense or antisense) and double-stranded polynucleic acids. This term includes oligonucleic acids composed of naturally-occurring nucleobases, sugars, and covalent internucleoside (backbone) linkages, as well as oligonucleic acids having non-naturally-occurring portions, which function similarly. Such modified or substituted oligonucleic acids are often preferred over native forms because of desirable properties such as, for example, enhanced cellular uptake, enhanced affinity for a nucleic acid target, and increased stability in the presence of nucleases.

As will be appreciated by one skilled in the art, a fragment or derivative of a nucleic acid sequence or gene that encodes for a protein or peptide can still function in the same manner as the entire, wild type gene or sequence. Likewise, forms of nucleic acid sequences can have variations with respect to wild type sequences, nevertheless encoding a protein or peptide, or fragments thereof, retaining wild type function exhibiting the same biological effect, despite these variations. Each of these represents a separate embodiment of the present invention.

The nucleic acids can be produced by any synthetic or recombinant process, such as those well known in the art. Nucleic acids can further be modified to alter biophysical or biological properties by means of techniques known in the art. For example, the nucleic acid can be modified to increase its stability against nucleases (e.g., “end-capping”), or to increase expression level by codon-optimization, or to modify its lipophilicity, solubility, or binding affinity to complementary sequences.

Methods for modifying nucleic acids to achieve specific purposes are disclosed in the art, for example, in Sambrook et al. (1989). Moreover, the nucleic acid sequences of the invention can include one or more portions of nucleic acid sequence that are non-coding for the protein of interest. The invention further provides DNA sequences which encode proteins similar to those encoded by sequences as described herein, but which differ in terms of their codon sequence due to the degeneracy of the genetic code or allelic variations (naturally-occurring base changes in the species population which may or may not result in an amino acid change), which may encode the proteins of the invention described herein, as well. Variations in the DNA sequences, which are caused by point mutations or by induced modifications (including insertion, deletion, and substitution) to enhance the activity, half-life or production of the polypeptides encoded thereby, are also encompassed in the invention.

DNA encoding the antibodies or antigen-binding fragments provided herein is readily isolated and sequenced using conventional procedures (e.g., by using oligonucleic acid probes that are capable of binding specifically to genes encoding the heavy and light chains of the antibodies). Once isolated, the DNA may be placed into expression vectors, which are then transfected into host cells such as E. coli cells, simian COS cells, Chinese hamster ovary (CHO) cells, yeast cells or myeloma cells that do not otherwise produce immunoglobulin protein, to obtain the synthesis of the antibodies in the recombinant host cells. Recombinant production of antibodies is described in more detail below.

In one embodiment, the nucleic acid molecule encoding an scFv Mut-gy1 comprising the amino acid sequence of SEQ ID NO: 3. In one embodiment, the nucleic acid molecule comprises the nucleotide sequence of SEQ ID NO: 2.

In one embodiment, the nucleic acid molecule comprises a nucleic acid sequence encoding a light chain comprising the amino acid sequence of SEQ ID NO: 5. For example, in one embodiment, the nucleic acid sequence encoding a light chain comprises the nucleotide sequence of SEQ ID NO: 4. In one embodiment, the nucleic acid molecule comprises a nucleic acid sequence encoding a VL FR1 comprising the amino acid sequence of SEQ ID NO: 7. For example, in one embodiment, the nucleic acid sequence encoding a VL FR1 comprises the nucleotide sequence of SEQ ID NO: 6. In one embodiment, the nucleic acid molecule comprises a nucleic acid sequence encoding a VL CDR1 comprising the amino acid sequence of SEQ ID NO: 9. For example, in one embodiment, the nucleic acid sequence encoding a VL CDR1 comprises the nucleotide sequence of SEQ ID NO: 8. In one embodiment, the nucleic acid molecule comprises a nucleic acid sequence encoding a VL FR2 comprising the amino acid sequence of SEQ ID NO: 11. For example, in one embodiment, the nucleic acid sequence encoding a VL FR2 comprises the nucleotide sequence of SEQ ID NO: 10. In one embodiment, the nucleic acid molecule comprises a nucleic acid sequence encoding a VL CDR2 comprising the amino acid sequence of SEQ ID NO: 13. For example, in one embodiment, the nucleic acid sequence encoding a VL CDR2 comprises the nucleotide sequence of SEQ ID NO: 12. In one embodiment, the nucleic acid molecule comprises a nucleic acid sequence encoding a VL FR3 comprises an amino acid sequence of SEQ ID NO: 15. For example, in one embodiment, the nucleic acid sequence encoding a VL FR3 comprises the nucleotide sequence of SEQ ID NO: 14. In one embodiment, the nucleic acid molecule comprises a nucleic acid sequence encoding a VL CDR3 comprises the amino acid sequence of SEQ ID NO: 17. For example, in one embodiment, the nucleic acid sequence encoding a VL CDR3 comprises the nucleotide sequence of SEQ ID NO: 16. In one embodiment, the nucleic acid molecule comprises a nucleic acid sequence encoding a VL FR4 comprising the amino acid sequence of SEQ ID NO: 19. For example, in one embodiment, the nucleic acid sequence encoding a VL FR4 comprises the nucleotide sequence of SEQ ID NO: 18.

In one embodiment, the nucleic acid molecule comprises a nucleic acid sequence encoding a heavy chain comprising the amino acid sequence of SEQ ID NO: 21. For example, in one embodiment, the nucleic acid sequence encoding a heavy chain comprises the nucleotide sequence of SEQ ID NO: 20. In one embodiment, the nucleic acid molecule comprises a nucleic acid sequence encoding a VH FR1 comprising the amino acid sequence of SEQ ID NO: 23. For example, in one embodiment, the nucleic acid sequence encoding a VH FR1 comprises the nucleotide sequence of SEQ ID NO: 22. In one embodiment, the nucleic acid molecule comprises a nucleic acid sequence encoding a VH CDR1 comprising the amino acid sequence of SEQ ID NO: 25. For example, in one embodiment, the nucleic acid sequence encoding a VH CDR1 comprises the nucleotide sequence of SEQ ID NO: 24. In one embodiment, the nucleic acid molecule comprises a nucleic acid sequence encoding a VH FR2 comprising the amino acid sequence of SEQ ID NO: 27. For example, in one embodiment, the nucleic acid sequence encoding a VH FR2 comprises the nucleotide sequence of SEQ ID NO: 26. In one embodiment, the nucleic acid molecule comprises a nucleic acid sequence encoding a VH CDR2 comprising the amino acid sequence of SEQ ID NO: 29. For example, in one embodiment, the nucleic acid sequence encoding a VH CDR2 comprises the nucleotide sequence of SEQ ID NO: 28. In one embodiment, the nucleic acid molecule comprises a nucleic acid sequence encoding a VH FR3 comprising the amino acid sequence of SEQ ID NO: 31. For example, in one embodiment, the nucleic acid sequence encoding a VH FR3 comprises the nucleotide sequence of SEQ ID NO: 30. In one embodiment, the nucleic acid molecule comprises a nucleic acid sequence encoding a VH CDR3 comprising the amino acid sequence of SEQ ID NO: 33. For example, in one embodiment, the nucleic acid sequence encoding a VH CDR3 comprises the nucleotide sequence of SEQ ID NO: 32. In one embodiment, the nucleic acid molecule comprises a nucleic acid sequence encoding a VH FR4 comprising the amino acid sequence of SEQ ID NO: 35. For example, in one embodiment, the nucleic acid sequence encoding a VH FR4 comprises the nucleotide sequence of SEQ ID NO: 34. In one embodiment, the nucleic acid molecule comprises a nucleic acid sequence encoding a scFv linker comprising the amino acid sequence of SEQ ID NO: 37. For example, in one embodiment, the nucleic acid sequence encoding a scFv linker comprises the nucleotide sequence of SEQ ID NO: 36.

For example, in one embodiment, the nucleic acid molecule encodes Mut-gy1. In one embodiment, the nucleic acid molecule encodes Mut-gy1, comprising the amino acid sequence of SEQ ID NO: 3. For example, in one embodiment, the nucleic acid sequence encoding a Mut-gy1 comprises the nucleotide sequence of SEQ ID NO: 2. In one embodiment, the nucleic acid molecule encoding Mut-gy1 comprises a nucleotide sequence encoding a light chain comprising the amino acid sequence of SEQ ID NO: 5 and a nucleotide sequence encoding a heavy chain comprising the amino acid sequence of SEQ ID NO: 21. For example, in one embodiment, the nucleotide sequence encoding a light chain comprises the nucleotide sequence of SEQ ID NO: 4, and the nucleotide sequence encoding a heavy chain comprises the nucleotide sequence of SEQ ID NO: 20.

In one embodiment, the nucleic acid molecule encoding Mut-gy1 comprises a nucleotide sequence encoding a VL FR1 comprising the amino acid sequence of SEQ ID NO: 7; a nucleotide sequence encoding a VL CDR1 comprising the amino acid sequence of SEQ ID NO: 9; a nucleotide sequence encoding a VL FR2 comprising the amino acid sequence of SEQ ID NO: 11; a nucleotide sequence encoding a VL CDR2 comprising the amino acid sequence of SEQ ID NO: 13; a nucleotide sequence encoding a VL FR3 comprising the amino acid sequence of SEQ ID NO: 15; a nucleotide sequence encoding a VL CDR3 comprising the amino acid sequence of SEQ ID NO: 17; a nucleotide sequence encoding a VL FR4 comprising the amino acid sequence of SEQ ID NO: 19; a nucleotide sequence encoding a VH FR1 comprising the amino acid sequence of SEQ ID NO: 23; a nucleotide sequence encoding a VH CDR1 comprising the amino acid sequence of SEQ ID NO: 25; a nucleotide sequence encoding a VH FR2 comprising the amino acid sequence of SEQ ID NO: 27; a nucleotide sequence encoding a VH CDR2 comprising the amino acid sequence of SEQ ID NO: 29; a nucleotide sequence encoding a VH FR3 comprising the amino acid sequence of SEQ ID NO: 31; a nucleotide sequence encoding a VH CDR3 comprising the amino acid sequence of SEQ ID NO: 33; a nucleotide sequence encoding a VH FR4 comprising the amino acid sequence of SEQ ID NO: 35; and/or a nucleotide sequence encoding a scFv linker comprising the amino acid sequence of SEQ ID NO: 37.

For example, in one embodiment, the nucleotide sequence encoding a VL FR1 comprises the nucleotide sequence of SEQ ID NO: 6; the nucleotide sequence encoding a VL CDR1 comprises the nucleotide sequence of SEQ ID NO: 8; the nucleotide sequence encoding a VL FR2 comprises the nucleotide sequence of SEQ ID NO: 10; the nucleotide sequence encoding a VL CDR2 comprises the nucleotide sequence of SEQ ID NO: 12; the nucleotide sequence encoding a VL FR3 comprises the nucleotide sequence of SEQ ID NO: 14; the nucleotide sequence encoding a VL CDR3 comprises the nucleotide sequence of SEQ ID NO: 16; the nucleotide sequence encoding a VL FR4 comprises the nucleotide sequence of SEQ ID NO: 18; the nucleotide sequence encoding a VH FR1 comprises the nucleotide sequence of SEQ ID NO: 22; the nucleotide sequence encoding a VH CDR1 comprises the nucleotide sequence of SEQ ID NO: 24; the nucleotide sequence encoding a VH FR2 comprises the nucleotide sequence of SEQ ID NO: 26; the nucleotide sequence encoding a VH CDR2 comprises the nucleotide sequence of SEQ ID NO: 28; the nucleotide sequence encoding a VH FR3 comprises the nucleotide sequence of SEQ ID NO: 30; the nucleotide sequence encoding a VH CDR3 comprises the nucleotide sequence of SEQ ID NO: 32; the nucleotide sequence encoding a VH FR4 comprises the nucleotide sequence of SEQ ID NO: 34; and/or the nucleotide sequence encoding a scFv linker comprises the nucleotide sequence of SEQ ID NO: 36.

In one embodiment, the nucleic acid molecule encodes an antibody or antigen-binding fragment thereof comprising one or more mutations. For example, in one embodiment, the nucleic acid molecule comprises a nucleotide sequence encoding a VL FR2 comprising the amino acid sequence of SEQ ID NO: 39, where SEQ ID NO: 39 comprises a V→A point mutation with respect to SEQ ID NO: 11. For example, in one embodiment, the nucleotide sequence encoding the mutant VL FR2 comprises the nucleotide sequence of SEQ ID NO: 38. In one embodiment, the nucleic acid molecule comprises a nucleotide sequence encoding a VL CDR2 comprising the amino acid sequence of SEQ ID NO: 41, where SEQ ID NO: 41 comprises a G→E point mutation with respect to SEQ ID NO: 13. For example, in one embodiment, the nucleotide sequence encoding the mutant VL CDR2 comprises the nucleotide sequence of SEQ ID NO: 40. In one embodiment, the nucleic acid molecule comprises a nucleotide sequence encoding a VL FR4 comprising the amino acid sequence of SEQ ID NO: 43, where SEQ ID NO: 43 comprises a V→A point mutation with respect to SEQ ID NO: 19. For example, in one embodiment, the nucleotide sequence encoding the mutant VL FR4 comprises the nucleotide sequence of SEQ ID NO: 42. In one embodiment, the nucleic acid molecule comprises a nucleotide sequence encoding a VH CDR1 comprising the amino acid sequence of SEQ ID NO: 45, where SEQ ID NO: 45 comprises a S→F point mutation with respect to SEQ ID NO: 25. For example, in one embodiment, the nucleotide sequence encoding the mutant VH CDR1 comprises the nucleotide sequence of SEQ ID NO: 44. In one embodiment, the nucleic acid molecule comprises a nucleotide sequence encoding a VH FR3 comprising the amino acid sequence of SEQ ID NO: 47, where SEQ ID NO: 47 comprises an I→V point mutation with respect to SEQ ID NO: 31. For example, in one embodiment, the nucleotide sequence encoding the mutant VH FR3 comprises the nucleotide sequence of SEQ ID NO: 46. In one embodiment, the nucleic acid molecule comprises a nucleotide sequence encoding a VH CDR3 comprising the amino acid sequence of SEQ ID NO: 49, where SEQ ID NO: 49 comprises a D→G point mutation with respect to SEQ ID NO: 33. For example, in one embodiment, the nucleotide sequence encoding the mutant VH CDR3 comprises the nucleotide sequence of SEQ ID NO: 48. In one embodiment, the nucleic acid molecule comprises a nucleotide sequence encoding a VH FR4 comprising the amino acid sequence of SEQ ID NO: 51, where SEQ ID NO: 51 comprises a G→E point mutation with respect to SEQ ID NO: 35. For example, in one embodiment, the nucleotide sequence encoding the mutant VH FR4 comprises the nucleotide sequence of SEQ ID NO: 50.

In one embodiment, the nucleic acid molecule encodes an antibody fragment comprising a scFv denoted herein as Mut-gy1-st. In one embodiment, the nucleic acid molecule encoding Mut-gy1-st comprises a nucleotide sequence encoding a VL FR2 comprising the amino acid sequence of SEQ ID NO: 39; a nucleotide sequence encoding a VH CDR1 comprising the amino acid sequence of SEQ ID NO: 45, and a nucleotide sequence encoding a VH FR4 comprising the amino acid sequence of SEQ ID NO: 51. In one embodiment, the nucleic acid molecule encoding Mut-gy1-st comprises a nucleotide sequence encoding a VL FR1 comprising the amino acid sequence of SEQ ID NO: 7; a nucleotide sequence encoding a VL CDR1 comprising the amino acid sequence of SEQ ID NO: 9; a nucleotide sequence encoding a VL FR2 comprising the amino acid sequence of SEQ ID NO: 39; a nucleotide sequence encoding a VL CDR2 comprising the amino acid sequence of SEQ ID NO: 13; a nucleotide sequence encoding a VL FR3 comprising the amino acid sequence of SEQ ID NO: 15; a nucleotide sequence encoding a VL CDR3 comprising the amino acid sequence of SEQ ID NO: 17; a nucleotide sequence encoding a VL FR4 comprising the amino acid sequence of SEQ ID NO: 19; a nucleotide sequence encoding a VH FR1 comprising the amino acid sequence of SEQ ID NO: 23; a nucleotide sequence encoding a VH CDR1 comprising the amino acid sequence of SEQ ID NO: 45; a nucleotide sequence encoding a VH FR2 comprising the amino acid sequence of SEQ ID NO: 27; a nucleotide sequence encoding a VH CDR2 comprising the amino acid sequence of SEQ ID NO: 29; a nucleotide sequence encoding a VH FR3 comprising the amino acid sequence of SEQ ID NO: 31; a nucleotide sequence encoding a VH CDR3 comprising the amino acid sequence of SEQ ID NO: 33; a nucleotide sequence encoding a VH FR4 comprising the amino acid sequence of SEQ ID NO: 51; and/or a nucleotide sequence encoding a scFv linker comprising the amino acid sequence of SEQ ID NO: 37.

In one embodiment, the nucleotide sequence encoding a VL FR1 comprises the nucleotide sequence of SEQ ID NO: 6; the nucleotide sequence encoding a VL CDR1 comprises the nucleotide sequence of SEQ ID NO: 8; the nucleotide sequence encoding a VL FR2 comprises the nucleotide sequence of SEQ ID NO: 38; the nucleotide sequence encoding a VL CDR2 comprises the nucleotide sequence of SEQ ID NO: 12; the nucleotide sequence encoding a VL FR3 comprises the nucleotide sequence of SEQ ID NO: 14; the nucleotide sequence encoding a VL CDR3 comprises the nucleotide sequence of SEQ ID NO: 16; the nucleotide sequence encoding a VL FR4 comprises the nucleotide sequence of SEQ ID NO: 18; the nucleotide sequence encoding a VH FR1 comprises the nucleotide sequence of SEQ ID NO: 22; the nucleotide sequence encoding a VH CDR1 comprises the nucleotide sequence of SEQ ID NO: 44; the nucleotide sequence encoding a VH FR2 comprises the nucleotide sequence of SEQ ID NO: 26; the nucleotide sequence encoding a VH CDR2 comprises the nucleotide sequence of SEQ ID NO: 28; the nucleotide sequence encoding a VH FR3 comprises the nucleotide sequence of SEQ ID NO: 30; the nucleotide sequence encoding a VH CDR3 comprising comprises the nucleotide sequence of SEQ ID NO: 32; the nucleotide sequence encoding a VH FR4 comprises the nucleotide sequence of SEQ ID NO: 50; and/or the nucleotide sequence encoding a scFv linker comprises the nucleotide sequence of SEQ ID NO: 36.

For example, in one embodiment, the composition comprises a nucleic acid molecule encoding an antibody fragment comprising a scFv denoted herein as Mut-gy1-2. In one embodiment, the nucleic acid molecule encoding Mut-gy1-2 comprises a nucleotide sequence encoding VL CDR2 comprising the amino acid sequence of SEQ ID NO: 41; a nucleotide sequence encoding VL FR4 comprising the amino acid sequence of SEQ ID NO: 43; a nucleotide sequence encoding VH CDR1 comprising the amino acid sequence of SEQ ID NO: 45, and/or a nucleotide sequence encoding VH FR3 comprising the amino acid sequence of SEQ ID NO: 47.

In one embodiment, the nucleic acid molecule encoding Mut-gy1-2 comprises a nucleotide sequence encoding a VL FR1 comprising the amino acid sequence of SEQ ID NO: 7; a nucleotide sequence encoding a VL CDR1 comprising the amino acid sequence of SEQ ID NO: 9; a nucleotide sequence encoding a VL FR2 comprising the amino acid sequence of SEQ ID NO: 11; a nucleotide sequence encoding a VL CDR2 comprising the amino acid sequence of SEQ ID NO: 41; a nucleotide sequence encoding a VL FR3 comprising the amino acid sequence of SEQ ID NO: 15; a nucleotide sequence encoding a VL CDR3 comprising the amino acid sequence of SEQ ID NO: 17; a nucleotide sequence encoding a VL FR4 comprising the amino acid sequence of SEQ ID NO: 43; a nucleotide sequence encoding a VH FR1 comprising the amino acid sequence of SEQ ID NO: 23; a nucleotide sequence encoding a VH CDR1 comprising the amino acid sequence of SEQ ID NO: 45; a nucleotide sequence encoding a VH FR2 comprising the amino acid sequence of SEQ ID NO: 27; a nucleotide sequence encoding a VH CDR2 comprising the amino acid sequence of SEQ ID NO: 29; a nucleotide sequence encoding a VH FR3 comprising the amino acid sequence of SEQ ID NO: 47; a nucleotide sequence encoding a VH CDR3 comprising the amino acid sequence of SEQ ID NO: 33; a nucleotide sequence encoding a VH FR4 comprising the amino acid sequence of SEQ ID NO: 35; and/or a nucleotide sequence encoding a scFv linker comprising the amino acid sequence of SEQ ID NO: 37.

In one embodiment, the nucleotide sequence encoding a VL FR1 comprises the nucleotide sequence of SEQ ID NO: 6; the nucleotide sequence encoding a VL CDR1 comprises the nucleotide sequence of SEQ ID NO: 8; the nucleotide sequence encoding a VL FR2 comprises the nucleotide sequence of SEQ ID NO: 10; the nucleotide sequence encoding a VL CDR2 comprises the nucleotide sequence of SEQ ID NO: 40; the nucleotide sequence encoding a VL FR3 comprises the nucleotide sequence of SEQ ID NO: 14; the nucleotide sequence encoding a VL CDR3 comprises the nucleotide sequence of SEQ ID NO: 16; the nucleotide sequence encoding a VL FR4 comprises the nucleotide sequence of SEQ ID NO: 42; the nucleotide sequence encoding a VH FR1 comprises the nucleotide sequence of SEQ ID NO: 22; the nucleotide sequence encoding a VH CDR1 comprises the nucleotide sequence of SEQ ID NO: 44; the nucleotide sequence encoding a VH FR2 comprises the nucleotide sequence of SEQ ID NO: 26; the nucleotide sequence encoding a VH CDR2 comprises the nucleotide sequence of SEQ ID NO: 28; the nucleotide sequence encoding a VH FR3 comprises the nucleotide sequence of SEQ ID NO: 46; the nucleotide sequence encoding a VH CDR3 comprises the nucleotide sequence of SEQ ID NO: 32; the nucleotide sequence encoding a VH FR4 comprises the nucleotide sequence of SEQ ID NO: 34; and/or the nucleotide sequence encoding a scFv linker comprises the nucleotide sequence of SEQ ID NO: 36.

In one embodiment, the nucleic acid molecule encodes an antibody fragment comprising a scFv denoted herein as Mut-gy1-3. In one embodiment, the nucleic acid molecule encoding Mut-gy1-3 comprises a nucleotide sequence encoding a VH CDR1 comprising the amino acid sequence of SEQ ID NO: 45, and a nucleotide sequence encoding a VH CDR3 comprising the amino acid sequence of SEQ ID NO: 49. In one embodiment, Mut-gy1 comprises a nucleotide sequence encoding a VL FR1 comprising the amino acid sequence of SEQ ID NO: 7; a nucleotide sequence encoding a VL CDR1 comprising the amino acid sequence of SEQ ID NO: 9; a nucleotide sequence encoding a VL FR2 comprising the amino acid sequence of SEQ ID NO: 11; a nucleotide sequence encoding a VL CDR2 comprising the amino acid sequence of SEQ ID NO: 13; a nucleotide sequence encoding a VL FR3 comprising the amino acid sequence of SEQ ID NO: 15; a nucleotide sequence encoding a VL CDR3 comprising the amino acid sequence of SEQ ID NO: 17; a nucleotide sequence encoding a VL FR4 comprising the amino acid sequence of SEQ ID NO: 19; a nucleotide sequence encoding a VH FR1 comprising the amino acid sequence of SEQ ID NO: 23; a nucleotide sequence encoding a VH CDR1 comprising the amino acid sequence of SEQ ID NO: 45; a nucleotide sequence encoding a VH FR2 comprising the amino acid sequence of SEQ ID NO: 27; a nucleotide sequence encoding a VH CDR2 comprising the amino acid sequence of SEQ ID NO: 29; a nucleotide sequence encoding a VH FR3 comprising the amino acid sequence of SEQ ID NO: 31; a nucleotide sequence encoding a VH CDR3 comprising the amino acid sequence of SEQ ID NO: 49; and/or a nucleotide sequence encoding a VH FR4 comprising the amino acid sequence of SEQ ID NO: 35. In some embodiments, the nucleic acid molecule comprises a nucleotide sequence encoding a scFv linker comprising the amino acid sequence of SEQ ID NO: 37.

In one embodiment, the nucleotide sequence encoding a VL FR1 comprises the nucleotide sequence of SEQ ID NO: 6; the nucleotide sequence encoding a VL CDR1 comprises the nucleotide sequence of SEQ ID NO: 8; the nucleotide sequence encoding a VL FR2 comprises the nucleotide sequence of SEQ ID NO: 10; the nucleotide sequence encoding a VL CDR2 comprises the nucleotide sequence of SEQ ID NO: 12; the nucleotide sequence encoding a VL FR3 comprises the nucleotide sequence of SEQ ID NO: 14; the nucleotide sequence encoding a VL CDR3 comprises the nucleotide sequence of SEQ ID NO: 16; the nucleotide sequence encoding a VL FR4 comprises the nucleotide sequence of SEQ ID NO: 18; the nucleotide sequence encoding a VH FR1 comprises the nucleotide sequence of SEQ ID NO: 22; the nucleotide sequence encoding a VH CDR1 comprises the nucleotide sequence of SEQ ID NO: 44; the nucleotide sequence encoding a VH FR2 comprises the nucleotide sequence of SEQ ID NO: 26; the nucleotide sequence encoding a VH CDR2 comprises the nucleotide sequence of SEQ ID NO: 28; the nucleotide sequence encoding a VH FR3 comprises the nucleotide sequence of SEQ ID NO: 30; the nucleotide sequence encoding a VH CDR3 comprises the nucleotide sequence of SEQ ID NO: 48; and/or the nucleotide sequence encoding a VH FR4 comprises the nucleotide sequence of SEQ ID NO: 34. In some embodiments, the nucleotide sequence encoding a scFv linker comprises the nucleotide sequence of SEQ ID NO: 36.

In some embodiments, the nucleic acid molecule encodes PSMAb or a polypeptide chain thereof. In one embodiment, the nucleic acid molecule comprises a nucleotide sequence that encodes a heavy chain comprising the amino acid sequence of SEQ ID NO: 68. In one embodiment, the nucleic acid molecule comprises a nucleotide sequence encoding a heavy chain having a signal peptide, wherein the heavy chain having a signal peptide comprises the amino acid sequence of SEQ ID NO: 53. In one embodiment, the nucleic acid molecule comprises a nucleotide sequence encoding a heavy chain signal peptide comprising the amino acid sequence of SEQ ID NO: 55. In one embodiment, the nucleic acid molecule comprises a nucleotide sequence encoding a heavy chain variable region comprising the amino acid sequence of SEQ ID NO: 57. In one embodiment, the nucleic acid molecule comprises a nucleotide sequence encoding a heavy chain constant region of SEQ ID NO: 59. In one embodiment, the nucleic acid molecule comprises a nucleotide sequence encoding a light chain comprising the amino acid sequence of SEQ ID NO: 69. In one embodiment, the nucleic acid molecule comprises a nucleotide sequence encoding a light chain having a signal peptide, wherein the light chain having a signal peptide comprises the amino acid sequence of SEQ ID NO: 61. In one embodiment, the nucleic acid molecule comprises a nucleotide sequence encoding the light chain signal peptide comprising the amino acid sequence of SEQ ID NO: 63. In one embodiment, the nucleic acid molecule comprises a nucleotide sequence encoding a light chain variable region comprising the amino acid sequence of SEQ ID NO: 65. In one embodiment, the nucleic acid molecule comprises a nucleotide sequence encoding a light chain constant region of SEQ ID NO: 67. In one embodiment, the nucleotide sequence encoding a heavy chain having a signal peptide comprises the nucleotide sequence of SEQ ID NO: 52. In one embodiment, the nucleotide sequence encoding heavy chain signal peptide comprises the nucleotide sequence of SEQ ID NO: 54. In one embodiment, the nucleotide sequence encoding a heavy chain variable region comprises the nucleotide sequence of SEQ ID NO: 56. In one embodiment, the nucleotide sequence encoding a heavy chain constant region comprises the nucleotide sequence of SEQ ID NO: 58. In one embodiment, the nucleotide sequence encoding a light chain having a signal peptide comprises the nucleotide sequence of SEQ ID NO: 60. In one embodiment, the nucleotide sequence encoding the light chain signal peptide comprises the nucleotide sequence of SEQ ID NO: 62. In one embodiment, the nucleotide sequence encoding a light chain variable region comprises the nucleotide sequence of SEQ ID NO: 64. In one embodiment, the nucleotide sequence encoding a light chain constant region comprises the nucleotide sequence of SEQ ID NO: 66.

In some embodiments, the nucleic acid molecule comprises a nucleotide sequence having homology to one or more nucleotide sequences described herein. For example, in some embodiments, the nucleic acid molecule comprises a nucleotide sequence having 70% or more, 75% or more, 80% or more, 82% or more, 85% or more, 87% or more, 90% or more, 91% or more, 92% or more, 93% or more, 94% or more, 95% or more, 96% or more, 97% or more, 98% or more, 99% or more, or 99.5% or more homology to a nucleotide sequence described herein.

In another aspect, this disclosure also provides the methods to express or produce the recombinant protein of the anti-PSMA antibody or antigen-binding fragment thereof using various protein expression systems.

In one embodiment, the invention also provides transformed cells and progeny thereof into which a nucleic acid molecule encoding an antibody or antigen-binding fragment, has been introduced by means of recombinant DNA techniques in vitro, ex vivo or in vivo. The transformed cells, eukaryotic or prokaryotic, may be used to produce a recombinant antibody or antigen-binding fragment thereof for purification, or for in situ or secretory expression for various purposes, such as diagnosis or therapy for tumor. The transformed cells can be propagated, the introduced nucleic acid is transcribed, or encoded protein is expressed. It is understood that a progeny cell may not be identical to the parental cell, since there may be mutations that occur during replication. Transformed cells include but are not limited to prokaryotic and eukaryotic cells such as bacteria, fungi, plant, insect, and animal (e.g., mammalian, including human) cells. The cells may be present in culture, in a cell, tissue or organ ex vivo or present in a subject.

Typically cell transformation employs a vector. The term “vector” refers to, e.g., a plasmid, virus, such as a viral vector, or other vehicle known in the art that can be manipulated by insertion or incorporation of a nucleic acid, for genetic manipulation (i.e., “cloning vectors”), or can be used to transcribe or translate the inserted polynucleic acid (i.e., “expression vectors”). Such vectors are useful for introducing nucleic acids, including a nucleic acid that encodes an antibody operably linked with an expression control element, and expressing the encoded protein in vitro (e.g., in solution or in solid phase), in cells or in vivo.

In one embodiment, the expression vector(s) is(are) transferred to a host cell by conventional techniques, and the transfected cells are then cultured by conventional techniques to produce an antibody or antigen-binding fragment of the invention. Thus, the invention includes host cells containing polynucleic acid(s) encoding an antibody of the invention (e.g., whole antibody, a heavy or light chain thereof, or portion thereof, or a single chain antibody, or a fragment or variant thereof), operably linked to a heterologous promoter. In other embodiments, for the expression of entire antibody molecules, vectors encoding both the heavy and light chains are co-expressed in the host cell for expression of the entire immunoglobulin molecule.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleic acid coding sequences, express an antibody molecule of the invention in situ. These include, but are not limited to, bacteriophage particles engineered to express antibody fragments or variants thereof (single chain antibodies), microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3, NS0 cells) harboring recombinant expression constructs containing promoters derived from the genome of mammalian cells (e.g., metallothionein promoter) or from mammalian viruses (e.g., the adenovirus late promoter; the vaccinia virus 7.5K promoter; CMV promoter or EF1α promoter). Preferably, bacterial cells such as Escherichia coli, and more preferably, eukaryotic cells, especially for the expression of a whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (11990); Bebbington et al., Bio/Techniques 10:169 (1992); Keen and Hale, Cytotechnology 18:207 (1996)). These references are incorporated in their entireties by reference herein.

A vector used to transform a cell or a host-expression vector generally contains at least an origin of replication for propagation in the cell. Control elements, including expression control elements as set forth herein, present within a vector, are included to facilitate transcription and translation. The term “expression control element” is intended to include, at a minimum, one or more components whose presence can influence expression, and can include components other than or in addition to promoters or enhancers, for example, leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of a gene of interest, stop codons, etc.

Vectors can include a selection marker. As is known in the art, “selection marker” means a gene that allows for the selection of cells containing the gene. “Positive selection” refers to a process whereby only cells that contain the selection marker will survive upon exposure to the positive selection. Drug resistance is one example of a positive selection marker; cells containing the marker will survive in culture medium containing the selection drug, and cells, which do not contain the marker will die. Such markers include drug resistance genes such as neo, which confers resistance to G418, hygr, which confers resistance to hygromycin, or puro, which confers resistance to puromycin, among others. Other positive selection marker genes include genes that allow identification or screening of cells containing the marker. These genes include genes for fluorescent proteins (GFP), the lacZ gene, the alkaline phosphatase gene, and surface markers such as CD8, among others.

Vectors can contain negative selection markers. “Negative selection” refers to a process whereby cells containing a negative selection marker are killed upon exposure to an appropriate negative selection agent. For example, cells which contain the herpes simplex virus-thymidine kinase (HSV-tk) gene (Wigler et al., Cell 11:223 (1977)) are sensitive to the drug gancyclovir (GANC). Similarly, the gpt gene renders cells sensitive to 6-thioxanthine.

Mammalian expression systems further include vectors specifically designed for in vivo and ex vivo expression. Such systems include adeno-associated virus (AAV) vectors (U.S. Pat. No. 5,604,090). AAV vectors have previously been shown to provide expression of Factor IX in humans and in mice at levels sufficient for therapeutic benefit (Kay et al., Nat. Genet. 24:257 (2000); Nakai et al., Blood 91:4600 (1998)). Adenoviral vectors (U.S. Pat. Nos. 5,700,470, 5,731,172, and 5,928,944), herpes simplex virus vectors (U.S. Pat. No. 5,501,979), and retroviral (e.g., lentivirus vectors are useful for infecting dividing as well as non-dividing cells and foamy virus) vectors (U.S. Pat. Nos. 5,624,820, 5,693,508, 5,665,577, 6,013,516 and 5,674,703 and WIPO publications WO92/05266 and WO92/14829) and papilloma virus vectors (e.g., human and bovine papilloma virus) have all been employed in gene therapy (U.S. Pat. No. 5,719,054). Vectors also include cytomegalovirus (CMV) based vectors (U.S. Pat. No. 5,561,063). Vectors that efficiently deliver genes to cells of the intestinal tract have been developed and also may be used (see, e.g., U.S. Pat. Nos. 5,821,235, 5,786,340, and 6,110,456). In yeast, vectors that facilitate integration of foreign nucleic acid sequences into a chromosome, via homologous recombination, for example, are known in the art and can be used. Yeast artificial chromosomes (YAC) are typically used when the inserted nucleic acids are too large for more conventional vectors (e.g., greater than about 12 kb).

In one embodiment, phagemid vectors for use in the invention include any available in the art suitable for the production of the antibodies/antibody templates/FR libraries of the present invention and include phagemid vectors pCB04, p1T1, pIT2, CANTAB 6, pComb 3 HS. Filamentous vectors and methods of phagemid construction are described in, for example, U.S. Pat. Nos. 6,054,312 and 6,803,230, each incorporated herein by reference. Bacteriophage display systems involving non-filamentous bacteriophage vectors known as cytoplasmic bacteriophage or lytic phage can also be utilized as described in for example, U.S. Pat. No. 5,766,905, incorporated herein by reference.

Suitable bacterial expression constructs for use with the present invention include, but are not limited to the pCAL, pUC, pET, pETBlue™ (Novagen), pBAD, pLEX, pTrcHis2, pSE280, pSE380, pSE420 (Invitrogen), pKK223-2 (Clontech), pTrc99A, pKK223-3, pRIT2T, pMC1871, pEZZ 18 (Pharmacia), pBluescript II SK (Stratagene), pALTER-Ex1, pALTER-Ex2, pGEMEX (Promega), pFivE (MBI), pQE (Qiagen) commercially available expression constructs, and their derivatives, and others known in the art. In some embodiments, the construct may also include, a virus, a plasmid, a bacmid, a phagemid, a cosmid, or a bacteriophage.

The use of liposomes for introducing various compositions into cells, including nucleic acids, is known to those skilled in the art (see, e.g., U.S. Pat. Nos. 4,844,904, 5,000,959, 4,863,740, and 4,975,282). A carrier comprising a natural polymer, or a derivative or a hydrolysate of a natural polymer, described in WO 94/20078 and U.S. Pat. No. 6,096,291, is suitable for mucosal delivery of molecules, such as polypeptides and polynucleic acids. Piperazine based amphilic cationic lipids useful for gene therapy also are known (see, e.g., U.S. Pat. No. 5,861,397). Cationic lipid systems also are known (see, e.g., U.S. Pat. No. 5,459,127). Accordingly, viral and non-viral vector means of delivery into cells or tissue, in vitro, in vivo, and ex vivo are included.

In one embodiment, nucleic acid sequences can be “operably linked,” i.e., positioned, to ensure the functioning of an expression control sequence. These expression constructs are typically replicable in the cells either as episomes or as integral parts of the cell's chromosomal DNA, and may contain appropriate origins of replication for the respective prokaryotic strain employed for expression. Commonly, expression constructs contain selection markers, such as tetracycline resistance, ampicillin resistance, kanamycin resistance or chlormaphenicol resistance, facilitating detection and/or selection of those bacterial cells transformed with the desired nucleic acid sequences (see, e.g., U.S. Pat. No. 4,704,362). These markers, however, are not exclusionary, and numerous others may be employed, as known to those skilled in the art. In some embodiments, expression constructs contain both positive and negative selection markers.

Similarly, reporter genes may be incorporated within expression constructs to facilitate identification of transcribed products. Accordingly, in one embodiment of the present invention, reporter genes utilized are selected from the group consisting of β-galactosidase, chloramphenicol acetyl transferase, luciferase, and a fluorescent protein.

Prokaryotic promoter sequences regulate expression of the encoded polynucleic acid sequences, and in some embodiments of the present invention, are operably linked to polynucleic acids encoding the polypeptides of this invention. In additional embodiments of the present invention, these promoters are either constitutive or inducible, and provide a means of high and low levels of expression of the polypeptides of this invention, and in some embodiments, for regulated expression of multiple polypeptides of the invention, which in some embodiments are expressed as a fusion protein.

Many well-known bacterial promoters, including the T7 promoter system, the lactose promoter system, typtophan (Trp) promoter system, Trc/Tac Promoter Systems, beta-lactamase promoter system, tetA Promoter systems, arabinose regulated promoter system, Phage T5 Promoter, or a promoter system from phage lambda, may be employed, and others, as well, and comprise embodiments of the present invention. The promoters will typically control expression, optionally with an operator sequence, and may include ribosome binding site sequences, for example, for initiating and completing transcription and translation. According to additional embodiments, the vector may also contain expression control sequences, enhancers that may regulate the transcriptional activity of the promoter, appropriate restriction sites to facilitate cloning of inserts adjacent to the promoter and other necessary information processing sites, such as RNA splice sites, polyadenylation sites and transcription termination sequences as well as any other sequence which may facilitate the expression of the inserted nucleic acid.

In another embodiment, the present invention comprises methods of use of a polynucleic acid, vector, antibodies and/or fragment thereof as herein described and/or compositions comprising the same in treating, inhibiting or preventing.

### PSMA-Targeted Antibody Drug Conjugate

In one embodiment, the invention provides antibody-drug conjugates (ADCs), comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In one specific embodiment, the drugs include, but are not limited to, tubulin inhibitors and DNA cleavage reagents, such as Maytansinoids, Auristatins, Dolastatins, and Calicheamicin. In another embodiment, the invention further provides methods of using the ADCs. In one aspect, an ADC comprises any of the above PSMA antibodies or antibody fragments covalently attached to a cytotoxic agent or a detectable agent.

The use of antibody-drug conjugates for the local delivery of cytotoxic or cytostatic agents, i.e., drugs to kill or inhibit tumor cells in the treatment of cancer (Syrigos and Epenetos (1999) Anticancer Research 19:605-614; Niculescu-Duvaz and Springer (1997) Adv. Drg Del. Rev. 26:151-172; U.S. Pat. No. 4,975,278) allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies '84: Biological And Clinical applications, A. Pinchera et al. (ed.s), pp. 475-506). Maximal efficacy with minimal toxicity is sought thereby. Both polyclonal antibodies and monoclonal antibodies have been reported as useful in these strategies (Rowland et al., (1986) Cancer Immunol. Immunother., 21:183-87). Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al. (2000) Jour. of the Nat. Cancer Inst. 92(19):1573-1581; Mandler et al. (2000) Bioorganic & Med. Chem. Letters 10:1025-1028; Mandler et al. (2002) Bioconjugate Chem. 13:786-791), maytansinoids (EP 1391213; Liu et al., (1996) Proc. Natl. Acad. Sci. USA 93:8618-8623), and calicheamicin (Lode et al. (1998) Cancer Res. 58:2928; Hinman et al. (1993) Cancer Res. 53:3336-3342). The toxins may affect their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Examples of antibody-drug conjugates include ZEVALIN® (ibritumomab tiuxetan, Biogen/Idec), which is an antibody-radioisotope conjugate composed of a murine IgG1 kappa monoclonal antibody directed against the CD20 antigen found on the surface of normal and malignant B lymphocytes and 11 In or 90Y radioisotope bound by a thiourea linker-chelator (Wiseman et al. (2000) Eur. Jour. Nucl. Med. 27(7):766-77; Wiseman et al. (2002) Blood 99(12):4336-42; Witzig et al (2002) J. Clin. Oncol. 20(10):2453-63; Witzig et al. (2002) J. Clin. Oncol. 20(15):3262-69).

Additionally, MYLOTARG™ (gemtuzumab ozogamicin, Wyeth Pharmaceuticals), an antibody drug conjugate composed of a human CD33 antibody linked to calicheamicin, was approved in 2000 for the treatment of acute myeloid leukemia by injection (Drugs of the Future (2000) 25(7):686; U.S. Pat. Nos. 4,970,198; 5,079,233; 5,585,089; 5,606,040; 5,693,762; 5,739,116; 5,767,285; 5,773,001).

Finally, the auristatin peptides, such as monomethyl auristatin E (MMAE), synthetic analogs of dolastatin, were conjugated to chimeric monoclonal antibodies cBR96 (specific to Lewis Y on carcinomas) and cAC10 (specific to CD30 on hematological malignancies) (Doronina et al. (2003) Nature Biotechnology 21(7):778-784). The cAC10 is under therapeutic development.

Further, chemotherapeutic agents useful in the generation of ADCs are described herein. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. See, e.g., WO 93/21232 published Oct. 28, 1993. A variety of radionuclides are available for the production of radioconjugated antibodies. Examples include 212Bi, 1311, 1311n, 90Y, and 186Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987) Science, 238:1098. Carbon-14-labeled 1-isothiocyanatobenzyl-3-methyldiethylene triaminepentaacetic acid (MX-DTPA) is an exemplary chelating agent for conjugation of radionucleic acid to the antibody (WO94/11026).

Conjugates of an antibody and one or more small molecule toxins, such as a calicheamicin, maytansinoids, dolastatins, auristatins, a trichothecene, and CC1065, and the derivatives of these toxins that have toxin activity, are also contemplated herein.

### Maytansinoids:

Maytansine compounds suitable for use as maytansinoid drug moieties are well known in the art, and can be isolated from natural sources according to known methods, produced using genetic engineering techniques (see Yu et al. (2002) PNAS 99:7968-7973), or maytansinol and maytansinol analogs prepared synthetically according to known methods.

Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides). and those having modifications at other positions Exemplary maytansinoid drug moieties also include those having modifications such as: C-9-SH (U.S. Pat. No. 4,424,219) (prepared by the reaction of maytansinol with H2S or P2S5); C-14-alkoxymethyl(demethoxy/CH2OR)(U.S. Pat. No. 4,331,598); C-14-hydroxymethyl or acyloxymethyl (CH2OH or CH2OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

ADCs containing maytansinoids, methods of making same, and their therapeutic use are disclosed, for example, in U.S. Pat. Nos. 5,208,020; 5,416,064; 6,441,163 and European Patent EP 0 425 235 B1, the disclosures of which are hereby expressly incorporated by reference. Liu et al., Proc. Natl. Acad. Sci. USA 93:8618-8623 (1996) described ADCs comprising a maytansinoid designated DM1 linked to the monoclonal antibody C242 directed against human colorectal cancer. The conjugate was found to be highly cytotoxic to cultured colon cancer cells, and showed antitumor activity in an in vivo tumor growth assay. Chari et al., Cancer Research 52:127-131 (1992) describe ADCs in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×10⁵ HER-2 surface antigens per cell. The drug conjugate achieved a degree of cytotoxicity similar to the free maytansinoid drug, which could be increased by increasing the number of maytansinoid molecules per antibody molecule. The A7-maytansinoid conjugate showed low systemic cytotoxicity in mice.

An exemplary maytansinoids embodiment is DM1 (wherein the wavy line indicates the covalent attachment to a linker (L) of an antibody-drug conjugate).

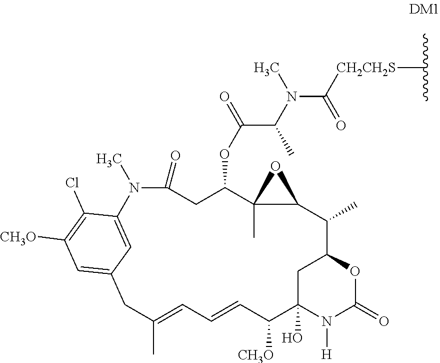

### Auristatins and Dolastatins:

In some embodiments, the ADC comprises an antibody of the invention conjugated to dolastatins or dolostatin peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division (Woyke et al. (2001) Antimicrob. Agents and Chemother. 45(12):3580-3584) and have anticancer (U.S. Pat. No. 5,663,149) and antifungal activity (Pettit et al. (1998) Antimicrob. Agents Chemother. 42:2961-2965). The dolastatin or auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF, disclosed in “Senter et al., Proceedings of the American Association for Cancer Research, Volume 45, Abstract Number 623, presented Mar. 28, 2004 and described in United States Patent Publication No. 2005/0238649, the disclosure of which is expressly incorporated by reference in its entirety.

ADC using MMAE and MMAF with various linker components have been disclosed (US 2005/0238649, U.S. Ser. No. 08/968,742).

An exemplary auristatin embodiment is MMAE (wherein the wavy line indicates the covalent attachment to a linker (L) of an antibody-drug conjugate).

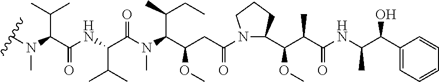 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Another exemplary auristatin embodiment is MMAF, wherein the wavy line indicates the covalent attachment to a linker (L) of an antibody-drug conjugate (US 2005/0238649):

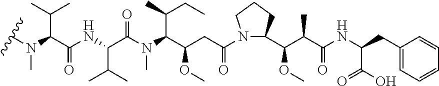 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

Typically, peptide-based drug moieties can be prepared by forming a peptide bond between two or more amino acids and/or peptide fragments. Such peptide bonds can be prepared, for example, according to the liquid phase synthesis method (see E. Schroder and K. Lübke, “The Peptides,” volume 1, pp 76-136, 1965, Academic Press) that is well known in the field of peptide chemistry. The auristatin/dolastatin drug moieties may be prepared according to the methods of: U.S. Pat. Nos. 5,635,483; 5,780,588; Pettit et al. (1989) J. Am. Chem. Soc. 111:5463-5465; Pettit et al. (1998) Anti-Cancer Drug Design 13:243-277; Pettit, G. R., et al. Synthesis, 1996, 719-725; Pettit et al. (1996) J. Chem. Soc. Perkin Trans. 1 5:859-863; and Doronina (2003) Nat Biotechnol 21(7):778-784.

### Calicheamicin:

In other embodiments, the ADC comprises an antibody of the invention conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, and 5,877,296 (all to American Cyanamid Company). Structural analogs of calicheamicin which may be used include, but are not limited to, γ1 I, α2 I, α3 I, N-acetyl-γ1 I, PSAG, and θ1 I (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody-mediated internalization greatly enhances their cytotoxic effects.

### Other Cytotoxic Agents:

Other antitumor agents that can be conjugated to the antibodies of the invention include BCNU, streptozoicin, vincristine, and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and tricothecenes. See, for example, WO 93/21232 published Oct. 28, 1993.

The present invention further contemplates an ADC formed between an antibody and a compound with nucleolytic activity (e.g., a ribonuclease or a DNA endonuclease such as a deoxyribonuclease; DNase).

For selective destruction of the tumor, the antibody may comprise a highly radioactive atom. A variety of radioactive isotopes are available for the production of radioconjugated antibodies. Examples include At211, 1131, 1125, Y90, Re186, Re188, Sm153, Bi212, P32, Pb212, and radioactive isotopes of Lu. When the conjugate is used for detection, it may comprise a radioactive atom for scintigraphic studies, for example, tc99m or 1123, or a spin label for nuclear magnetic resonance (NMR) imaging (also known as magnetic resonance imaging (MRI)), such as iodine-123 again, iodine-131, indium-111, fluorine-19, carbon-13, nitrogen-15, oxygen-17, gadolinium, manganese or iron.

The radio- or other labels may be incorporated into the conjugate in known ways. For example, the peptide may be biosynthesized or may be synthesized by chemical amino acid synthesis using suitable amino acid precursors involving, for example, fluorine-19 in place of hydrogen. Labels such as tc99m or 1123, Re186, Re188, and In111 can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

### PSMA-Targeted Antibody-Drug Conjugate Compounds:

The present invention provides, inter alia, antibody-drug conjugate compounds for targeted delivery of drugs. The inventors have made the discovery that the antibody-drug conjugate compounds have potent cytotoxic and/or cytostatic activity against cells expressing PSMA. The antibody-drug conjugate compounds comprise an Antibody unit covalently linked to at least one Drug unit. The Drug units can be covalently linked directly or via a Linker unit (-LU-).

In some embodiments, the antibody-drug conjugate compound has the following formula:

Ab-(LU-D)p
or a pharmaceutically acceptable salt or solvate thereof; wherein:
Ab is the Antibody unit, e.g., Mut-gy1 or its mutated variants derived full antibody or antigen-binding fragment thereof of the present invention, such as PSMAb, and
(LU-D) is a Linker unit-Drug unit moiety, wherein:
LU- is a Linker unit, and
-D is a drug unit having cytostatic or cytotoxic activity against a target cell; and
p is an integer from 1 to 20.

In some embodiments, p ranges from 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is 1, 2, 3, 4, 5 or 6. In some embodiments, p is 2 or 4.

In some embodiments, the antibody-drug conjugate compound has the following formula:

Ab-(Aₐ-W_{w}Y_{y}-D)ₚ
or a pharmaceutically acceptable salt or solvate thereof, wherein:
Ab is the Antibody unit, e.g., Mut-gy1 or its mutated variants derived full antibody or antigen-binding fragment thereof of the present invention, such as PSMAb; and
-Aₐ-W_{w}-Y_{y} is a Linker unit (LU), wherein:
-A- is a Stretcher unit,
a is 0 or 1,
each -W- is independently an Amino Acid unit,
w is an integer ranging from 0 to 12,
-Y- is a self-immolative spacer unit,
y is 0, 1 or 2;
-D is a drug units having cytostatic or cytotoxic activity against the target cell; and
p is an integer from 1 to 20.

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 10, 1 to 9, 1 to 8, 1 to 7, 1 to 6, 1 to 5, 1 to 4, 1 to 3, or 1 to 2. In some embodiments, p ranges from 2 to 8, 2 to 7, 2 to 6, 2 to 5, 2 to 4 or 2 to 3. In other embodiments, p is 1, 2, 3, 4, 5 or 6. In some embodiments, p is 2 or 4. In some embodiments, when w is not zero, y is 1 or 2. In some embodiments, when w is 1 to 12, y is 1 or 2. In some embodiments, w is 2 to 12, and y is 1 or 2. In some embodiments, a is 1 and w and y are 0.

For compositions comprising a plurality antibodies, the drug loading is represented by p, the average number of drug molecules per Antibody. Drug loading may range from 1 to 20 drugs (D) per Antibody. The average number of drugs per antibody in preparation of conjugation reactions may be characterized by conventional means such as mass spectroscopy, ELISA assay, and HPLC. The quantitative distribution of Antibody-Drug-Conjugates in terms of p may also be determined. In some instances, separation, purification, and characterization of homogeneous Antibody-Drug-conjugates where p is a certain value from Antibody-Drug-Conjugates with other drug loadings may be achieved by means such as reverse phase HPLC or electrophoresis. In exemplary embodiments, p is from 2 to 8.

The generation of Antibody-drug conjugate compounds can be accomplished by any technique known to the skilled artisan. Briefly, the Antibody-drug conjugate compounds comprise Mut-gy1 or its mutated variants derived full antibody or antigen-binding fragment thereof of the present invention as the Antibody unit, a drug, and optionally a linker that joins the drug and the binding agent. In a preferred embodiment, the Antibody is antibody or antigen-binding fragment thereof derived from Mut-gy1 or its variants with point mutations, as described elsewhere herein. A number of different reactions are available for covalent attachment of drugs and/or linkers to binding agents. This is often accomplished by reaction of the amino acid residues of the binding agent, e.g., antibody molecule, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of an antibody molecule. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates can also be used as coupling agents for covalently attaching drugs to binding agents. Other techniques are known to the skilled artisan and within the scope of the present invention.

In some embodiments, an intermediate, which is the precursor of the linker, is reacted with the drug under appropriate conditions. In some embodiments, reactive groups are used on the drug and/or the intermediate. The product of the reaction between the drug and the intermediate, or the derivatized drug, is subsequently reacted with the Mut-gy1 or its variants derived full antibody or antigen-binding fragment thereof under appropriate conditions.

### PSMA-Targeted CAR-T or CAR-NK

Recent developments using chimeric antigen receptor (CAR) modified autologous T cell (CART) therapy, which relies on redirecting T cells to a suitable cell-surface molecule on cancer cells such as B cell malignancies, show promising results in harnessing the power of the immune system to treat B cell malignancies and other cancers (see, e.g., Sadelain et al., Cancer Discovery 3:388-398 (2013)). The clinical results of the murine derived CART19 (i.e., “CTL019”) have shown promise in establishing complete remissions in patients suffering with CLL as well as in childhood ALL (see, e.g., Kalos et al., Sci Transl Med 3:95ra73 (2011), Porter et al., NEJM 365:725-733 (2011), Grupp et al., NEJM 368:1509-1518 (2013)). Besides the ability of the chimeric antigen receptor on the genetically modified T cells to recognize and destroy the targeted cells, a successful therapeutic T cell therapy needs to have the ability to proliferate and persist over time, and to further monitor for leukemic cell escapees. The variable quality of T cells, whether it's a result of energy, suppression or exhaustion, will have effects on CAR-transformed T cells' performance but for which skilled practitioners have limited control over at this time. To be effective, CAR-transformed patient T cells need to persist and maintain the ability to proliferate in response to the CAR's antigen. It has been shown that ALL patient T cells can do this with CART19 comprising a murine scFv (see, e.g., Grupp et al., NEJM 368:1509-1518 (2013)).

The invention addresses controlling an immune response in patients by providing fully human antibody fragments (e.g., scFv) that bind PSMA integrated into a Chimeric Antigen Receptor (CAR) construct that will redirect the engineered T cell to recognize and kill PSMA positive tumor cells.

Accordingly, in one aspect, the invention pertains to an isolated nucleic acid molecule encoding a chimeric antigen receptor (CAR), wherein the CAR comprises an antibody or antigen-binding fragment thereof, which includes a PSMA binding domain, a transmembrane domain, and an intracellular signaling domain (e.g., an intracellular signaling domain comprising a costimulatory domain and/or a primary signaling domain). In one embodiment, the CAR comprises an antibody or antigen-binding fragment thereof, which includes a fully human anti-PSMA binding domain described herein, a transmembrane domain described herein, and an intracellular signaling domain described herein (e.g., an intracellular signaling domain comprising a costimulatory domain and/or a primary signaling domain).

In one embodiment, the encoded human anti-PSMA binding domain comprises one or more (e.g., all three) light chain complementary determining region 1 (LC CDR1), light chain complementary determining region 2 (LC CDR2), and light chain complementary determining region 3 (LC CDR3) of a fully human anti-PSMA binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a fully human anti-PSMA binding domain described herein, e.g., a fully human anti-PSMA binding domain comprising one or more, e.g., all three, LC CDRs and/or one or more, e.g., all three, HC CDRs. In one embodiment, the encoded light chain variable region comprises one, two, three or all four framework regions described herein. In one embodiment, the encoded heavy chain variable region comprises one, two, three or all four framework regions described below. In one embodiment, the encoded fully human anti-PSMA binding domain comprises a human light chain variable region described below and/or a human heavy chain variable region described below. In one embodiment, the encoded anti-PSMA binding domain is a scFv comprising a light chain and a heavy chain of an amino acid sequence described below. In an embodiment, the anti-PSMA binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided below, or a sequence with 95-99% identity with an amino acid sequence described below; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided below, or a sequence with 95-99% identity to an amino acid sequence described below. In one embodiment, the encoded human anti-PSMA binding domain comprises a sequence described below, or a sequence with 95-99% identity thereof. In one embodiment, the nucleic acid sequence encoding the human anti-PSMA binding domain comprises a sequence described below, or a sequence with 95-99% identity thereof. In one embodiment, the encoded human anti-PSMA binding domain is a scFv, and a light chain variable region is attached to a heavy chain variable region via a linker, e.g., a linker described herein. In one embodiment, the encoded human anti-PSMA binding domain includes a (Gly4-Ser)n linker, wherein n is 1, 2, 3, 4, 5, or 6, preferably 3 or 4. In another embodiment, the encoded human anti-PSMA binding domain contains a linker sequence as described in SEQ ID NO: 37. The light chain variable region and heavy chain variable region of a scFv can be, e.g., in any of the following orientations: light chain variable region-linker-heavy chain variable region or heavy chain variable region-linker-light chain variable region.

In some embodiments, the anti-PSMA binding domain comprises an antibody or antigen-binding fragment thereof described elsewhere herein. For example, in some embodiments, the anti-PSMA binding domain comprises Mut-gy1, Mut-gy1-st, Mut-gy1-2, Mut-gy1-3 or PSMAbLm, as described elsewhere herein.

### PSMA Targeted Bispecific Antibodies

Different from natural antibodies, bispecific antibodies (BsAbs) are artificial antibodies that bear double specificities, targeting two different tumor antigens, or one on tumor cells and the other on effector cells that can recruit the immunological effector cells to tumor sites efficiently and activate them to kill tumor cells specifically. Examples for the former, i.e., BsAbs targeting two different tumor associated antigens are bH1, which binds Her2 and VEGF simultaneously (Bostrom J1 et al. 2009, Science 323: 1610-4), or ErBb2/ErBb3 dual targeting bispecific scFv (Robinson M K et al. 2008, Br J Cancer 99:1415-25). The two tumor-associated antigens could both be expressed on tumor cells, or one expressed on tumor cells and the other on tumor cell-associated cells, such as tumor microenvironment cells, e.g., fibroblast, vascular cells, endothelium, pericytes or immune cells in tumor microenvironment (macrophage, B cells, T cells etc.). Examples for the latter, i.e., BsAbs targeting one tumor-associated antigen and an immunoactiavable antigen are BsAbs with one arm targeting a tumor antigen, such as Her2, CD19, or CD123 and the other arm targeting an immunoactivatable antigen, such as CD3 or CD16 (Kontermann R E, et al. 2015, Drug Discovery Today 20: 838-847) that could engage tumor cells and immune cells such as T cells, NK cells or macrophage etc.

A BsAb containing an anti-CD3 antibody will engage T cells and tumor cells together, leading to killing of the tumor cells (Muller and Kontermann, BioDrugs 2010; 24: 89-98, Baeuerle and Reinhardt 2009, Cancer Research 96: 4941) activated T cells. Blinatumomab (Bargou et al., Science 2008, 321: 974-976) is a single chain antibody construct named BiTE, which induces cytotoxicity by targeting CD19 and CD3. Other antibody fragment-based T-cell engaging bispecifics have been described (Moore et al. 2011, Blood 117:4542-4551, Baeuerle et al. Current Opinion in Molecular Therapeutics 2009, 11: 22-30). The BiTE™ format is a bi-specific single chain antibody construct that links variable domains derived from two different antibodies. Blinatumomab, however, possesses a poor half-life in vivo, and is difficult to manufacture in terms of production and stability. Thus, there is a need for improved bi-specific antibodies capable of targeting T-cells to tumor cells and having improved manufacturability.

BsAbs are hybrid proteins that can be generated by chemical cross-link, hybridoma technology or genetic methods. In the chemical cross-link method, two kinds of monoclonal antibodies and fragments thereof were dissociated by reductants to generate monovalent antibodies and fragments thereof. The resulting BsAb is constructed via chemical cross-linking of two monovalent antibodies and fragments thereof from different parental antibodies. This strategy can be used for rapid production of BsAb in a large scale but BsAb can be inactivated sometimes during cross-link, and it is difficult to guarantee the homogeneity of products. Another strategy for production of BsAb is hybridoma technology by which an established hybridoma cell line secreting one monoclonal antibody was fused to spleen cells immunized with the other antigen or two established hybridoma cell lines secreting two different monoclonal antibodies were fused each other to create hybrid hybridomas. The former resulting hybridoma is called dimeric hybridoma and tetrameric hybridoma. Generally, BsAb produced by hybridoma technology keeps high bioactivities. However, the procedures are tedious and time-consuming, and it is not easy to isolate BsAb from other non-active and unwanted antibodies generated simultaneously. These BsAb formats encountered another predictable problems: too large size and murine components contained in BsAb are immunogenic in patients and will induce the production of human anti-mouse antibodies (HAMA), which may prevent reuse of these BsAbs in the clinic. Furthermore, production and purification of these formats of BsAb are expensive, which limits the application of BsAbs in clinic. Replacement of these traditional methods with gene recombination approaches has accelerated progress in this area. Based on the technology of small molecular antibodies, production of BsAb by gene engineering has advantages over those described above, such as the stability of process, large scale production, low cost, and easy-to-use. Gene engineering has led to the development of various small molecular BsAb formats by connecting two different kinds of scFvs. There are three kinds of BsAb formats classified by different links. (1) mini-antibodies are heterodimers assembled by connecting two scFv fragments together with an oligomerized domain (e.g., leucine zipper motifs derived from Fos or Jun transcription factors). (2) Diabodies are non-covalently associated dimmers which are assembled by two single chains VH1-VL2 and VH2-VL1, both connected by a short linker that is too short to allow pairing between V-domains from the same chain. Thus, each chain alone is not capable of binding antigen, but co-expression of two chains (VH1-VL2 and VH2-VL1) leads to assembly of heterodimeric diabodies which can bind to two kinds of antigens. (3) ScBsAb: an interlinker was used for connecting two different scFvs with different specificities and ScBsAb was expressed in the host cells as a single polypeptide. The intralinker between two domains within scFv is often (Gly4Ser)3. As for the interlinker between two scFvs, there are two strategies for designing it. For the purpose of avoiding false paring between heterogenous variable regions, the interlinker is often a short peptide linker with less than ten amino acid residues, such as Gly4Ser. Another strategy is to select a longer linker for the interlinker. In a word, the most important for designing interlinkers is to ensure the proper pairing between variable domains and folding of proteins, resulting in the formation of BsAb which maintains biological activities and stability. Some novel properties for facilitating purification and extending the plasma half-life time should be introduced.

BsAb-mediated immunotherapy plays a promising role in the clinical biotherapy for tumors. Tumor-killing effects mediated by BsAb is based on stimulating the immune system, highly specific with tumors and free of MHC restriction. Therefore, BsAb-mediated therapy is the complementarity of traditional methods such as surgery, radiotherapy and chemotherapy. BsAb can not only cure tumors but also stimulate the immune system to provide and keep the immune protection for a long time. Based on results of experiments in mouse and clinic, optimal BsAb prepared for trial use should have at least five characteristics as follows: 1): It targets to the relevant tumor antigens with high specificity and affinity; 2): It can bind to trigger factors on effector cells-cytotoxic cells and result in cross link only when BsAb binds to tumor antigens; 3): BsAb is able to promote the effective cytotoxicity and inflammation selectively produced by the corresponding group of leukocytes at tumor sites; 4): BsAb must be humanized to minimize induction of human anti-mouse response following repeated uses; Finally, 5): BsAb should be not only small enough to penetrate into tumors but also large enough to keep in the circulation for a sufficient time.

Based on these points described above, numerous BsAbs triggering many kinds of immune effector cells and targeting different tumor cells have been developed in the past few years, wherein the effector cells include T lymphocytes, NK cells, monocytes, macrophages, neutrophils, LAK cells (lymphokine-activated cytotoxic cells) and TIL cells (tumor infiltrating lymphocytes) etc. T cells are commonly recognized as the major specific cells for immune responses. CD3 expressed on the surface of all mature T cells is the common surface marker for T cells. CD3 binds to TCR non-covalently, forming the whole TCR-CD3 complex, and involves in immune responses against antigen stimulus. Now CD3 is surface trigger molecule on immune effector cells used most widely and successfully. Following anti-CD3 antibody within BsAb binds to CD3 molecule on the surface of T cells, numerous effects as follows will be produced to kill tumor cells. These effects include: (1) proliferation and differentiation of T cells. Firstly, BsAb can activate the rest T cells, resulting in Th cell and Tc cell derived from the premature effector T cells with CD4+ or CD8+. Secondly, BsAb can activate numerous memory cells to proliferate and differentiate into effector T cells which will attack and kill tumor cells. The number of effector cells is directly related to the rate of tumor elimination. (2) release of cytokines: CD4+Th cells activated by BsAb can secrete a great deal of IL-2. IL-2 not only stimulates the proliferation of Th cells in autocrine, but also activates naive CD8+ T cells in paracrine to become Tc cells, resulting in enlargement of cytotoxicity of Tc cells. In addition, IL-2 is a costimulating signal for activating T cells. Therefore, IL-2 plays a vital role in BsAb-mediated immune effects. Some other cytokines, such as TNF-α and IFN-γ are produced in the process of T-cell activation and can produce ‘stander-by’ effect by inhibiting the growth of ‘stander-by’ tumor cells through the medium among cells. (3) cytotoxicity: In vitro experiments indicate that mediated by BsAb, CD8+Tc interacts with tumor cells directly, releases cytotoxic materials through granule exocytosis and lyses target cells, which takes place rapidly usually within 4-6 hours following targeting tumor cells. The major components in the cytotoxic materials are perforin and serine easterases or granzymes. Perforins can attack the plasma membrane and form ion channels, thus causing entry of plenty of ions and water, resulting in the lysis and necrosis of cells while granzymes are similar to lymphotoxin, capable of activating DNases in the cell, thus causing lysis of nucleic DNA, resulting in the apoptosis of target cells.

Currently, Fv fragments are widely used for construction of BsAb, since they are the minimal unit with the complete antigen-binding site, small (about ⅙ of the whole antibody), absence of Fc domain, lower immunogenicity, easily penetration into the wall of blood vessels and solid tumors. However, Fv is unstable and easy to dissociate in vivo because the covalent bond between VH and VL domains is unable to generate. In order to improve the stability of Fv fragment, a polypeptide intralinker between VH and VL domains is used to form so-called ScFv. The intralinker is commonly a short flexible peptide with 15 amino acid residues in length such as (Gly₄Ser)₃. In one embodiment of the present invention, the intralinker was used in anti-CD3 ScFv, and a different intralinker is used for anti-PSMA scFv.

Bispecific antibodies having a standard IgG format can be challenging to produce because they include four different polypeptide chains. The efficacy of a smaller, more easily-produced bispecific molecule has been clinically demonstrated in non-Hodgkin's lymphoma. See, e.g., Bargou et al. (2008), Science 321(5891): 974-977.

Prolonged administration by continuous intravenous infusion was used to achieve these results because of short in vivo half-life of this small, single chain molecule. Hence, there is a need in the art for bispecific therapeutics that retain similar therapeutic efficacy, that have a format that is straightforward to produce, and that have favorable pharmacokinetic properties, including a longer half-life.

A Bispecific-Fc (Bs-Fc), as described herein, can bind to two different proteins and contains an Fc region of an antibody or a portion thereof. A Bs-Fc can have favorable pharmacokinetic properties relative to a bispecific single-chain molecule lacking an Fc region. One protein bound by a Bs-Fc can be expressed on an immune effector cell such as a T cell, an NK cell, a neutrophil, or a macrophage, and the other protein can be expressed on a target cell, for example, a cancer cell, a cell infected by a pathogen, or a cell mediating a disease, such as a fibroblast causing fibrosis. The Bs-Fc molecules described herein can elicit activation of an immune effector cell in the presence of a target cell and/or killing of a target cell in the presence of an immune effector cell.

In some embodiments, the anti-PSMA binding domain comprises an antibody or antigen-binding fragment thereof described elsewhere herein. For example, in some embodiments, the anti-PSMA binding domain comprises Mut-gy1, Mut-gy1-st, Mut-gy1-2, Mut-gy1-3 or PSMAbLm, as described elsewhere herein.

### Methods of Treatment

In another aspect, this disclosure further provides a method of preventing or treating a proliferative disease associated with expression of PSMA in a subject. In some embodiments, the method comprises administering to the subject an effective amount of the antibody or antigen-binding fragment thereof or variant thereof, the bispecific or multiple-specific antibody or antigen-binding fragment thereof, the CAR, the nucleic acid molecule, the vector, the cell, or the composition, as described herein. In some embodiments, the subject is a mammal, e.g., a human.

In some embodiments, the method further comprises administering to the subject a second agent or therapy. In some embodiments, the second agent or therapy comprises an anti-cancer agent. In some embodiments, the second agent or therapy is administered to the subject before, after, or concurrently with the antibody or antigen-binding fragment thereof or variant thereof, the bispecific or multiple-specific antibody or antigen-binding fragment thereof, the CAR, the nucleic acid molecule, the vector, the cell, or the composition, as described herein.

In one embodiment, the invention provides a method of treating a solid tumor with abnormal PSMA expression, such as prostate cancer or solid tumors with high PSMA expression in neovasculature, in a subject comprising the step of targeting PSMA high expression cells with the antibody or antigen-binding fragment. In some embodiments, the method comprises administering to a subject having a tumor associated with high PSMA expression, a composition comprising an antibody or antigen-binding fragment thereof.

In one embodiment, the antibody or antigen-binding fragment thereof is operably linked to a biologically active agent or a combination of such agents, wherein the agent is a toxin, a radioisotope, a nanoparticle or a bioactive peptide. These tumors include but not limited to prostate cancer, lung cancer, liver cancer, pancreas cancer, colon cancer, gastric cancer, breast cancer, ovarian cancer, kidney cancer, prostate cancer, bladder cancer, melanoma, glioma etc.

In one embodiment, provided herein is a method of inhibiting or suppressing a tumor in a subject. In another embodiment, the method comprises the step of administering an effective amount of the antibody or antigen-binding fragment described herein.

In another embodiment, provided herein is a method of delaying progression of a solid tumor in a subject. In yet another embodiment, the method comprises administering to the subject an effective amount of the antibody or antigen-binding fragment thereof provided herein. In another embodiment, the subject mounts an immune response against a vasculature of the solid tumor, thereby delaying progression of the solid tumor in the subject.

In one embodiment, the term “operably linked” refers to the positioning/linking of the two or more molecules or sequences in such a manner as to ensure the proper function or expression of the molecule and sequence.

In one embodiment, the term “therapeutically effective amount” refers to an amount that provides a therapeutic effect for a given condition and administration regimen. In the present invention, the therapeutic effect is the prevention or inhibition of tumor growth, infiltration, spread, metastasis or relapse, or preferably reduction of tumor burden, or the improvement of patient outcome.

In one embodiment, the term “preventing or treating” refers to any one or more of the following: delaying the onset of symptoms, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, preventing relapse to a disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics. In one embodiment, “treating” refers to both therapeutic treatment and prophylactic or preventive measures, wherein the object is to prevent or lessen the targeted pathologic condition or disorder as described hereinabove.

In another embodiment, “symptoms” are manifestation of a disease or pathological condition as described hereinabove.

In one embodiment, the compositions of this invention comprise a polypeptide, antibody, or antigen-binding fragment of this invention, alone or in some embodiments, in combination with a second pharmaceutically active agent. In another embodiment, the composition further comprises a proteolytic inhibitor, pharmaceutical carrier, diluent, and adjuvant.

In one embodiment, the term “pharmaceutically active agent” refers to any medicament which satisfies the indicated purpose. In some embodiments, the pharmaceutically active agent includes, but is not limited to a chemotherapeutic drug, radiotherapy drug, angiogenesis inhibitor, tumor imaging probe, immune modulator, or any other tumor therapy and/or imaging drug/agent, and the like.

In another embodiment, provided herein is a method of delivering a biologically active agent and the antibody or antigen-binding fragment of the present invention for the treatment of a tumor in a subject. In another embodiment, the method comprises the step of concomitantly but individually administering the biologically active agent and the antibody or antigen-binding fragment. In another embodiment, the method comprises the step of separately administering the biologically active agent and the antibody or antigen-binding fragment.

In one embodiment, the antibody or antigen-binding fragment provided herein are themselves “biologically active,” meaning they are able to exert the biological action or an enhanced action of their corresponding parental antibodies even after modification, in particular in binding to the target antigen, inhibiting binding of ligands to receptors, further in terms of modulation, in particular inhibition of antigen-mediated signal transduction and prophylaxis or therapy of antigen-mediated diseases. The term “biologically active,” when used in reference to any of the biologically active agents described herein, also refers to the agent's ability to modulate the immune response in a manner that can lead to a preventive, diagnostic, or therapeutic effect as will be understood by a skilled artisan. In some embodiments, agents that are used to achieve this biological activity include but are not limited to a cytokine, an enzyme, a chemokine, a radioisotope, an enzymatically active toxin, a therapeutic nanoparticle, or a chemotherapeutic agent, as will be understood by a skilled artisan.

In an alternate embodiment, the polypeptides of antibodies are conjugated or operably linked so as to function in their intended purpose to an enzyme in order to employ Antibody Dependent Enzyme Mediated Prodrug Therapy (ADEPT). ADEPT may be used by conjugating or operably linking the antibody or Fc fusion to a prodrug-activating enzyme that converts a prodrug (e.g., a peptidyl chemotherapeutic agent) to an active anti-cancer drug. The enzyme component of the immunoconjugate useful for ADEPT includes any enzyme capable of acting on a prodrug in such a way so as to convert it into its more active, cytotoxic form. Other additional modifications of the modified molecules provided herein are contemplated herein. For example, the polypeptide/antibody may be linked to one of a variety of nonproteinaceous polymers, e.g., polyethylene glycol (PEG), polypropylene glycol, polyoxyalkylenes, or copolymers of polyethylene glycol and polypropylene glycol.

In another embodiment, the antibody/polypeptide provided herein is administered with one or more immunomodulatory agents. Such agents may increase or decrease production of one or more cytokines, up- or down-regulate self-antigen presentation, mask MHC antigens, or promote the proliferation, differentiation, migration, or activation state of one or more types of immune cells. Immunomodulatory agents include but are not limited to: non-steroidal anti-inflammatory drugs (NSAIDs) such as aspirin, ibuprofen, celecoxib, diclofenac, etodolac, fenoprofen, indomethacin, ketoralac, oxaprozin, nabumentone, sulindac, tolmentin, rofecoxib, naproxen, ketoprofen, and nabumetone; steroids (e.g., glucocorticoids, dexamethasone, cortisone, hydroxycortisone, methylprednisolone, prednisone, prednisolone, trimcinolone, azulfidineicosanoids such as prostaglandins, thromboxanes, and leukotrienes; as well as topical steroids such as anthralin, calcipotriene, clobetasol, and tazarotene); cytokines such as TGFβ, IFNα, IFNβ, IFNγ, IL-2, IL-4, IL-10; cytokine, chemokine, or receptor antagonists including antibodies, soluble receptors, and receptor-Fc fusions against BATF, B7, CCR2, CCR5, CD2, CD3, CD4, CD6, CD7, CD8, CD11, CD14, CD15, CD17, CD18, CD20, CD23, CD28, CD40, CD40L, CD44, CD45, CD52, CD64, CD80, CD86, CD147, CD152, complement factors (C5, D) CTLA4, eotaxin, Fas, ICAM, ICOS, IFN-α IFN-β, IFN-γ, IFNAR, IgE, IL-1, IL-2, IL-2R, IL-4, IL-5R, IL-6, IL-8, IL-9 IL-12, IL-13, IL-13R1, IL-15, IL-18R, IL-23, integrins, LFA-1, LFA-3, MHC, selectins, TGF-β, TNF-α, TNF-β, TNF-R1, T-cell receptor, including Enbrel®. (etanercept), Humira®. (adalimumab), Remicade® (infliximab), PD1 antibodies (OPDIVO® (nivolumab), KEYTRUDA® (pembrolizumab)) or PD-L1 antibodies (durvalumab, MPDL3280A); heterologous anti-lymphocyte globulin; other immunomodulatory molecules such as 2-amino-6-aryl-5 substituted pyrimidines, anti-idiotypic antibodies for MHC binding peptides and MHC fragments, azathioprine, brequinar, bromocryptine, cyclophosphamide, cyclosporine A, D-penicillamine, deoxyspergualin, FK506, glutaraldehyde, gold, hydroxychloroquine, leflunomide, malononitriloamides (e.g., leflunomide), methotrexate, minocycline, mizoribine, mycophenolate mofetil, rapamycin, and sulfasasazine.

In an alternate embodiment, antibodies of the present invention are administered with a cytokine. By “cytokine” as used herein is meant as a generic term for proteins released by one cell population that act on another cell as intercellular mediators. Examples of such cytokines are lymphokines, monokines, and traditional polypeptide hormones. Included among the cytokines are fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and —II; erythropoietin (EPO); osteoinductive factors; interferons such as interferon-alpha, beta, and -gamma; colony-stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

A chemotherapeutic or other cytotoxic agent may be administered as a prodrug. The term “prodrug” refers to a precursor or derivative form of a pharmaceutically active substance that is less cytotoxic to tumor cells compared to the parent drug and is capable of being enzymatically activated or converted into the more active parent form. See, for example, Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985. The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies/polypeptides of the compositions and methods provided herein include but are not limited to any of the aforementioned chemotherapeutic agents.

In some embodiments, any combination of the antibody/polypeptide with the biological active agents specified above, i.e., a cytokine, an enzyme, a chemokine, a radioisotope, an enzymatically active toxin, or a chemotherapeutic agent can be applied. In another embodiment, the antibody/polypeptide can be operably linked with the biologically active agent and used in the methods described herein or antibody/polypeptide provided herein can merely be used in combination with the biologically active agents, in a manner in which both are administered separately (i.e., not conjugated) to achieve the desired preventive, diagnostic, or therapeutic effect.

### Combination Therapies

A composition comprising an antibody, antibody fragment, ADC, CAR-expressing cell, or bispecific antibody described herein may be used in combination with other known agents and therapies. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A composition comprising an antibody, antibody fragment, ADC, CAR-expressing cell, or bispecific antibody described herein, and at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

In further aspects, a composition comprising an antibody, antibody fragment, ADC, CAR-expressing cell, or bispecific antibody described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation. Peptide vaccine, such as that described in Izumoto et al. 2008 J Neurosurg 108:963-971.

In one embodiment, a composition comprising an antibody, antibody fragment, ADC, CAR-expressing cell, or bispecific antibody described herein can be used in combination with a chemotherapeutic agent. Exemplary chemotherapeutic agents include an antiandrogen (androgen antagonists), anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, dacarbazine, melphalan, ifosfamide, temozolomide), an immune cell antibody (e.g., alemtuzumab, gemtuzumab, rituximab, tositumomab), an antimetabolite (including, e.g., folic acid antagonists, pyrimidine analogs, purine analogs, and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Exemplary Antiandrogen (androgen antagonists) agents include Bicalutamide, Goserelin Acetate SR Depo.

Exemplary alkylating agents include, without limitation, nitrogen mustards, ethylenimine derivatives, alkyl sulfonates, nitrosoureas, and triazenes): uracil mustard (Aminouracil Mustard®, Chlorethaminacil®, Demethyldopan®, Desmethyldopan®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexylen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexylen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary mTOR inhibitors include, e.g., temsirolimus; ridaforolimus (formally known as deferolimus, (1R,2R,4S)-4-[(2R)-2[(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon 7, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine, and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.:WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, the subject can be administered an agent which reduces or ameliorates a side effect associated with the administration of a naked antibody or antigen-binding fragment thereof, ADC, CAR-expressing cell, or Bispecific antibody.

In one embodiment, the subject can be administered an agent which enhances the activity of a naked antibody or antigen-binding fragment thereof, ADC, CAR-expressing cell, or Bispecific antibody. For example, in one embodiment, the agent can be an agent which inhibits an inhibitory molecule. Inhibitory molecules, e.g., Programmed Death 1 (PD1), can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, and TGFR beta. Inhibition of an inhibitory molecule, e.g., by inhibition at the DNA, RNA or protein level, can optimize a naked antibody or antigen-binding fragment thereof, ADC, CAR-expressing cell, or Bispecific antibody performance. In embodiments, an inhibitory nucleic acid, e.g., an inhibitory nucleic acid, e.g., a dsRNA, e.g., a siRNA or shRNA, can be used to inhibit expression of an inhibitory molecule in the CAR-expressing cell or bispecific Ab reacting cells. In an embodiment, the inhibitor is an shRNA. In one embodiment, the inhibitor of an inhibitory signal can be, e.g., an antibody or antigen-binding fragment thereof that binds to an inhibitory molecule. For example, the agent can be an antibody or antigen-binding fragment thereof that binds to PD1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206).). In an embodiment, the agent is an antibody or antigen-binding fragment thereof that binds to TIM3. In an embodiment, the agent is an antibody or antigen-binding fragment thereof that binds to LAG3.

PD1 is an inhibitory member of the CD28 family of receptors that also includes CD28, CTLA-4, ICOS, and BTLA. PD1 is expressed on activated B cells, T cells, and myeloid cells (Agata et al. 1996 Int. Immunol 8:765-75). Two ligands for PD1, PD-L1, and PD-L2 have been shown to downregulate T cell activation upon binding to PD 1 (Freeman et al. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1. Antibodies, antibody fragments, and other inhibitors of PD1, PD-L1, and PD-L2 are available in the art and may be used in combination with a PSMA targeted naked antibody or antigen-binding fragment thereof, ADC, CAR-expressing cell, or Bispecific antibody described herein. For example, nivolumab (also referred to as BMS-936558 or MDX1106; Bristol-Myers Squibb) is a fully human IgG4 monoclonal antibody that specifically blocks PD 1. Nivolumab (clone 5C4) and other human monoclonal antibodies that specifically bind to PD1 are disclosed in U.S. Pat. No. 8,008,449 and WO2006/121168. Pidilizumab (CT-011; Cure Tech) is a humanized IgG1k monoclonal antibody that binds to PD1Pidilizumab and other humanized anti-PD1 monoclonal antibodies are disclosed in WO2009/101611. Lambrolizumab (also referred to as MK03475; Merck) is a humanized IgG4 monoclonal antibody that binds to PD1. Lambrolizumab and other humanized anti-PD1 antibodies are disclosed in U.S. Pat. No. 8,354,509 and WO2009/114335. MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. Other anti-PD-L1 binding agents include YW243.55.570 (heavy and light chain variable regions are shown in SEQ ID NOs 20 and 21 in WO2010/077634) and MDX-1 105 (also referred to as BMS-936559, and, e.g., anti-PD-L1 binding agents disclosed in WO2007/005874). AMP-224 (B7-DCIg; Amplimmune; e.g., disclosed in WO2010/027827 and WO2011/066342) is a PD-L2 Fc fusion soluble receptor that blocks the interaction between PD1 and B7-H1. Other anti-PD1 antibodies include AMP 514 (Amplimmune), among others, e.g., anti-PD1 antibodies disclosed in U.S. Pat. No. 8,609,089, US 2010028330, and/or US 20120114649.

In some embodiments, the agent which enhances the activity of a CAR-expressing cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the CAR. In another embodiment, the fusion protein is expressed by a cell, e.g., a T cell that does not express an anti-PSMA CAR.

### Formulations and Route of Administration

The antibodies, ADC, CAR-expressing cell, or bispecific antibody of the present invention may find use in a wide range of products. In one embodiment, the antibody, ADC, CAR-expressing cell, or bispecific antibody of the invention is a therapeutic, a diagnostic, or a research reagent. In one embodiment, an antibody ADC, CAR-expressing cell, or bispecific antibody of the invention is a therapeutic. In some embodiments, antibody or antigen-binding fragment thereof, ADC, CAR-expressing cell, or bispecific antibody of the present invention is used for industrial uses. An antibody of the present invention may find use in an antibody composition that is monoclonal or polyclonal. The antibodies of the present invention may be agonists, antagonists, neutralizing, inhibitory, or stimulatory. In one embodiment, the antibodies or antibody fragments, ADC, CAR-expressing cell, or bispecific antibody of the present invention are used to kill target cells that bear the target antigen, for example, cancer cells. In an alternate embodiment, the antibodies of the present invention are used to block, antagonize, or agonize the target antigen. In an alternate embodiment, the antibodies of the present invention are used to block, antagonize, or agonize the target antigen and kill the target cells that bear the target antigen. In another embodiment, the target cell is a tumor cell or its neovasculature. In one embodiment, neovasculature plays an important role in angiogenesis and is also considered to be a target of the antibodies or antibody fragments, ADC, CAR-expressing cell, or Bispecific antibody provided herein.

This disclosure further provides kits comprising one or more compositions of the invention, including pharmaceutical formulations, packaged into suitable packaging material. In another embodiment, a kit includes a nucleic acid encoding the antibody or antigen-binding fragments, Car T or Car NK cells, or bispecific antibody, thereof of the invention. In additional embodiments, a kit includes nucleic acids that further include an expression control element; an expression vector; a viral expression vector; an adeno-associated virus expression vector; an adenoviral expression vector; and a retroviral expression vector. In yet an additional embodiment, a kit includes a cell that expresses the antibody or antigen-binding fragments thereof of the invention, such as the Car T or Car NK cells.

In additional embodiments, a kit includes a label or packaging insert including instructions for expressing an antibody or bispecific antibody or a nucleic acid encoding the antibody, antigen-binding fragments or bispecific antibody thereof in cells in vitro, in vivo, or ex vivo. In yet additional embodiments, a kit includes a label or packaging insert including instructions for treating a subject (e.g., a subject having or at risk of having asthma) with the antibody or antigen-binding fragment thereof, ADC, CAR-expressing cell, or Bispecific antibody thereof of the invention in vivo, or ex vivo.

As used herein, the term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention, e.g., treating the common cold. The kits can additionally include instructions for using the kit components in a method of the invention.

Instructions can include instructions for practicing any of the methods of the invention described herein. Thus, invention pharmaceutical compositions can be included in a container, pack, or dispenser together with instructions for administration to a subject. Instructions may additionally include indications of a satisfactory clinical endpoint or any adverse symptoms that may occur, or additional information required by the Food and Drug Administration for use on a human subject.

In one embodiment, polypeptides of the present invention are administered as part of a vaccine. In some embodiments, the term vaccine is to be understood to encompass any immunomodulating composition, and such vaccines may comprise an adjuvant, an antigen, an immunomodulatory compound, or a combination thereof, in addition to the polypeptides of this invention.

In some embodiments, an adjuvant may include, but is not limited to: (A) aluminium compounds (e.g. aluminium hydroxide, aluminium phosphate, aluminium hydroxyphosphate, oxyhydroxide, orthophosphate, sulphate, etc. [e.g. see chapters 8 & 9 of ref. 96]), or mixtures of different aluminium compounds, with the compounds taking any suitable form (e.g. gel, crystalline, amorphous, etc.), and with adsorption being preferred; (B) MF59 (5% Squalene, 0.5% Tween 80, and 0.5% Span 85, formulated into submicron particles using a microfluidizer); (C) liposomes; (D) ISCOMs, which may be devoid of additional detergent; (E) SAF, containing 10% Squalane, 0.4% Tween 80, 5% pluronic-block polymer L121, and thr-MDP, either micro fluidized into a submicron emulsion or vortexed to generate a larger particle size emulsion; (F) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (G) saponin adjuvants, such as QuilA or QS21, also known as Stimulon™; (H) chitosan; (I) complete Freund's adjuvant (CFA) and incomplete Freund's adjuvant (IFA); (J) cytokines, such as interleukins (e.g. IL-1, IL-2, IL-4, IL-5, IL-6, IL-7, IL-12, etc.), interferons (e.g. interferon-γ), macrophage colony stimulating factor, tumor necrosis factor, etc.; (K) monophosphoryl lipid A (MPL) or 3-O-deacylated MPL (3dMPL)]; (L) combinations of 3dMPL with, for example, QS21 and/or oil-in-water emulsions; (M) oligonucleic acids comprising CpG motifs]i.e. containing at least one CG dinucleic acid, with 5-methylcytosine optionally being used in place of cytosine; (N) a polyoxyethylene ether or a polyoxyethylene ester; (O) a polyoxyethylene sorbitan ester surfactant in combination with an octoxynol or a polyoxyethylene alkyl ether or ester surfactant in combination with at least one additional non-ionic surfactant such as an octoxynol; (P) an immuno-stimulatory oligonucleic acid (e.g. a CpG oligonucleic acid) and a saponin; (O) an immuno-stimulant and a particle of metal salt; (R) a saponin and an oil-in-water emulsion; (S) a saponin (e.g. QS21)+3dMPL+IL12 (optionally+a sterol); (T) E. coli heat-labile enterotoxin (“LT”), or detoxified mutants thereof, such as the K63 or R72 mutants; (U) cholera toxin (“CT”), or diphtheria toxin (“DT”) or detoxified mutants of either; (V) double-stranded RNA; (W) monophosphoryl lipid A mimics, such as aminoalkyl glucosaminide phosphate derivatives e.g. RC-529]; (X) polyphosphazene (PCPP); or (Y) a bioadhesive such as esterified hyaluronic acid microspheres or a mucoadhesive such as crosslinked derivatives of poly(acrylic acid), polyvinyl alcohol, polyvinyl pyrollidone, polysaccharides and carboxymethylcellulose.

In some embodiments, administration of the antibody or antigen-binding fragment thereof is intended to reduce the severity of the pathologic condition. By the term “reduce the severity of the pathologic condition,” it is to be understood that any reduction via the methods, compounds, and compositions disclosed herein is to be considered encompassed by the invention. In some embodiments, reduction in severity may comprise enhancement of survival, or in another embodiment, halting disease progression, or in another embodiment, delay in disease progression.

In one embodiment, dosing is dependent on the cellular responsiveness to the administered molecules/compounds or compositions comprising same. In general, the doses utilized for the above described purposes will vary, but will be in an effective amount to exert the desired effect, as determined by a clinician of skill in the art. As used herein, the term “pharmaceutically effective amount” refers to an amount of a compound as described herein, which will produce the desired alleviation in symptoms or other desired phenotype in a patient.

In one embodiment, the concentrations of the antibody or antigen-binding fragment thereof will depend on various factors, including the nature of the condition to be treated, the condition of the patient, the route of administration, and the individual tolerability of the compositions.

In some embodiments, any of the compositions of this invention will consist essentially of the antibody or antigen-binding fragment thereof as described herein. In some embodiments, the term “comprise” refers to the inclusion of the indicated active agent, such as the antibody or antigen-binding fragment thereof of this invention, as well as inclusion of other active agents, and pharmaceutically acceptable carriers, excipients, emollients, stabilizers, etc., as are known in the pharmaceutical industry.

In some embodiments, the compositions of this invention will consist essentially of a polypeptide/polynucleic acid/vector as herein described. In some embodiments, the term “consisting essentially of” refers to a composition whose only active ingredient of a particular class of agents, is the indicated active ingredient, however, other compounds may be included which are involved directly in the therapeutic effect of the indicated active ingredient. In some embodiments, the term “consisting essentially of” refers to a composition whose only active ingredient of targeting a particular mechanism, or acting via a particular pathway, is the indicated active ingredient, however, other compounds may be included which are involved directly in the therapeutic effect of the indicated active ingredient, which for example have a mechanism of action related to but not directly to that of the indicated agent. In some embodiments, the term “consisting essentially of” refers to a composition whose only active ingredient is the indicated active ingredient. However, other compounds may be included which are for stabilizing, preserving, etc., the formulation, but are not involved directly in the therapeutic effect of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

It will be appreciated that the actual amounts of the antibody or antigen-binding fragment in a specific case will vary according to the particular compositions formulated, the mode of application, and the particular conditions and organism being treated. Dosages for a given host can be determined using conventional considerations, e.g., by customary comparison of the differential activities of the subject compounds and of a known agent, e.g., by means of an appropriate, conventional pharmacological protocol.

In one embodiment, the compounds of the invention are administered acutely for acute treatment of temporary conditions, or may be administered chronically, especially in the case of progressive, recurrent, or degenerative disease. In one embodiment, one or more compounds of the invention may be administered simultaneously, or in another embodiment, they may be administered in a staggered fashion. In one embodiment, the staggered fashion may be dictated by the stage or phase of the disease.

Parenteral vehicles (for subcutaneous, intravenous, intraarterial, or intramuscular injection) include sodium chloride solution, Ringer's dextrose, dextrose, sodium chloride, lactated Ringer's, and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose, and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In one embodiment, the route of administration may be parenteral, or a combination thereof. In another embodiment, the route may be intra-ocular, conjunctival, topical, transdermal, intradermal, subcutaneous, intraperitoneal, intravenous, intra-arterial, vaginal, rectal, intratumoral, parcanceral, transmucosal, intramuscular, intravascular, intraventricular, intracranial, inhalation (aerosol), nasal aspiration (spray), intranasal (drops), sublingual, oral, aerosol or suppository or a combination thereof. In one embodiment, the dosage regimen will be determined by skilled clinicians, based on factors such as exact nature of the condition being treated, the severity of the condition, the age and general physical condition of the patient, body weight, and response of the individual patient.

For intranasal administration or application by inhalation, solutions or suspensions of the compounds mixed and aerosolized or nebulized in the presence of the appropriate carrier suitable. Such an aerosol may comprise any agent described herein.

For parenteral application, particularly suitable are injectable, sterile solutions, preferably oily or aqueous solutions, as well as suspensions, emulsions, or implants, including suppositories and enemas. Ampoules are convenient unit dosages. Such a suppository may comprise any agent described herein.

Sustained or directed release compositions can be formulated, e.g., liposomes or those wherein the antibody or antigen-binding fragment is protected with differentially degradable coatings, e.g., by microencapsulation, multiple coatings, etc. Such compositions may be formulated for immediate or slow release. It is also possible to freeze-dry the new compounds and use the lyophilisates obtained, for example, for the preparation of products for injection.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate.

Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

In one embodiment, a composition of or used in the methods of this invention may be administered alone or within a composition. In another embodiment, compositions of this invention admixture with conventional excipients, i.e., pharmaceutically acceptable organic or inorganic carrier substances suitable for parenteral, enteral (e.g., oral) or topical application which do not deleteriously react with the active compounds may be used. In one embodiment, suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides, and diglycerides, pentaerythritol fatty acid esters, hydroxy methylcellulose, polyvinyl pyrrolidone, etc. In another embodiment, the pharmaceutical preparations can be sterilized and, if desired, mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. In another embodiment, they can also be combined where desired with other active agents, e.g., vitamins.

Pharmaceutical compositions include “pharmaceutically acceptable” and “physiologically acceptable” carriers, diluents or excipients. In one embodiment, the terms “pharmaceutically acceptable” and “physiologically acceptable” refers to any formulation which is safe, and provides the appropriate delivery for the desired route of administration of an effective amount of at least one antibody or antigen-binding fragment for use in the present invention. This term refers to the use of buffered formulations as well, wherein the pH is maintained at a particular desired value, ranging from pH 4.0 to pH 9.0, in accordance with the stability of the compounds and route of administration. The terms include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary active compounds (e.g., preservatives, antibacterial, antiviral and antifungal agents) can also be incorporated into the compositions.

Pharmaceutical compositions of the present invention can include one or more further chemotherapeutic agents selected from the group consisting of nitrogen mustards (e.g., cyclophosphamide and ifosfamide), aziridines (e.g., thiotepa), alkyl sulfonates (e.g., busulfan), nitrosoureas (e.g., carmustine and streptozocin), platinum complexes (e.g., carboplatin and cisplatin), non-classical alkylating agents (e.g., dacarbazine and temozolamide), folate analogs (e.g., methotrexate), purine analogs (e.g., fludarabine and mercaptopurine), adenosine analogs (e.g., cladribine and pentostatin), pyrimidine analogs (e.g., fluorouracil (alone or in combination with leucovorin) and gemcitabine), substituted ureas (e.g., hydroxyurea), antitumor antibiotics (e.g., bleomycin and doxorubicin), epipodophyllotoxins (e.g., etoposide and teniposide), microtubule agents (e.g., docetaxel and paclitaxel), camptothecin analogs (e.g., irinotecan and topotecan), enzymes (e.g., asparaginase), cytokines (e.g., interleukin-2 and interferon-α), monoclonal antibodies (e.g., trastuzumab and bevacizumab), recombinant toxins and immunotoxins (e.g., recombinant cholera toxin-B and TP-38), cancer gene therapies, physical therapies (e.g., hyperthermia, radiation therapy, and surgery) and cancer vaccines (e.g., vaccine against telomerase).

The compositions (e.g., antibodies, bispecific molecules) of the invention can also be administered together with complement. Accordingly, within the scope of the invention are compositions comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules of the invention and the complement or serum can be administered separately.

Pharmaceutical compositions can be formulated to be compatible with a particular local or systemic route of administration. Thus, pharmaceutical compositions include carriers, diluents, or excipients suitable for administration by particular routes. Specific non-limiting examples of routes of administration for compositions of the invention are inhalation or intranasal delivery. Additional routes include parenteral, e.g., intravenous, intradermal, subcutaneous, oral, transdermal (topical), transmucosal, and rectal administration.

Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate-buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion, and by the use of surfactants. Antibacterial and antifungal agents include, for example, parabens, chlorobutanol, phenol, ascorbic acid, and thimerosal. Isotonic agents, for example, sugars, polyalcohols such as manitol, sorbitol, sodium chloride can be included in the composition. Including an agent that delays absorption, for example, aluminum monostearate and gelatin can prolong absorption of injectable compositions.

Sterile injectable solutions can be prepared by incorporating the antibody or antigen-binding fragment thereof in the required amount in an appropriate solvent with one or a combination of the above ingredients followed by filtered sterilization. Generally, dispersions are prepared by incorporating the antibody or antigen-binding fragment thereof into a sterile vehicle containing a basic dispersion medium and other ingredients as above. In the case of sterile powders for the preparation of sterile injectable solutions, methods of preparation include, for example, vacuum drying and freeze-drying which yields a powder of the active ingredient plus any additional desired ingredient from a previously sterile-filtered solution thereof.

For transmucosal or transdermal administration, penetrants appropriate to the barrier be permeated are used in the formulation. Such penetrants are generally known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, or creams, as generally known in the art.

The present invention's antibodies, including subsequences and modified forms and nucleic acids encoding them, can be prepared with carriers that protect against rapid elimination from the body, such as a controlled release formulation or a time delay material such as glyceryl monostearate or glyceryl stearate. The compositions can also be delivered using implants and microencapsulated delivery systems to achieve local or systemic sustained delivery or controlled release.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to cells or tissues using antibodies or viral coat proteins) can also be used as pharmaceutically acceptable carriers. These can be prepared according to methods known to those skilled in the art, for example, as described in U.S. Pat. No. 4,522,811.

Additional pharmaceutical formulations appropriate for the compositions for administration in the methods of the invention are known in the art (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993)). The pharmaceutical formulations can be packaged in dosage unit form for ease of administration and uniformity of dosage. “Dosage unit form” as used herein refers to physically discrete units suited as unitary dosages for the subject to be treated; each unit containing a predetermined quantity of the antibody or antigen-binding fragment thereof calculated to produce the desired therapeutic effect in association with the pharmaceutical carrier or excipient.

Although the pharmaceutical compositions provided herein are principally directed to pharmaceutical compositions which are suitable for administration to humans, it will be understood by the skilled artisan that such compositions are generally suitable for administration to animals of all sorts. Modification of pharmaceutical composition suitable for administration to humans in order to render the compositions suitable for administration to various animals is well understood, and the ordinarily skilled veterinary pharmacologist can design and perform such modification with little, if any, experimentation. Subjects to which administration of the pharmaceutical compositions of the invention is contemplated include, but are not limited to, humans and other primates, and other mammals.

It is to be understood that any amino acid sequence, whether obtained naturally or synthetically by any means, exhibiting sequence, structural or functional homology to the polypeptides described herein, are considered part of this invention.

It is to be understood that reference to any publication, patent application or issued patent is to be considered as fully incorporated herein by reference in its entirety.

It is to be understood that any assay for measuring a particular activity which is modulated by the therapeutic compound may be employed, as a means of determining the efficacy of the compound, in one embodiment, optimal loading of the compound, in another embodiment, timing, and dosage, in another embodiment, or a combination thereof.

### Methods of Detecting PSMA

It is to be understood by a skilled artisan that the antibody, antigen-binding fragments, or compositions provided herein can be used in diagnostic or therapeutic procedures.

In one embodiment, provided herein is a method of diagnosing the presence of a tumor or cancer growth in a subject. In another embodiment, the method comprises sampling a tissue sample isolated from the subject with a composition comprising the antibody or antigen-binding fragment provided herein, whereby specific binding of the antibody or antigen-binding fragment to the tissue sample is indicative of the presence of a tumor or cancer growth in the subject.

In another embodiment, the method further comprises detecting a secondary reagent that specifically binds to the antibody or antigen-binding fragment but does not antagonize binding of the antibody or antigen-binding fragment to its target. In another embodiment, the “secondary reagent” is a photoactivatable agent, an enzyme, a tag, a label, a fluorophore, a radioisotope, a bioluminescent protein, a bioluminescent peptide, a fluorescent tag, a fluorescent protein, or a fluorescent peptide.

In one embodiment, the term “cancer” and “cancerous” refer to or describe, in one embodiment, the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwannoma, meningioma, adenocarcinoma, melanoma, and leukemia or lymphoid malignancies.

In one embodiment, the term “cancer” includes but is not limited to, ovarian cancers, breast cancers, glioblastoma, gastrointestinal cancers. In another embodiment, the cancer is prostate cancer.

In another embodiment, “sampling” comprises the step of testing or analyzing the sample using a detection assay that enables the detection of a secondary reagent that is complexed with or conjugated to the antibody or antigen-binding fragment and emits a detectable “signal” when the antibody or antigen-binding fragment is specifically bound to the target. In another embodiment, the detection is achieved using assays routinely used in the art, such as, but not limited to, immunological assays (e.g., immunohistochemistry, ELISA, etc.) or microscopic imaging.

In one embodiment, the term “labeled” refers to antibodies of the invention having one or more elements, isotopes, or chemical compounds attached to enable the detection in a screen. In general, labels fall into three classes: a) immune labels, which may be an epitope incorporated as a fusion partner that is recognized by an antibody, b) isotopic labels, which may be radioactive or heavy isotopes, and c) small molecule labels, which may include fluorescent and calorimetric dyes or molecules such as biotin that enable other labeling methods. In one embodiment, antibodies of the invention are labeled with biotin. In other related embodiments, biotinylated antibodies of the invention may be used, for example, as an imaging agent or as a means of identifying one or more ligand molecules. In another embodiment, the label can be a nanoparticle that can be detected or visualized once bound to the antibody or antigen-binding fragment. Labels may be incorporated into the compound at any position and may be incorporated in vitro or in vivo during protein expression.

In one embodiment, the conjugate formed by the antibody or antigen-binding fragment and the secondary reagent provided herein are used for various applications such as, but not limited to, flow cytometry, ELISA, Western blotting, immunohistochemistry, membrane assays, and diagnostic and therapeutic methods as further described herein or as routinely applied in the art.

Imaging of Tumors with Abnormal PSMA Expression

In one embodiment, the composition of the present invention is administered to a subject having a disease involving inappropriate expression of a target antigen, a protein or other molecule. For example, in one embodiment, the composition comprising an antibody or antigen-binding fragment thereof that binds to PSMA is administered to detect the presence, abundance, location, or combination thereof of PSMA in the subject. Within the scope of this disclosure are diseases and disorders characterized by aberrant proteins, due, for example, to alterations in the amount of a protein present, protein localization, posttranslational modification, conformational state, the presence of a mutant or pathogen protein, etc. Similarly, the disease or disorder may be characterized by alterations in molecules, including but not limited to polysaccharides and gangliosides. An overabundance may be due to any cause, including but not limited to overexpression at the molecular level, prolonged or accumulated appearance at the site of action, or increased activity of a protein relative to normal. Included within this definition are diseases and disorders characterized by a reduction of a protein. This reduction may be due to any cause, including but not limited to reduced expression at the molecular level, shortened or reduced appearance at the site of action, mutant forms of a protein, or decreased activity of a protein relative to normal. Such an overabundance or reduction of a protein can be measured relative to normal expression, appearance, or activity of a protein, and the measurement may play an important role in the development and/or clinical testing of the antibodies of the present invention.

In one embodiment, the disclosed antibody or antigen-binding fragment thereof binds the antigen expressed on tumor cells, such as prostate cancer cells when administrated in a subject. In another embodiment, the antibody or antigen-binding fragment thereof administrated in a subject binds the antigen expressed on neovasculature of solid tumors, such as the tumors with PSMA positive neovasculature, including but not limited to lung cancer, liver cancer, pancreas cancer, colon cancer, gastric cancer, breast cancer, ovarian cancer, kidney cancer, prostate cancer, bladder cancer, melanoma, glioma etc.

In one embodiment, provided herein is a method of imaging a PSMA-containing tumor. In some embodiments, the method comprises administering to a subject the antibody or antigen-binding fragment thereof described herein or contacting a tissue sample with the antibody or antigen-binding fragment thereof. In another embodiment, the prostate or other types solid tumor can be visualized once the antibody or antigen-binding fragment has bound its target, e.g., PSMA-containing tissue or organ. In some embodiments, the antibody or antigen-binding fragment thereof is operably linked to a secondary reagent. In yet another embodiment, the secondary reagent comprises a photoactivatable agent, an enzyme, a tag, a label, a fluorophore, a radioisotope, a bioluminescent protein, a bioluminescent peptide, a fluorescent tag, a fluorescent protein, or a fluorescent peptide. Non-limiting examples of secondary reagents are provided below.

In one embodiment, the detectable label or secondary reagent attached thereto, include labels such as, but not limited to, a fluorescent label (e.g., fluorescein, isothiocyanate (FITC), a cyanine dye, etc.), an affinity label (e.g., biotin, avidin, protein A, etc.), an enzymatic label (e.g., horseradish peroxidase or alkaline phosphatase), or an isotopic label (e.g., 1241) or any other such detectable moiety to allow for detection and isolation of the antibody.

Detection methods for identification of binding species within the population of altered variable regions can be direct or indirect and can include, for example, the measurement of light emission, radioisotopes, calorimetric dyes, and fluorochromes. Direct detection includes methods that operate without intermediates or secondary measuring procedures to assess the amount of bound antigen or ligand. Such methods generally employ ligands that are themselves labeled by, for example, radioactive, light emitting or fluorescent moieties. In contrast, indirect detection includes methods that operate through an intermediate or secondary measuring procedure. These methods generally employ molecules that specifically react with the antigen or ligand and can themselves be directly labeled or detected by a secondary reagent. For example, an antibody specific for a ligand can be detected using a secondary antibody capable of interacting with the first antibody specific for the ligand, again using the detection methods described above for direct detection. Indirect methods can additionally employ detection by enzymatic labels. Moreover, for the specific example of screening for catalytic antibodies, the disappearance of a substrate or the appearance of a product can be used as an indirect measure of binding affinity or catalytic activity.

In some embodiments, the antibody or antigen-binding fragment thereof is labeled with near-infrared dye. For example, the antibody or antigen-binding fragment thereof may be labeled with IRDye800CW or Indocyanine Green (ICG).

In some embodiments, the antibody or antigen-binding fragment thereof is attached to macrocyclic chelators useful for conjugating radiometal ions, including but not limited to, 111In, 177Lu, 90Y, 166Ho, 153Sm, 215Bi, and 225Ac to polypeptides. In some embodiments, the radiometal ion associated with the macrocyclic chelators attached to antibodies of the invention is 111In. In some embodiments, the radiometal ion associated with the macrocyclic chelator attached to antibodies polypeptides of the invention is 90Y. In specific embodiments, the macrocyclic chelator is 1,4,7,10-tetraazacyclododecane-N,N′,N″,N″′-tetraacetic acid (DOTA). In specific embodiments, the macrocyclic chelator is .quadrature.-(5-isothiocyanato-2-methoxyphenyl)-1,4,7,10-tetraaza-cyclodo-decane-1,4,7,10-tetraacetic acid. In other specific embodiments, the DOTA is attached to the antibody of the invention via a linker molecule. Examples of linker molecules useful for conjugating a macrocyclic chelator such as DOTA to a polypeptide are commonly known in the art; see, for example, DeNardo et al., Clin Cancer Res. 4(10):2483-90, 1998; Peterson et al., Bioconjug. Chem. 10(4):553-7, 1999; and Zimmerman et al., Nucl. Med. Biol. 26(8):943-50, 1999, which are hereby incorporated by reference in their entirety. In addition, U.S. Pat. Nos. 5,652,361 and 5,756,065, which disclose chelating agents that may be conjugated to antibodies, and methods for making and using them, are hereby incorporated by reference in their entireties.

### Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, or at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity or at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wildtype protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

The term “Kassoc” or “Ka,” as used herein, is intended to refer to the association rate of a particular antibody-antigen interaction, whereas the term “Kdis” or “Kd,” as used herein, is intended to refer to the dissociation rate of a particular antibody-antigen interaction. The term “KD,” as used herein, is intended to refer to the dissociation constant, which is obtained from the ratio of Kd to Ka (i.e., Kd/Ka) and is expressed as a molar concentration (M). KD values for antibodies can be determined using methods well established in the art. As an example, a method for determining the KD of an antibody is by using surface plasmon resonance, preferably using a biosensor system such as a BIACORE system.

The term “detectable label” as used herein refers to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens), intercalating dyes and the like. The term “fluorescer” refers to a substance or a portion thereof that is capable of exhibiting fluorescence in the detectable range.

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of disorders (e.g., neuronal disorders, autoimmune diseases, and cardiovascular diseases), and/or who has been diagnosed with inflammatory disorders. In some embodiments, the subject is a mammal. In some embodiments, the subject is human.

As used herein, the term “disease” is intended to be generally synonymous and is used interchangeably with the terms “disorder” and “condition” (as in medical condition), in that all reflect an abnormal condition (e.g., inflammatory disorder, cancer) of the human or animal body or of one of its parts that impairs normal functioning, is typically manifested by distinguishing signs and symptoms, and causes the human or animal to have a reduced duration or quality of life.

The terms “decrease,” “reduced,” “reduction,” “decrease,” or “inhibit” are all used herein generally to mean a decrease by a statistically significant amount. However, for avoidance of doubt, “reduced,” “reduction,” “decrease,” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “agent” denotes a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The term “therapeutic effect” is art-recognized and refers to a local or systemic effect in animals, particularly mammals, and more particularly humans caused by a pharmacologically active substance.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit]“per kg (or g, mg etc.) bodyweight,” even if the term “bodyweight” is not explicitly mentioned.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest, such as antibodies. The sample may be any tissue sample from the subject. The sample may comprise protein from the subject.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition that is “substantially free” from Y may be completely free from Y. Where necessary, the word “substantially” may be omitted from the definition of the invention.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15, 14%, 13%, 12%, 1%, 1%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

Engineering of the N=>S mutation in CDR3 region of the light chain of PSMA antibodies There is a high potential of deamidation of asparagine in the CDR3 of a light chain of PSMAb appearing as the hot spot of NG. To improve the stability of PSMAb by abrogating the potential deamidation, the NG motif need to be removed. The CDR3 region is the most important binding site with antigen, therefore, mutation of the amino acids in this region usually leads to significant affinity and function decline. We tried to remove NG motif by mutating asparagine to serine and examined the function of this mutated antibody. The mutation was introduced using overlapping PCR to mutate the asparagine codon AAT to serine codon AGT. The CDR3 region of PSMAb before mutation was: GCA ACA TGG GAT GAC AGT CTG AAT GGT GTA ATA (SEQ ID NO: 80); while the mutated CDR3 in PSMAbLm was: GCA ACA TGG GAT GAC AGT CTG AGT GGT GTA ATA (SEQ ID NO: 81). The encoding CDR3 region was therefore mutated to ATWDDSLSGVI (SEQ ID NO: 17) in PSMAbLm from the original ATWDDSLNGVI (SEQ ID NO: 82) in PSMAb.

### Example 2

### Preparation of Afucosylated Full Antibody of PSMAbLm and PSMAb

Heavy and light chain genes for PSMAbLm and PSMAb were cloned into a bicistronic full antibody expression vector, respectively, for expression.

Wild type PSMAb and PSMAbLm that have normal fucosylation modification were expressed in regular CHO-K1 cells, while the afucosylated PSMAb and PSMAbLm were expressed in Fut8−/− CHO-K1, which is a genome-modified CHO-K1 cell line that was 100% abolished in fucose synthesis. Afucosylated PSMAbLm and PSMAb antibodies were named as PSMAbLmf and PSMAbf.

Fut8−/− CHO cells were cultured in DMEM+5% FBS, supplemented with Penicillin/Streptomycin. CHO cells were harvested from a tissue culture plate by trypsinization and electroporated with the PSMAbLm and PSMAb expression plasmid. After electroporation, cells were cultured in DMEM+5% FBS and selected with Zeocin for 14 days, and stable cell colonies appeared, which were harvested as stable CHO cell pools to express afucosylated antibodies. Stable cell pools were further adapted to grow in serum-free medium and suspension culture for convenient purification of recombinant antibodies.

Stable cell pools were cultured for 7-14 days to express antibodies, followed by purification from the supernatant using Protein A chromatography. Purified antibodies were buffer exchanged in PBS, aliquoted, and stored in −80° C.

### Example 3

### Affinity Measurement of PSMAbLm AND PSMAb by Capture ELISA

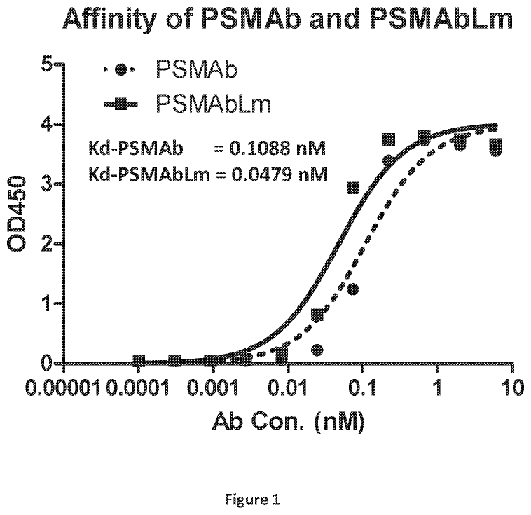

Goat anti-human-IgG1 (Fc specific) (Jackson ImmunoResearch Laboratories, Inc, Pennsylvania, USA) was coated on wells of ELISA plate in 100 μl PBS at the concentration of 3 μg/ml, 4° C. overnight. The wells were washed twice with 200 μl PBST (PBS, pH7.4, containing 0.05% Tween 20) and then blocked with 400 μl PBS™ (PBST containing 5% dry milk) for 2 h at room temperature. Three-fold serially diluted PSMAb and PSMAbLm starting from 6 nM down to 0.1 pM in 50 μl PBS™ were then incubated with the wells respectively for 1 h at room temperature. After 6 washes with PBST, the wells were incubated with 50 μl biotinylated PSMA extracellular domain recombinant protein (Acrobiosystems, Delaware, USA) at the concentration of 0.5 μg/ml in PBS™ for 1 h at room temperature. After incubation, the wells were washed 6 times using PBST and incubated with 50 μl 1:1000 diluted streptavidin-HRP (BD bioscience, California, USA) in PBS™. After final 6 washes with PBST, colorimetric signals were developed by incubation with 70 μl 3, 3′, 5, 5′-tetramethylbenzidine (TMB, eBioscience, CA, USA) and for 20 min at room temperature, then stopped by incubation with 70 μl 1 M H₂SO₄. The absorbance was measured at 450 nm using a microplate reader (VWR, PA, USA). The binding curves were analyzed using GraphPad Prism 5.0 software. The ELISA results are shown in Table 1 and FIG. 1.

TABLE 1
OD450 readings of PSMAb and PSMAbLm at different concentrations

| Ab Con. (nM) | 6 | 2 | 0.6667 | 0.2222 | 0.0741 | 0.0247 | 0.0082 | 0.0027 | 0.0009 | 0.0003 | 0.0001 | 0 |
|---|---|---|---|---|---|---|---|---|---|---|---|---|
| PSMAb | 3.578 | 3.674 | 3.798 | 3.441 | 1.247 | 0.219 | 0.069 | 0.047 | 0.046 | 0.046 | 0.045 | 0.045 |
| PSMAb | 3.542 | 3.644 | 3.655 | 3.342 | 1.243 | 0.231 | 0.068 | 0.048 | 0.047 | 0.046 | 0.045 | 0.046 |
| PSMAb | 3.549 | 3.629 | 3.738 | 3.39 | 1.244 | 0.228 | 0.063 | 0.046 | 0.047 | 0.045 | 0.046 | 0.045 |
| PSMAbLm | 3.541 | 3.742 | 3.767 | 3.722 | 2.922 | 0.792 | 0.173 | 0.068 | 0.05 | 0.045 | 0.045 | 0.044 |
| PSMAbLm | 3.663 | 3.662 | 3.819 | 3.751 | 2.941 | 0.827 | 0.181 | 0.067 | 0.048 | 0.047 | 0.044 | 0.045 |
| PSMAbLm | 3.788 | 3.828 | 3.838 | 3.765 | 2.94 | 0.831 | 0.178 | 0.067 | 0.05 | 0.047 | 0.045 | 0.045 |

Surprisingly, data showed that PSMAbLm has an about 2.3-fold higher affinity than its parent antibody PSMAb, i.e., Kd=0.0479 nM (PSMAbLm) vs Kd=0.1088 nM (PSMAb) (FIG. 1). CDR3 regions of both heavy and light chains are pivotal for antigen binding. Mutation in these regions usually leads to dramatic affinity loss of an antibody. However, single mutation from N to S in the light chain CDR3 unexpectedly increased the affinity by about 2.3 folds, which is very rare, especially when the affinity of PMSAb is already very high, almost reaching the roof of an antibody affinity in the field and leaving nearly no room for further improvement.

### Example 4

### ADCC Assay of PSMAb and PSMAbLm on PSMA+CHO Cells

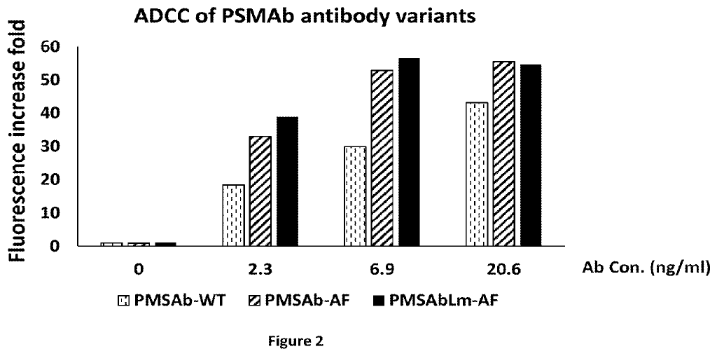

ADCC is one of the most important mechanism of an antibody to kill target cells, such as tumor killing. To improve ADCC activity, afucosylated PSMAb and PSMAbLm were prepared and their functions were investigated. One day before the assay, target cells (PMSA+CHO cells) were seeded at 10,000 cells/well into Falcon white opaque 96-well plate (Cat. #353 296) in 100 μL medium and cultured at 37° C. for overnight. On the next day, 75-μL medium was removed from each well, and 200,000 CD16-Jurkat reporter cells/25 μL/well plus 25 μL of antibodies with 3-fold serial dilutions (from 20.6 pg/mL highest concentration) were added, and the cell mixtures were cultured at 37° C. for 6 h. At the end of the culture, the plates were cooled at room temperature for 15 min, and 60 μL Bio-Glo luciferase assay buffer (Promega, Cat. #G719A) was added to each well. Luciferase signals were read on a fluorometer within 5-30 min. Signal increase fold comparing to the negative control well was calculated to reflect the ADCC activity. Results are shown in Table 2 and FIG. 2.

TABLE 2
Luciferase signal increase fold of PSMAb-WT, PSMAb-AF
and PSMAbLm-AF at different concentrations

| Ab Con. (pg/ml) | 0 | 2.286 | 6.859 | 20.576 |
|---|---|---|---|---|
| PMSAb-WT | 1.038685 | 18.41199 | 29.98259 | 43.10832 |
| PMSAb-AF | 0.858801 | 33.03482 | 52.9323 | 55.54932 |
| PMSAbLm-AF | 1.102515 | 38.94778 | 56.58801 | 54.73114 |

Data showed that PSMAb with normal fucosylation modification (PSMAb-WT) had relatively lower ADCC activity, while afucosylated PSMAb had a higher ADCC function. Comparing to PSMAb, PSMAbLm showed a similar, if not a higher ADCC activity (FIG. 2).

### Example 5

### Binding of PSMAb and PSMAbLm on LnCap Cells

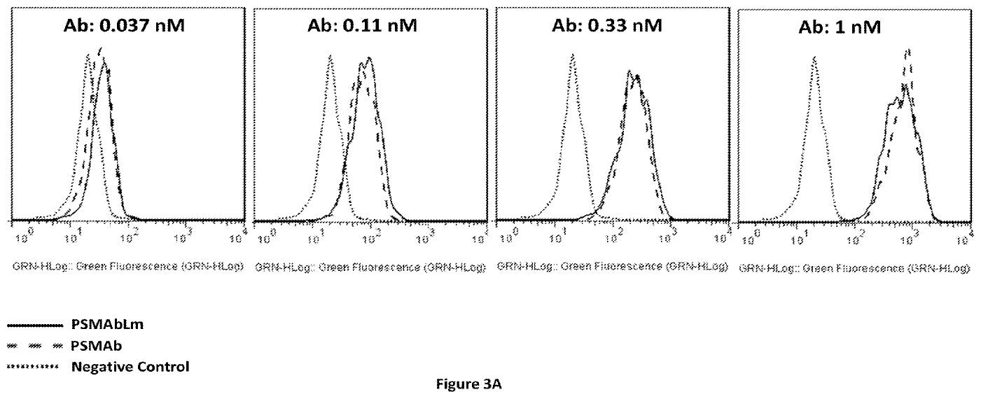

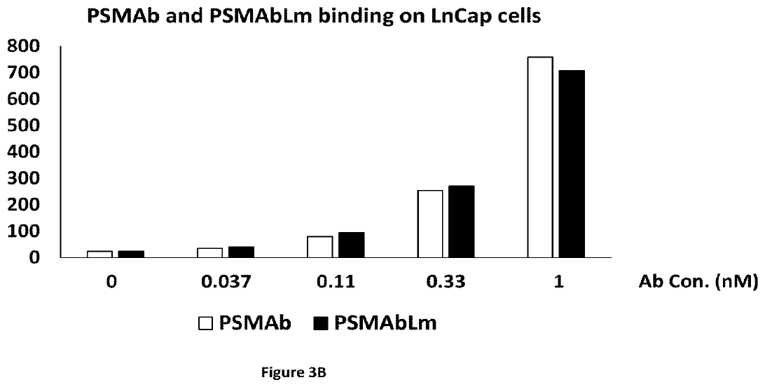

PSMAb and PSMAbLm were labeled with Alexa488 by incubation with 2-fold higher concentration of anti-human-IgG-Alexa488 (Jackson ImmunoResearch Laboratories, Inc, Pennsylvania, USA) at room temperature for 2 hours. LnCap cells cultured in RPMI 1640 containing 10% FBS were detached with Versene buffer, washed twice with 15 ml PBS and incubated with 3-fold serially diluted Alexa488 labeled PSMAb and PSMAbLm respectively in medium (RPMI 1640 containing 10% FBS) on ice for 1 hour. After twice washed with cold medium, antibody bindings on cells were analyzed by flowcytometry. PSMAb and PSMAbLm showed a similar binding patterns on LnCap cells at each concentration (Table 3, FIG. 3A and FIG. 3B).

TABLE 3
Mean fluorescence intensity of cells stained
by different antibody concentrations

| Ab Con. (nM) | PSMAb | PSMAbLm |
|---|---|---|
| 0 | 23 | 22.7 |
| 0.037 | 35.4 | 40.3 |
| 0.11 | 80.3 | 94.2 |
| 0.33 | 255 | 270 |
| 1 | 759 | 708 |

### Example 6

Internalization of PSMAb and PSMAbLm into LnCap Cells

Internalization is the prerequisite of an antibody to deliver toxic drugs into cells, which is the mechanism of antibody drug conjugate (ADC) for tumor therapy. To evaluate the potential of PMSAbLm for ADC development, its internalization efficiency was investigated. One day before assay, LnCap cells were seeded in a 96 well plate, 20000 cells/well in 200 μL RPMI 1640 containing 10% FBS. The next day, the medium were removed and the cells were incubated with 3-fold serially diluted Alexa488 labeled PSMAb and PSMAbLm respectively at 37° C. in tissue culture incubator. Labeling of the antibody with Alexa488 was described in EXAMPLE 5. After 2 hours incubation, the cells were washed twice by PBS and 50 μL trypsin was added to detach cells, meanwhile to remove any antibodies still bound to cell membrane that were not internalized. The cells were washed twice with medium and the internalization of antibodies were analyzed by flowcytometry.

TABLE 4
Mean fluorescence Intensity of LnCap cells incubated
with different antibody concentrations

| Ab Con. (nM) | 0 | 0.001 | 0.003 | 0.009 | 0.027 | 0.082 | 0.247 | 0.74 | 2.22 | 6.67 |
|---|---|---|---|---|---|---|---|---|---|---|
| PSMAb | 21.8 | 22.5 | 23.3 | 27.4 | 39.6 | 81.4 | 190 | 587 | 1182 | 2447 |
| PSMAbLm | 21.4 | 21.4 | 23.8 | 29.1 | 51.4 | 106 | 287 | 750 | 1700 | 2711 |

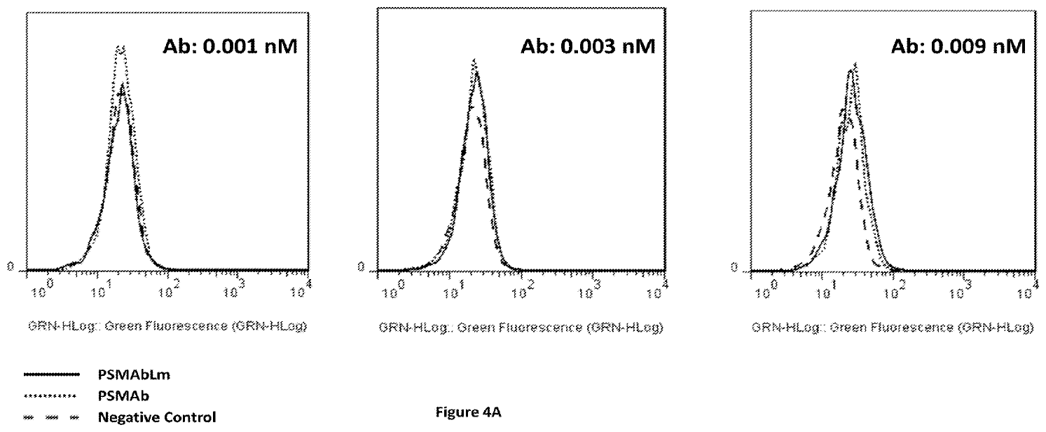

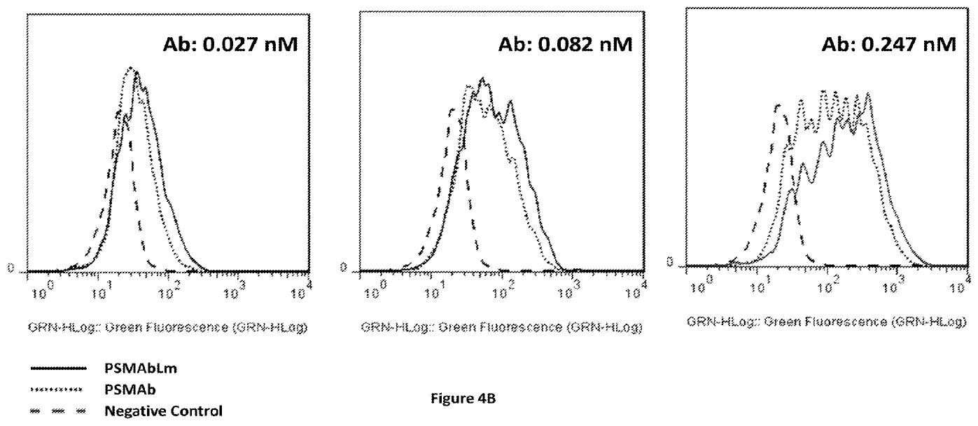

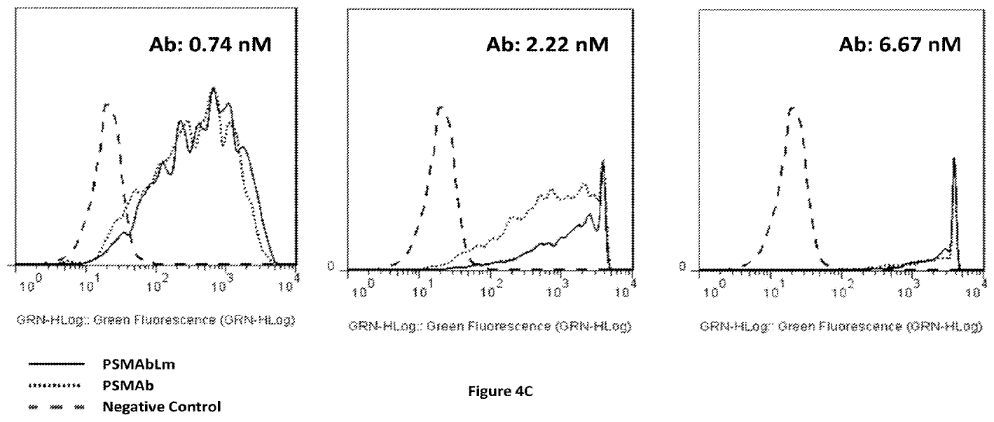

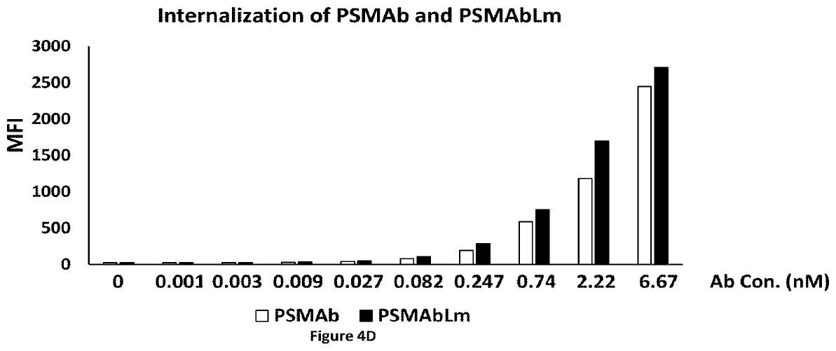

The results showed that both PSMAb and PSMAbLm could be internalized highly efficiently, with the lowest concentration allowing for obvious internalization at 0.027 nM for both antibodies. Surprisingly, PSMAbLm showed a higher internalization efficiency than PSMAb (Table 4 and FIG. 4A-4D), indicating PMSAbLm is better than PSMAb for ADC development.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An antibody or antigen-binding fragment thereof or a variant thereof that specifically binds to prostate specific membrane antigen (PSMA), wherein the antibody or antigen-binding fragment thereof comprises:
a light chain variable region (LCVR) comprising three light chain complementarity determining regions (LCDRs) (LCDR1, LCDR2, and LCDR3), wherein the LCDR1 comprises the amino acid sequence of SEQ ID NO: 9, the LCDR2 comprises the amino acid sequence of SEQ ID NO: 13 or 41, and the LCDR3 comprises the amino acid sequence of SEQ ID NO: 17; and/or
a heavy chain variable region (HCVR) three heavy chain complementarity determining regions (HCDRs) (HCDR1, HCDR2, and HCDR3), comprising wherein the HCDR1 comprises the amino acid sequence of SEQ ID NO: 25 or 45, the HCDR2 comprises the amino acid sequence of SEQ ID NO: 29, and the HCDR3 comprises the amino acid sequence of SEQ ID NO: 33 or 49,
wherein the variant comprises one or more conservative amino acid substitutions in the LCDR1, the LCDR2, the LCDR3, the HCDR1, the HCDR2, or the HCDR3.

2. The antibody or antigen-binding fragment thereof or variant thereof of claim 1, wherein:
(a) the LCDR1 comprises the amino acid sequence of SEQ ID NO: 9, the LCDR2 comprises the amino acid sequence of SEQ ID NO: 13, the LCDR3 comprises the amino acid sequence of SEQ ID NO: 17; the HCDR1 comprises the amino acid sequence of SEQ ID NO: 25, the HCDR2 comprises the amino acid sequence of SEQ ID NO: 29, and the HCDR3 comprises the amino acid sequence of SEQ ID NO: 33;
(b) the LCDR1 comprises the amino acid sequence of SEQ ID NO: 9, the LCDR2 comprises the amino acid sequence of SEQ ID NO: 13, the LCDR3 comprises the amino acid sequence of SEQ ID NO: 17; the HCDR1 comprises the amino acid sequence of SEQ ID NO: 45, the HCDR2 comprises the amino acid sequence of SEQ ID NO: 29, and the HCDR3 comprises the amino acid sequence of SEQ ID NO: 33;
(c) the LCDR1 comprises the amino acid sequence of SEQ ID NO: 9, the LCDR2 comprises the amino acid sequence of SEQ ID NO: 41, the LCDR3 comprises the amino acid sequence of SEQ ID NO: 17; the HCDR1 comprises the amino acid sequence of SEQ ID NO: 45, the HCDR2 comprises the amino acid sequence of SEQ ID NO: 29, and the HCDR3 comprises the amino acid sequence of SEQ ID NO: 33; or
(d) the LCDR1 comprises the amino acid sequence of SEQ ID NO: 9, the LCDR2 comprises the amino acid sequence of SEQ ID NO: 13, the LCDR3 comprises the amino acid sequence of SEQ ID NO: 17; the HCDR1 comprises the amino acid sequence of SEQ ID NO: 45, the HCDR2 comprises the amino acid sequence of SEQ ID NO: 29, and the HCDR3 comprises the amino acid sequence of SEQ ID NO: 49.

3. The antibody or antigen-binding fragment thereof or variant thereof of claim 1, wherein the light chain variable region comprises one or more of the amino acid sequences of SEQ ID NOs: 5, 7, 11, 15, 19, 39, 43, 61, 63, 65, 67, and 69.

4. The antibody or antigen-binding fragment thereof or variant thereof of claim 1, wherein the heavy chain variable region comprises one or more of the amino acid sequences of SEQ ID NOs: 21, 23, 27, 31, 35, 47, 51, 53, 55, 57, 59, and 68.

5. The antibody or antigen-binding fragment thereof or variant thereof of claim 1, wherein:
(a) the light chain variable region comprises an amino acid sequence with at least 75% identity to the amino acid sequence of SEQ ID NO: 5, or comprises the amino acid sequence of SEQ ID NO: 5; and/or the heavy chain variable region comprises an amino acid sequence with at least 75% identity to the amino acid sequence of SEQ ID NO: 21, or comprises the amino acid sequence of SEQ ID NO: 21;
(b) the light chain variable region comprises an amino acid sequence with at least 75% identity to the amino acid sequence of SEQ ID NO: 70, or comprises the amino acid sequence of SEQ ID NO: 70; and/or the heavy chain variable region comprises an amino acid sequence with at least 75% identity to the amino acid sequence of SEQ ID NO: 72, or comprises the amino acid sequence of SEQ ID NO: 72;
(c) the light chain variable region comprises an amino acid sequence with at least 75% identity to the amino acid sequence of SEQ ID NO: 71, or comprises the amino acid sequence of SEQ ID NO: 71; and/or the heavy chain variable region comprises an amino acid sequence with at least 75% identity to the amino acid sequence of SEQ ID NO: 73, or comprises the amino acid sequence of SEQ ID NO: 73; or
(d) the light chain variable region comprises an amino acid sequence with at least 75% identity to the amino acid sequence of SEQ ID NO: 5, or comprises the amino acid sequence of SEQ ID NO: 5; and/or the heavy chain variable region comprises an amino acid sequence with at least 75% identity to the amino acid sequence of SEQ ID NO: 74, or comprises the amino acid sequence of SEQ ID NO: 74.

6. The antibody or antigen-binding fragment thereof or variant thereof of claim 1, wherein the light chain variable region and the heavy chain variable region comprise a LCVR and HCVR amino acid sequence pair of SEQ ID NOs: 5 and 21, SEQ ID NOs: 70 and 72, SEQ ID NOs: 71 and 73, or SEQ ID NOs: 5 and 74.

7. The antibody or antigen-binding fragment thereof or variant thereof of claim 1, comprising a heavy chain/light chain sequence pair of SEQ ID NOs: 75 and 77, SEQ ID NOs: 76 and 78, SEQ ID NOs: 69 and 68, or SEQ ID NOs: 75 and 79.

8. The antibody or antigen-binding fragment thereof or variant thereof of claim 1, comprising at least one of the amino acid sequences of SEQ ID NOs: 3 and 37.

9. (canceled)

10. (canceled)

11. A bispecific or multiple-specific antibody or antigen-binding fragment thereof that binds two or more different epitopes on the same or different antigens, wherein one of the epitopes is on human prostate specific membrane antigen (PSMA), and the bispecific or multiple-specific antibody comprises the light chain variable region and/or the heavy chain variable region of the antibody or antigen-binding fragment thereof or variant thereof of claim 1.

12. An isolated chimeric antigen receptor (CAR), wherein the CAR comprises the antibody or antigen-binding fragment or variant of claim 1, a transmembrane domain, and an intracellular signaling domain comprising one or more stimulatory domains.

13. A nucleic acid molecule encoding a polypeptide chain of the antibody or antigen-binding fragment or variant of claim 1, a bispecific or multiple-specific antibody or antigen-binding fragment thereof comprising the light chain variable region and/or the heavy chain variable region of said antibody or antigen-binding fragment or variant thereof, or a CAR comprising the light chain variable region and/or the heavy chain variable region of said antibody or antigen-binding fragment or variant thereof.

14. A vector or a cell comprising the nucleic acid molecule of claim 13.

15. (canceled)

16. A method of preparing an antibody or antigen-binding fragment thereof, comprising:
obtaining the cell of claim 14;
culturing the cell in a medium under conditions permitting expression of a polypeptide encoded by the nucleic acid molecule and assembling of an antibody or fragment thereof; and
purifying the antibody or fragment from the cultured cell or the medium of the cell.

17. A composition comprising (i) the antibody or antigen-binding fragment or variant of claim 1, a bispecific or multiple-specific antibody or antigen-binding fragment thereof comprising the light chain variable region and/or the heavy chain variable region of said antibody or antigen-binding fragment or variant thereof, a CAR comprising the light chain variable region and/or the heavy chain variable region of said antibody or antigen-binding fragment or variant thereof, a nucleic acid molecule encoding a polypeptide chain of said antibody or antigen-binding fragment or variant or of said bispecific or multiple-specific antibody or antigen-binding fragment thereof, a vector comprising said nucleic acid molecule, or a cell comprising said nucleic acid molecule, and (ii) a pharmaceutically acceptable carrier.

18. (canceled)

19. (canceled)

20. A method of preventing or treating a proliferative disease associated with expression of PSMA in a subject, comprising administering to the subject an effective amount of the composition of claim 17.

21. The method of claim 20, further comprising administering to the subject a second agent or therapy.

22. (canceled)

23. A method of identifying a subject having a disease or condition associated with the expression of PSMA, comprising:
providing a tissue sample from the subject;
contacting the sample with the antibody or antigen-binding fragment thereof or variant thereof of claim 1; and
determining binding of the antibody or antigen-binding fragment thereof or variant thereof to the tissue sample, wherein the binding of the antibody or antigen-binding fragment thereof or variant thereof to the tissue sample is indicative of the presence of a disease condition associated with the expression of PSMA in the subject.

24. A method of imaging that targets PSMA, comprising administering to a subject in need thereof the antibody or antigen-binding fragment thereof or variant thereof of claim 1, wherein the antibody or antigen-binding fragment thereof or variant is linked to a reagent.

25. The method of claim 24, wherein the reagent comprises a photoactivatable agent, a fluorophore, a radioisotope, a bioluminescent protein, a bioluminescent peptide, a fluorescent tag, a fluorescent protein, a fluorescent peptide, an imaging contrast, an enzyme, a nuclear magnetic resonance active reagent, or a nanoparticle.

26. The method of claim 20, wherein the disease comprises a cancer.

27. (canceled)


SEQUENCE LISTING (82 sequences)

SEQ ID NO: 1 - protein, 707 aa, Homo sapiens
KSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 2 - protein, 756 aa, Homo sapiens
CAGTCTGTGCTGACTCAGCCGCCCTCAGTGTCTGGGGCCCCAGGGCAGAGTGTCATTATCTCCTGCACTGGGAGCAGCTCCAACATCGGGGCAGGTTCTCATGTACACTGGTACCAGCAGGTTCCAGGAACAGCCCCCAAACTCCTCATCTATGGAAACACCAATCGGCCCTCAGGGGTCCCTGACCGATTCTCTGGCTCCAAGTCTGGCACCTCAGGTTCCCTGGCCATCACTGGACTCCAGCCTGAGGATGAGGCTGATTATTATTGTGCAACATGGGATGACAGTCTGAGTGGTGTAATATTCGGCGGAGGGACCAAGGTCACCGTCCTAGGCGGATCCTCTAGGTCAAGTTCCAGCGGCGGCGGTGGCAGCGGAGGCGGCGGTGAGGTGCAGCTGGTGGAGTCTGGGGGAGCCCTGGCCAAGCCTGGGGGGTCCCTGAGACTCTCCTGTGCAGCCTCTGGATCCACCCTCAGTGGCTATGCTATGCACTGGGTCCGCCAGGCTCCAGGCAAGGGGCTGGAGTGGGTGGCAGTTATATCATATGATGGAAGCAATAAATACTACGCAGACTCCGTGAAGGGCCGATTCACCATCTCCAGAGACAATTCCAAGAACACGCTGTTTCTGCAAATGAACAGCCTGAGACCTGAGGACACGGCTGTGTACTATTGTGCTAAAGGCCTTACTTGGGGACTCGGTGACAATGATGCTCTCGATATCTGGGGCCCCGGGACCACGGTCACCGTCTCCTCA

SEQ ID NO: 3 - protein, 252 aa, Homo sapiens
QSVLTQPPSVSGAPGQSVIISCTGSSSNIGAGSHVHWYQQVPGTAPKLLIYGNTNRPSGVPDRFSGSKSGTSGSLAITGLQPEDEADYYCATWDDSLSGVIFGGGTKVTVLGGSSRSSSSGGGGSGGGGEVQLVESGGALAKPGGSLRLSCAASGSTLSGYAMHWVRQAPGKGLEWVAVISYDGSNKYYADSVKGRFTISRDNSKNTLFLQMNSLRPEDTAVYYCAKGLTWGLGDNDALDIWGPGTTVTVSS

SEQ ID NO: 4 - protein, 333 aa, Homo sapiens
CAGTCTGTGCTGACTCAGCCGCCCTCAGTGTCTGGGGCCCCAGGGCAGAGTGTCATTATCTCCTGCACTGGGAGCAGCTCCAACATCGGGGCAGGTTCTCATGTACACTGGTACCAGCAGGTTCCAGGAACAGCCCCCAAACTCCTCATCTATGGAAACACCAATCGGCCCTCAGGGGTCCCTGACCGATTCTCTGGCTCCAAGTCTGGCACCTCAGGTTCCCTGGCCATCACTGGACTCCAGCCTGAGGATGAGGCTGATTATTATTGTGCAACATGGGATGACAGTCTGAGTGGTGTAATATTCGGCGGAGGGACCAAGGTCACCGTCCTA

SEQ ID NO: 5 - protein, 111 aa, Homo sapiens
QSVLTQPPSVSGAPGQSVIISCTGSSSNIGAGSHVHWYQQVPGTAPKLLIYGNTNRPSGVPDRFSGSKSGTSGSLAITGLQPEDEADYYCATWDDSLSGVIFGGGTKVTVL

SEQ ID NO: 6 - DNA, 75 bp, Homo sapiens
  source 1..75: mol_type = unassigned DNA
cagtctgtgctgactcagccgccctcagtgtctggggccccagggcagagtgtcattatctcctgcactgggagc

SEQ ID NO: 7 - protein, 25 aa, Homo sapiens
QSVLTQPPSVSGAPGQSVIISCTGS

SEQ ID NO: 8 - DNA, 27 bp, Homo sapiens
  source 1..27: mol_type = unassigned DNA
agctccaacatcggggcaggttctcat

SEQ ID NO: 9 - protein, 9 aa, Homo sapiens
SSNIGAGSH

SEQ ID NO: 10 - DNA, 51 bp, Homo sapiens
  source 1..51: mol_type = unassigned DNA
gtacactggtaccagcaggttccaggaacagcccccaaactcctcatctat

SEQ ID NO: 11 - protein, 17 aa, Homo sapiens
VHWYQQVPGTAPKLLIY

SEQ ID NO: 12-13 - skipped (2 entries)

SEQ ID NO: 14 - DNA, 108 bp, Homo sapiens
  source 1..108: mol_type = unassigned DNA
aatcggccctcaggggtccctgaccgattctctggctccaagtctggcacctcaggttccctggccatcactggactccagcctgaggatgaggctgattattattgt

SEQ ID NO: 15 - protein, 36 aa, Homo sapiens
NRPSGVPDRFSGSKSGTSGSLAITGLQPEDEADYYC

SEQ ID NO: 16 - DNA, 33 bp, Homo sapiens
  source 1..33: mol_type = unassigned DNA
gcaacatgggatgacagtctgagtggtgtaata

SEQ ID NO: 17 - protein, 11 aa, Homo sapiens
ATWDDSLSGVI

SEQ ID NO: 18 - DNA, 30 bp, Homo sapiens
  source 1..30: mol_type = unassigned DNA
ttcggcggagggaccaaggtcaccgtccta

SEQ ID NO: 19 - protein, 10 aa, Homo sapiens
FGGGTKVTVL

SEQ ID NO: 20 - DNA, 369 bp, Homo sapiens
  source 1..369: mol_type = unassigned DNA
gaggtgcagctggtggagtctgggggagccctggccaagcctggggggtccctgagactctcctgtgcagcctctggatccaccctcagtggctatgctatgcactgggtccgccaggctccaggcaaggggctggagtgggtggcagttatatcatatgatggaagcaataaatactacgcagactccgtgaagggccgattcaccatctccagagacaattccaagaacacgctgtttctgcaaatgaacagcctgagacctgaggacacggctgtgtactattgtgctaaaggccttacttggggactcggtgacaatgatgctctcgatatctggggccccgggaccacggtcaccgtctcctca

SEQ ID NO: 21 - protein, 123 aa, Homo sapiens
EVQLVESGGALAKPGGSLRLSCAASGSTLSGYAMHWVRQAPGKGLEWVAVISYDGSNKYYADSVKGRFTISRDNSKNTLFLQMNSLRPEDTAVYYCAKGLTWGLGDNDALDIWGPGTTVTVSS

SEQ ID NO: 22 - DNA, 75 bp, Homo sapiens
  source 1..75: mol_type = unassigned DNA
gaggtgcagctggtggagtctgggggagccctggccaagcctggggggtccctgagactctcctgtgcagcctct

SEQ ID NO: 23 - protein, 25 aa, Homo sapiens
EVQLVESGGALAKPGGSLRLSCAAS

SEQ ID NO: 24 - DNA, 24 bp, Homo sapiens
  source 1..24: mol_type = unassigned DNA
ggatccaccctcagtggctatgct

SEQ ID NO: 25 - protein, 8 aa, Homo sapiens
GSTLSGYA

SEQ ID NO: 26 - DNA, 51 bp, Homo sapiens
  source 1..51: mol_type = unassigned DNA
atgcactgggtccgccaggctccaggcaaggggctggagtgggtggcagtt

SEQ ID NO: 27 - protein, 17 aa, Homo sapiens
MHWVRQAPGKGLEWVAV

SEQ ID NO: 28 - DNA, 24 bp, Homo sapiens
  source 1..24: mol_type = unassigned DNA
atatcatatgatggaagcaataaa

SEQ ID NO: 29 - protein, 8 aa, Homo sapiens
ISYDGSNK

SEQ ID NO: 30 - DNA, 114 bp, Homo sapiens
  source 1..114: mol_type = unassigned DNA
tactacgcagactccgtgaagggccgattcaccatctccagagacaattccaagaacacgctgtttctgcaaatgaacagcctgagacctgaggacacggctgtgtactattgt

SEQ ID NO: 31 - protein, 38 aa, Homo sapiens
YYADSVKGRFTISRDNSKNTLFLQMNSLRPEDTAVYYC

SEQ ID NO: 32 - DNA, 48 bp, Homo sapiens
  source 1..48: mol_type = unassigned DNA
gctaaaggccttacttggggactcggtgacaatgatgctctcgatatc

SEQ ID NO: 33 - protein, 16 aa, Homo sapiens
AKGLTWGLGDNDALDI

SEQ ID NO: 34 - DNA, 33 bp, Homo sapiens
  source 1..33: mol_type = unassigned DNA
tggggccccgggaccacggtcaccgtctcctca

SEQ ID NO: 35 - protein, 11 aa, Homo sapiens
WGPGTTVTVSS

SEQ ID NO: 36 - DNA, 54 bp, Homo sapiens
  source 1..54: mol_type = unassigned DNA
ggcggatcctctaggtcaagttccagcggcggcggtggcagcggaggcggcggt

SEQ ID NO: 37 - DNA, 18 bp, Homo sapiens
  source 1..18: mol_type = unassigned DNA
ggssrssssggggsgggg

SEQ ID NO: 38 - DNA, 51 bp, Homo sapiens
  source 1..51: mol_type = unassigned DNA
gtacactggtaccagcaggctccaggaacagcccccaaactcctcatctat

SEQ ID NO: 39 - protein, 17 aa, Homo sapiens
VHWYQQAPGTAPKLLIY

SEQ ID NO: 40-41 - skipped (2 entries)

SEQ ID NO: 42 - DNA, 30 bp, Homo sapiens
  source 1..30: mol_type = unassigned DNA
ttcggcggagggaccaaggccaccgtccta

SEQ ID NO: 43 - protein, 10 aa, Homo sapiens
FGGGTKATVL

SEQ ID NO: 44 - DNA, 24 bp, Homo sapiens
  source 1..24: mol_type = unassigned DNA
ggattcaccctcagtggctatgct

SEQ ID NO: 45 - protein, 8 aa, Homo sapiens
GFTLSGYA

SEQ ID NO: 46 - protein, 114 aa, Homo sapiens
TACTACGCAGACTCCGTGAAGGGCCGATTCACCGTCTCCAGAGACAATTCCAAGAACACGCTGTTTCTGCAAATGAACAGCCTGAGACCTGAGGACACGGCTGTGTACTATTGT

SEQ ID NO: 47 - protein, 38 aa, Homo sapiens
YYADSVKGRFTVSRDNSKNTLFLQMNSLRPEDTAVYYC

SEQ ID NO: 48 - DNA, 48 bp, Homo sapiens
  source 1..48: mol_type = unassigned DNA
gctaaaggccttacttggggactcggtgacaatgatgctctcggtatc

SEQ ID NO: 49 - protein, 16 aa, Homo sapiens
AKGLTWGLGDNDALGI

SEQ ID NO: 50 - DNA, 33 bp, Homo sapiens
  source 1..33: mol_type = unassigned DNA
tggggccccgagaccacggtcaccgtctcctca

SEQ ID NO: 51 - protein, 11 aa, Homo sapiens
WGPETTVTVSS

SEQ ID NO: 52 - DNA, 1419 bp, Homo sapiens
  source 1..1419: mol_type = unassigned DNA
atggagtttgggctgagctgggttttcctcgttgctcttttaagaggtgtccagtgtgaggtgcagctggtggagtctgggggagccctggccaagcctggggggtccctgagactctcctgtgcagcctctggattcaccctcagtggctatgctatgcactgggtccgccaggctccaggcaaggggctggagtgggtggcagttatatcatatgatggaagcaataaatactacgcagactccgtgaagggccgattcaccgtctccagagacaattccaagaacacgctgtttctgcaaatgaacagcctgagacctgaggacacggctgtgtactattgtgctaaaggccttacctggggactcggtgacaatgatgctctcgatatctggggccccgggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 53 - protein, 472 aa, Homo sapiens
MEFGLSWVFLVALLRGVQCEVQLVESGGALAKPGGSLRLSCAASGFTLSGYAMHWVRQAPGKGLEWVAVISYDGSNKYYADSVKGRFTVSRDNSKNTLFLQMNSLRPEDTAVYYCAKGLTWGLGDNDALDIWGPGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - DNA, 57 bp, Homo sapiens
  source 1..57: mol_type = unassigned DNA
atggagtttgggctgagctgggttttcctcgttgctcttttaagaggtgtccagtgt

SEQ ID NO: 55 - protein, 19 aa, Homo sapiens
MEFGLSWVFLVALLRGVQC

SEQ ID NO: 56 - DNA, 369 bp, Homo sapiens
  source 1..369: mol_type = unassigned DNA
gaggtgcagctggtggagtctgggggagccctggccaagcctggggggtccctgagactctcctgtgcagcctctggattcaccctcagtggctatgctatgcactgggtccgccaggctccaggcaaggggctggagtgggtggcagttatatcatatgatggaagcaataaatactacgcagactccgtgaagggccgattcaccgtctccagagacaattccaagaacacgctgtttctgcaaatgaacagcctgagacctgaggacacggctgtgtactattgtgctaaaggccttacctggggactcggtgacaatgatgctctcgatatctggggccccgggaccacggtcaccgtctcctca

SEQ ID NO: 57 - protein, 123 aa, Homo sapiens
EVQLVESGGALAKPGGSLRLSCAASGFTLSGYAMHWVRQAPGKGLEWVAVISYDGSNKYYADSVKGRFTVSRDNSKNTLFLQMNSLRPEDTAVYYCAKGLTWGLGDNDALDIWGPGTTVTVSS

SEQ ID NO: 58 - DNA, 990 bp, Homo sapiens
  source 1..990: mol_type = unassigned DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 59 - protein, 330 aa, Homo sapiens [sequence omitted]

SEQ ID NO: 60 - DNA, 711 bp, Homo sapiens
  source 1..711: mol_type = unassigned DNA
atggcctggtctcctctcctcctcactctcctcgctcactgcacagggtcctgggcccagtctgtgctgactcagccgccctcagtgtctggggccccagggcagagtgtcattatctcctgcactgggagcagctccaacatcggggcaggttctcatgtacactggtaccagcaggttccaggaacagcccccaaactcctcatctatgaaaacaccaatcggccctcaggggtccctgaccgattctctggctccaagtctggcacctcaggttccctggccatcactggactccagcctgaggatgaggctgattattattgtgcaacatgggatgacagtctgagtggtgtaatattcggcggagggaccaaggccaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 61 - protein, 236 aa, Homo sapiens
MAWSPLLLTLLAHCTGSWAQSVLTQPPSVSGAPGQSVIISCTGSSSNIGAGSHVHWYQQVPGTAPKLLIYENTNRPSGVPDRFSGSKSGTSGSLAITGLQPEDEADYYCATWDDSLSGVIFGGGTKATVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 62 - DNA, 57 bp, Homo sapiens
  source 1..57: mol_type = unassigned DNA
atggcctggtctcctctcctcctcactctcctcgctcactgcacagggtcctgggcc

SEQ ID NO: 63 - protein, 19 aa, Homo sapiens
MAWSPLLLTLLAHCTGSWA

SEQ ID NO: 64 - DNA, 333 bp, Homo sapiens
  source 1..333: mol_type = unassigned DNA
cagtctgtgctgactcagccgccctcagtgtctggggccccagggcagagtgtcattatctcctgcactgggagcagctccaacatcggggcaggttctcatgtacactggtaccagcaggttccaggaacagcccccaaactcctcatctatgaaaacaccaatcggccctcaggggtccctgaccgattctctggctccaagtctggcacctcaggttccctggccatcactggactccagcctgaggatgaggctgattattattgtgcaacatgggatgacagtctgagtggtgtaatattcggcggagggaccaaggccaccgtccta

SEQ ID NO: 65 - protein, 111 aa, Homo sapiens
QSVLTQPPSVSGAPGQSVIISCTGSSSNIGAGSHVHWYQQVPGTAPKLLIYENTNRPSGVPDRFSGSKSGTSGSLAITGLQPEDEADYYCATWDDSLSGVIFGGGTKATVL

SEQ ID NO: 66 - DNA, 318 bp, Homo sapiens
  source 1..318: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 67 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 68 - protein, 453 aa, Homo sapiens
EVQLVESGGALAKPGGSLRLSCAASGFTLSGYAMHWVRQAPGKGLEWVAVISYDGSNKYYADSVKGRFTVSRDNSKNTLFLQMNSLRPEDTAVYYCAKGLTWGLGDNDALDIWGPGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 217 aa, Homo sapiens
QSVLTQPPSVSGAPGQSVIISCTGSSSNIGAGSHVHWYQQVPGTAPKLLIYENTNRPSGVPDRFSGSKSGTSGSLAITGLQPEDEADYYCATWDDSLSGVIFGGGTKATVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 70 - protein, 111 aa, Homo sapiens
QSVLTQPPSVSGAPGQSVIISCTGSSSNIGAGSHVHWYQQAPGTAPKLLIYGNTNRPSGVPDRFSGSKSGTSGSLAITGLQPEDEADYYCATWDDSLSGVIFGGGTKVTVL

SEQ ID NO: 71 - protein, 111 aa, Homo sapiens; identical to SEQ ID NO: 65

SEQ ID NO: 72 - protein, 123 aa, Homo sapiens
EVQLVESGGALAKPGGSLRLSCAASGFTLSGYAMHWVRQAPGKGLEWVAVISYDGSNKYYADSVKGRFTISRDNSKNTLFLQMNSLRPEDTAVYYCAKGLTWGLGDNDALDIWGPETTVTVSS

SEQ ID NO: 73 - protein, 123 aa, Homo sapiens; identical to SEQ ID NO: 57

SEQ ID NO: 74 - protein, 123 aa, Homo sapiens
EVQLVESGGALAKPGGSLRLSCAASGFTLSGYAMHWVRQAPGKGLEWVAVISYDGSNKYYADSVKGRFTISRDNSKNTLFLQMNSLRPEDTAVYYCAKGLTWGLGDNDALGIWGPGTTVTVSS

SEQ ID NO: 75 - protein, 217 aa, Homo sapiens
QSVLTQPPSVSGAPGQSVIISCTGSSSNIGAGSHVHWYQQVPGTAPKLLIYGNTNRPSGVPDRFSGSKSGTSGSLAITGLQPEDEADYYCATWDDSLSGVIFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 76 - protein, 217 aa, Homo sapiens
QSVLTQPPSVSGAPGQSVIISCTGSSSNIGAGSHVHWYQQAPGTAPKLLIYGNTNRPSGVPDRFSGSKSGTSGSLAITGLQPEDEADYYCATWDDSLSGVIFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 77 - protein, 453 aa, Homo sapiens
EVQLVESGGALAKPGGSLRLSCAASGSTLSGYAMHWVRQAPGKGLEWVAVISYDGSNKYYADSVKGRFTISRDNSKNTLFLQMNSLRPEDTAVYYCAKGLTWGLGDNDALDIWGPGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 453 aa, Homo sapiens
EVQLVESGGALAKPGGSLRLSCAASGFTLSGYAMHWVRQAPGKGLEWVAVISYDGSNKYYADSVKGRFTISRDNSKNTLFLQMNSLRPEDTAVYYCAKGLTWGLGDNDALDIWGPETTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 453 aa, Homo sapiens
EVQLVESGGALAKPGGSLRLSCAASGFTLSGYAMHWVRQAPGKGLEWVAVISYDGSNKYYADSVKGRFTISRDNSKNTLFLQMNSLRPEDTAVYYCAKGLTWGLGDNDALGIWGPGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - DNA, 33 bp, Homo sapiens
  source 1..33: mol_type = unassigned DNA
gcaacatgggatgacagtctgaatggtgtaata

SEQ ID NO: 81 - DNA, 33 bp, Homo sapiens; identical to SEQ ID NO: 16
  source 1..33: mol_type = unassigned DNA

SEQ ID NO: 82 - protein, 11 aa, Homo sapiens
ATWDDSLNGVI